SEQUENCE LISTING (256 sequences)

SEQ ID NO: 1 - protein, 122 aa, synthetic construct
  REGION 1..122: note = Description of Artificial Sequence: Synthetic polypeptide
  REGION 1..122: note = BKO_1A1_VH
QLQLQESGPGLVKPSETLSLTCTVSGGSIRTSSYYWGWIRQPPGKGLEYIGSIYYSGTTYYNPSLKSRVTMSVDTSKNQFSLKMSSVTAADSAVYYCARHGRVREVPPFDYWGQGTLVTVSS

SEQ ID NO: 2 - protein, 108 aa, synthetic construct
  REGION 1..108: note = Description of Artificial Sequence: Synthetic polypeptide
  REGION 1..108: note = BKO_1A1_VL
SSELTQDPAVSVALGQTVRITCQGDSLRYYYASWYQQKPGQAPVLVIYDENSRPSGIPDRFSGSSSGNTASLSITGTQAEDEADYYCNSRDTSGNHWAFGGGTKLTVL

SEQ ID NO: 3 - protein, 122 aa, synthetic construct
  REGION 1..122: note = Description of Artificial Sequence: Synthetic polypeptide
  REGION 1..122: note = BKO_1B10_VH
QLQLQESGPGLVKPSETLSLTCTVSGGSIRTSSYYWGWIRQPPGKGLEYIGSIYYSGTTYYNPSLKSRVTMSVDTSKNQFSLKLSSVTAADTAVYYCARHGRVREVPPFDYWGQGTLVTVSS

SEQ ID NO: 4 - protein, 108 aa, synthetic construct
  REGION 1..108: note = Description of Artificial Sequence: Synthetic polypeptide
  REGION 1..108: note = BKO_1B10_VL
SSELTQDPAVSVALGQTVRITCQGDSLRYYYASWYQQKPGQAPVLVIYDENSRPSGIPDRFSGSSSGNTASLRITGTQAEDEADYYCNSRDTSGNHWAFGGGTKLTVL

SEQ ID NO: 5 - protein, 113 aa, synthetic construct
  REGION 1..113: note = Description of Artificial Sequence: Synthetic polypeptide
  REGION 1..113: note = BKO_1D1_VH
EVQLLESGGGLVQPGGSLRLSCAASKLTFKNSAMSWVRQAPGKGLEWVSAITGSGGRTYYADSVKGRFTISRDNSKNTLYLQMNSLRAEDTAIYYCAIQMGYWGQGILVTVSS

SEQ ID NO: 6 - protein, 111 aa, synthetic construct
  REGION 1..111: note = Description of Artificial Sequence: Synthetic polypeptide
  REGION 1..111: note = BKO_1D1_VL
QSALTQPPSASGSPGQSVTISCTGTSSDVGGYNYVSWYQQHPGKAPKLMIYEVNKRPSGVPARFSGSKSGNTASLTVSGLQAEDEADYYCSSYAGSNNFGVFGGGTKLTVL

SEQ ID NO: 7 - protein, 113 aa, synthetic construct
  REGION 1..113: note = Description of Artificial Sequence: Synthetic polypeptide
  REGION 1..113: note = BKO_1H3_VH
EVQLLESGGGLVQPGGSLRLSCAASGFTLSRSSTSWVRQTPGKGLEWVSAISGSGGRTYYADSVKGRFTISRDNSKNTLYLQMNSLRAEDTAVYYCAIQLGYWGQGILVTVSS

SEQ ID NO: 8 - protein, 110 aa, synthetic construct
  REGION 1..110: note = Description of Artificial Sequence: Synthetic polypeptide
  REGION 1..110: note = BKO_1H3_VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTWVFGGGTKLTVL

SEQ ID NO: 9 - protein, 113 aa, synthetic construct
  REGION 1..113: note = Description of Artificial Sequence: Synthetic polypeptide
  REGION 1..113: note = BKO_2D8_VH
EVQLLESGGGLVQPGGSLRLSCAASGYTFTSSTMSWVRQAPGKGLEWVTAISGRGGRTYYADSVKGRFTISRDNSKNTLYLQMNSLRAEDTAVYYCAIQLGNWGQGILVTVSS

SEQ ID NO: 10 - protein, 111 aa, synthetic construct
  REGION 1..111: note = Description of Artificial Sequence: Synthetic polypeptide
  REGION 1..111: note = BKO_2D8_VL
QSALTQPPSASGSPGQSVTISCTGTSSDIGGYNYVSWYQQHPGKAPKLVIYEVNMRPSGVPARFSGSKSGNTASLTVSGLQAEDEADYYCSSYAGNDNFGVFGGGTKLSVL

SEQ ID NO: 11 - protein, 123 aa, synthetic construct
  REGION 1..123: note = Description of Artificial Sequence: Synthetic polypeptide
  REGION 1..123: note = BKO_3A9_VH
QVQVQQSGPGLVKPSQTLSLTCAISGDSVSSNSAAWNWIRQSPSRGLEWLGRTYYRSKWYNDYAVSLKRRITIRPDTSRNHFSLHLSSVTPEDTAVYYCVRAYCGGGSCLDYWGQGTLVTVSS

SEQ ID NO: 12 - protein, 110 aa, synthetic construct
  REGION 1..110: note = Description of Artificial Sequence: Synthetic polypeptide
  REGION 1..110: note = BKO_3A9_L3_E03_VL
QSALTQPASASGSPGQSITISCTGTSSDVGNYNRVSWYQQHPGKVPKLMIYEGSKRPSGISNRFSGSKSGNTASLTISGLQPEDEADYFCCSYAGSNTLVFGGGTKLTVL

SEQ ID NO: 13 - protein, 123 aa, synthetic construct
  REGION 1..123: note = Description of Artificial Sequence: Synthetic polypeptide
  REGION 1..123: note = BKO_3D6_VH
EVQLVESGGDLVQPGRSLRLSCAASGFTFDDYAMHWVRQAPGKGLKWVSGITWNSGNKRYADSVKGRFTISRDNAKNSLYLQMNSLRAEDTALYYCAKDMKGSGTYFPAFDYWGQGTLVTVSS

SEQ ID NO: 14 - protein, 106 aa, synthetic construct
  REGION 1..106: note = Description of Artificial Sequence: Synthetic polypeptide
  REGION 1..106: note = BKO_3D6_L6_G06_VL (BKO_5H4_VL)
SYELTQPPSVSVSPGQTASITCSGDKLGDKYACWYQQKPGQSPVLVIYQDSKRPSGIPERFSGSNSGNTATLTISGTQAMDEADYYCQAWDSSTVVFGGGTKLTVL

SEQ ID NO: 15 - protein, 113 aa, synthetic construct
  REGION 1..113: note = Description of Artificial Sequence: Synthetic polypeptide
  REGION 1..113: note = BKO_3F4_VH
EVQLLESGGGLVQPGGSLRLSCAASGLTFSSYAMSWVRQAPGKGLEWVSAISGSGGKIYYADSVKGRFTISRDNSKNTLYLQMNSLRAEDTAVYYCAIQVGYWGQGTLVTVSS

SEQ ID NO: 16 - protein, 111 aa, synthetic construct
  REGION 1..111: note = Description of Artificial Sequence: Synthetic polypeptide
  REGION 1..111: note = BKO_3F4_L11_A11_VL
QSALTQPPSASGSPGQSVTMSCTGTSSDVGGYNYVTWYQQHPGKAPKLMIYEVSKRPSGVPARFSGSKSGNTASLTVSGLQAEDEADYYCSSYAGPNNFGVFGGGTKLTVL

SEQ ID NO: 17 - protein, 113 aa, synthetic construct
  REGION 1..113: note = Description of Artificial Sequence: Synthetic polypeptide
  REGION 1..113: note = BKO_4A8_VH
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSS

SEQ ID NO: 18 - protein, 111 aa, synthetic construct
  REGION 1..111: note = Description of Artificial Sequence: Synthetic polypeptide
  REGION 1..111: note = BKO_4A8_VL
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNNFGVFGGGTKLTVL

SEQ ID NO: 19 - protein, 123 aa, synthetic construct
  REGION 1..123: note = Description of Artificial Sequence: Synthetic polypeptide
  REGION 1..123: note = BKO_4F10_VH
QVQLVQSGAEVKKPGASVKVSCKASGYTFTGYYIHWVRQAPGQGLEWMGRFNPNNGGTNYAQRFQGRVTMTRDTSISTAYMELSRLRSDDTAVYYCARGPTIRLWFDNWFDSWGQGTLVTVSS

SEQ ID NO: 20 - protein, 110 aa, synthetic construct
  REGION 1..110: note = Description of Artificial Sequence: Synthetic polypeptide
  REGION 1..110: note = BKO_4F10_VL
QSALTQPASVSGSPGQSITISCTGTSSDVGGYNYVSWYQQHPGKAPKLMIYDVSNRPSGISNRFSGSKSGNTASLTISGLQAEDEADYYCSSYTSSSTWVFGGGTKLTVL

SEQ ID NO: 21 - protein, 122 aa, synthetic construct
  REGION 1..122: note = Description of Artificial Sequence: Synthetic polypeptide
  REGION 1..122: note = BKO_5E8_H5_C05_VH
EVQLLESGGGLVQPGGSLRLSCAASGFTFSSYAMSWVRQAPGRGLEWVSAIRGSGAGTYYADSMKGRFTISRDNSKDTLYLLMNSLRAEDTAVYYCSKLEAVSGTGKYFQHWGQGTLVTVSS

SEQ ID NO: 22 - protein, 106 aa, synthetic construct
  REGION 1..106: note = Description of Artificial Sequence: Synthetic polypeptide
  REGION 1..106: note = BKO_5E8_L3_C03_VL
SYELTQPPSVSVSPGQTANITCSGDTLGDKFACWYQQKPGQSPVLVIYQDTKRPSGIPERFSGSKSGITATLTISGTQAMDEADFYCQAWNSRGVVFGGGTRLTVL

SEQ ID NO: 23 - protein, 112 aa, synthetic construct
  REGION 1..112: note = Description of Artificial Sequence: Synthetic polypeptide
  REGION 1..112: note = BKO_5G11_VH
EVQLLESGGGLVQPGGSLRLSCAVSGFTFSNYAMTWVRQAPGKGLEWVSAISGRGSRTYYADSVKGRFTISRDTSKNTLYLQMNSLRAEDTAVYYCAKMDYWGQGTLVTVSS

SEQ ID NO: 24 - protein, 111 aa, synthetic construct
  REGION 1..111: note = Description of Artificial Sequence: Synthetic polypeptide
  REGION 1..111: note = BKO_5G11_VL
QSALTQPPSASGSPGQSVTISCTGTSSDVGGYNYVSWYQQHPGKAPKLMIFEVSKRPSGVPDRFSGSKSGNTASLTVSGLQAEDEADYYCSSYAGSNNFGVFGGGTKLTVL

SEQ ID NO: 25 - protein, 123 aa, synthetic construct
  REGION 1..123: note = Description of Artificial Sequence: Synthetic polypeptide
  REGION 1..123: note = BKO_5G6_VH
QVQLVQSGAEVTKPGASVKVSCKASGYTFTGYYIHWVRQAPGQGLEWMGRFNPNNGGTNYAQRFQGRVTMTRDTSISTAYMELSRLRSDDTAVYYCARGPTIRLWFDNWFDSWGQGTLVTVSS

SEQ ID NO: 26 - protein, 110 aa, synthetic construct
  REGION 1..110: note = Description of Artificial Sequence: Synthetic polypeptide
  REGION 1..110: note = BKO_5G6_L12_E12_VL
QSALTQPASVSGSPGQSITISCTGTSSDVGGYNYVSWYQQHPGKAPKLMIYDVSNRPSGISNRFSGSKSGNTASLTISGLQAEDEADYYCSSYTSSSTWVFGGGTTLTVL

SEQ ID NO: 27 - protein, 123 aa, synthetic construct
  REGION 1..123: note = Description of Artificial Sequence: Synthetic polypeptide
  REGION 1..123: note = BKO_6A1_H4_A04_VH
QVQLKQWGAGLLKPSETLSLTCAVYGGSFSGYYWTWIRQPPGKGLEWIGEINHSGSTNYNPSLKSRVTMSVDTSKNQFSLKLRSVTAADTAVYYCARGEVRGLITLYWYFDVWGRGSLVTVSS

SEQ ID NO: 28 - protein, 108 aa, synthetic construct
  REGION 1..108: note = Description of Artificial Sequence: Synthetic polypeptide
  REGION 1..108: note = BKO_6A1_E10_VL
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 29 - protein, 120 aa, synthetic construct
  REGION 1..120: note = Description of Artificial Sequence: Synthetic polypeptide
  REGION 1..120: note = BKO_6A2_H1_B01_VH
QVQLVESGGGVVQPGRSLRLSCAASGFTFSTYDIHWVRQAPGKGLEWVAVIWYDGSNKYYADSVKGRFTISRDNSKNTLYLQMNSLRAEDTAVYYCARDEGYNYGYGGYWGQGTLVTVSS

SEQ ID NO: 30 - protein, 106 aa, synthetic construct; identical to SEQ ID NO: 14
  REGION 1..106: note = Description of Artificial Sequence: Synthetic polypeptide
  REGION 1..106: note = BKO_6A2_L6_A06_VL

SEQ ID NO: 31 - protein, 123 aa, synthetic construct
  REGION 1..123: note = Description of Artificial Sequence: Synthetic polypeptide
  REGION 1..123: note = BKO_7C11_H6_B06_VH
QVQLQQWGAGLLKPSETLSLTCAVYGGSFSGYYWSWIRQPPGKGLEWIGEINHSRNTNYNPSLKSRVTISVDTSKNQFSLKLSSVTAADTAVYYCARGEVRGVFTLYWYFDVWGRGTLVTVSS

SEQ ID NO: 32 - protein, 108 aa, synthetic construct
  REGION 1..108: note = Description of Artificial Sequence: Synthetic polypeptide
  REGION 1..108: note = BKO_7C11_G01_VL
SSELTQGPAVSVALGQTVRITCQGNSLRFYYASWYQQRPGQAPILVIYDKNNRPSGIPDRFSGSSSGNTASLTITGAQAEDEADYYCNSRDSSGYYMIFGGGTKLTVL

SEQ ID NO: 33 - protein, 112 aa, synthetic construct; identical to SEQ ID NO: 23
  REGION 1..112: note = Description of Artificial Sequence: Synthetic polypeptide
  REGION 1..112: note = BKO_7G10_H1_B01_VH

SEQ ID NO: 34 - protein, 111 aa, synthetic construct; identical to SEQ ID NO: 24
  REGION 1..111: note = Description of Artificial Sequence: Synthetic polypeptide
  REGION 1..111: note = BKO_7G10_L6_E06_VL

SEQ ID NO: 35 - protein, 113 aa, synthetic construct; identical to SEQ ID NO: 9
  REGION 1..113: note = Description of Artificial Sequence: Synthetic polypeptide
  REGION 1..113: note = BKO_7H8_H3_C03_VH

SEQ ID NO: 36 - protein, 111 aa, synthetic construct; identical to SEQ ID NO: 10
  REGION 1..111: note = Description of Artificial Sequence: Synthetic polypeptide
  REGION 1..111: note = BKO_7H8_L10_F10_VL

SEQ ID NO: 37 - protein, 113 aa, synthetic construct
  REGION 1..113: note = Description of Artificial Sequence: Synthetic polypeptide
  REGION 1..113: note = BKO_8B6_VH
EVQLLESGGGLVQPGGSLRLSCAASGFTFSSNAMSWVRQAPGKGLEWVSAISNSGRSTYYADSVKGRFTISRDSSKNTLYLLMNSLRAEDSAVYYCAIKLGYWGQGSLVTVSS

SEQ ID NO: 38 - protein, 111 aa, synthetic construct
  REGION 1..111: note = Description of Artificial Sequence: Synthetic polypeptide
  REGION 1..111: note = BKO_8B6_VL
QSALTQPPSASGSPGQSVTISCTGTSSDVGGYNYVSWYQQHPGKAPKLMMYEVSKRPSGVPDRFSGSKSGNTASLTVSGLQAEDEADYYCSSYAGSDNFGVFGGGTRLTVL

SEQ ID NO: 39 - protein, 122 aa, synthetic construct; identical to SEQ ID NO: 3
  REGION 1..122: note = Description of Artificial Sequence: Synthetic polypeptide
  REGION 1..122: note = BKO_8C4_VH

SEQ ID NO: 40 - protein, 108 aa, synthetic construct; identical to SEQ ID NO: 4
  REGION 1..108: note = Description of Artificial Sequence: Synthetic polypeptide
  REGION 1..108: note = BKO_8C4_VL

SEQ ID NO: 41 - protein, 113 aa, synthetic construct
  REGION 1..113: note = Description of Artificial Sequence: Synthetic polypeptide
  REGION 1..113: note = BKO_8G3_H4_D04_VH
EVQLLESGGGLVQPGGSLRLSCAASGFTFSSYAMSWVRQAPGKGLEWVSAITGSGGSTYYADSVKGRFTISRDKSKNTLYLQMNSLRAEDTAVYYCAIRLGYWGQGSLVTVSS

SEQ ID NO: 42 - protein, 111 aa, synthetic construct
  REGION 1..111: note = Description of Artificial Sequence: Synthetic polypeptide
  REGION 1..111: note = BKO_8G3_L1_G01_VL
QSALTQPPSASGSPGQSVTISCTGTSSDVGGYNYVSWYQQHPGKVPKLVIYEVSKRPSGVPDRFSGSKSGNTASLTVSGLQAEDEADYYCSSYAGSNNFGVFGGGTKLTVL

SEQ ID NO: 43 - protein, 116 aa, synthetic construct
  REGION 1..116: note = Description of Artificial Sequence: Synthetic polypeptide
  REGION 1..116: note = BKO_8H10_VH
QVQLVQSGAEVKKPGASVKVSCKASGYTFTGYYIHWVRQAPGQGLEWMGRIKPDSGGTNYAQKFQGRVTMTRDTSITTAYMELSRLRSDDTAVYYCARGGSGWDYWGQGTLVTVSS

SEQ ID NO: 44 - protein, 110 aa, synthetic construct
  REGION 1..110: note = Description of Artificial Sequence: Synthetic polypeptide
  REGION 1..110: note = BKO_8H10_VL
QSALTQPPSASGSPGQSVTISFTGTSRDVGDYNYVSWYQQHPGKAPKLMIYEVNKRPSGVPDRFSGSKSGNTASLTVSGLQAEDEADYYCSSYAGSNTYVFGTGTKVTVL

SEQ ID NO: 45 - protein, 113 aa, synthetic construct
  REGION 1..113: note = Description of Artificial Sequence: Synthetic polypeptide
  REGION 1..113: note = BKO_8H8_H5_E05_VH
EVQLLESGGGLVQPGGSLRLSCAASGLTVSSYAMSWVRQAPGKGLEWVSAISGSGGKIYYADSVKGRFTISRDNSKNTLYLQMNSLSAEDTAVYYCAIQVGYWGQGTLVTVSS

SEQ ID NO: 46 - protein, 111 aa, synthetic construct
  REGION 1..111: note = Description of Artificial Sequence: Synthetic polypeptide
  REGION 1..111: note = BKO_8H8_L7_H08_VL
QSALTQPPSASGSPGQSVTMSCTGTSSDVGGYNYVTWYQQHPGKAPKLVIYEVSKRPSGVPVRFSGSKSGNTASLTVSGLQAEDEADYYCSSYAGPNNFGIFGGGTKLTVL

SEQ ID NO: 47 - protein, 113 aa, synthetic construct
  REGION 1..113: note = Description of Artificial Sequence: Synthetic polypeptide
  REGION 1..113: note = BKO_9A8_H3_F03_VH
EVQLLESGGGLVQTGGSLRLSCAASGFTFSSNTMSWVRQAPGKGLEWVSAISGSGGRTYYVDSVKGRFTISRDNSKNTLYLQMHSLRAEDTAVYYCAIQLGSWGQGILVTVSS

SEQ ID NO: 48 - protein, 111 aa, synthetic construct
  REGION 1..111: note = Description of Artificial Sequence: Synthetic polypeptide
  REGION 1..111: note = BKO_9A8_L1_H02_VL
QSALTQPPSASGSPGQSVTISCTGTSSDVGAYNYVSWYQQHPGKAPKLMIYEVTKRPSGVPDRFSGSKSGNTASLTVSGLQAEDEADYYCSSYAGNNNFGVFGGGTKLTVL

SEQ ID NO: 49 - protein, 113 aa, synthetic construct
  REGION 1..113: note = Description of Artificial Sequence: Synthetic polypeptide
  REGION 1..113: note = BKO_9C3_H8_G08_VH
EVQVLESGGGLVQPGGSLRLSCAASGFTFRSYAMSWVRQAPGKGLEWVSAISGSGGSTYYADSVKGRFTISRDKSKNTLYLQMNSLRAEDTAVYYCAIQLGYWGQGTLVTVSS

SEQ ID NO: 50 - protein, 111 aa, synthetic construct
  REGION 1..111: note = Description of Artificial Sequence: Synthetic polypeptide
  REGION 1..111: note = BKO_9C3_L1_F01_VL
QSALTQPPSASGSPGQSVIISCTGTSSDVGGYNYVSWYQQHPGKVPKLMIYEVSKRPSGVPDRFSGSKSGNTASLTVSGLQAEDEADYYCTSYAGSNNFGVFGGGTKLTVL

SEQ ID NO: 51 - protein, 119 aa, synthetic construct
  REGION 1..119: note = Description of Artificial Sequence: Synthetic polypeptide
  REGION 1..119: note = BKO_10G10_VH
QLQLQESGPGLVKPSETLSLTCTVSGGSIRRSSYYWGWIRQPPGKGLEWIGSFYNSGNTYYKPSLKSRVAISVDTPKNQFSLKLSSVTAADTAVYYCARGYSSGGFDPWGQGTLVTVSS

SEQ ID NO: 52 - protein, 110 aa, synthetic construct
  REGION 1..110: note = Description of Artificial Sequence: Synthetic polypeptide
  REGION 1..110: note = BKO_10G10_VL
QSALTQPASVSGSPGQSITISCTGTSSDVGGYNYVSWCQQHPGKAPKIMIFDVSNRPSGVSNRFSGSKSGNTASLTISGLQAEDEADYYCSSYTSSSTWVFGGGTRLTVL

SEQ ID NO: 53 - protein, 113 aa, synthetic construct
  REGION 1..113: note = Description of Artificial Sequence: Synthetic polypeptide
  REGION 1..113: note = 4A8 VH S32Q
EVQLLESGGGLVQPGGSLRLSCAASGFTFSSQTMSWVRQAPGKGLEWVSAISGRGRNTYYADSVKGRFTISRDNSRNTLYLQMNSLRAEDTAVYYCAIQMGYWGQGILVTVSS

SEQ ID NO: 54 - protein, 113 aa, synthetic construct
  REGION 1..113: note = Description of Artificial Sequence: Synthetic polypeptide
  REGION 1..113: note = 4A8 VH S32H
EVQLLESGGGLVQPGGSLRLSCAASGFTFSSHTMSWVRQAPGKGLEWVSAISGRGRNTYYADSVKGRFTISRDNSRNTLYLQMNSLRAEDTAVYYCAIQMGYWGQGILVTVSS

SEQ ID NO: 55 - protein, 113 aa, synthetic construct
  REGION 1..113: note = Description of Artificial Sequence: Synthetic polypeptide
  REGION 1..113: note = 4A8 VH S32L
EVQLLESGGGLVQPGGSLRLSCAASGFTFSSLTMSWVRQAPGKGLEWVSAISGRGRNTYYADSVKGRFTISRDNSRNTLYLQMNSLRAEDTAVYYCAIQMGYWGQGILVTVSS

SEQ ID NO: 56 - protein, 113 aa, synthetic construct
  REGION 1..113: note = Description of Artificial Sequence: Synthetic polypeptide
  REGION 1..113: note = 4A8 VH S32W
EVQLLESGGGLVQPGGSLRLSCAASGFTFSSWTMSWVRQAPGKGLEWVSAISGRGRNTYYADSVKGRFTISRDNSRNTLYLQMNSLRAEDTAVYYCAIQMGYWGQGILVTVSS

SEQ ID NO: 57 - protein, 113 aa, synthetic construct
  REGION 1..113: note = Description of Artificial Sequence: Synthetic polypeptide
  REGION 1..113: note = 4A8 VH S32Y
EVQLLESGGGLVQPGGSLRLSCAASGFTFSSYTMSWVRQAPGKGLEWVSAISGRGRNTYYADSVKGRFTISRDNSRNTLYLQMNSLRAEDTAVYYCAIQMGYWGQGILVTVSS

SEQ ID NO: 58 - protein, 113 aa, synthetic construct
  REGION 1..113: note = Description of Artificial Sequence: Synthetic polypeptide
  REGION 1..113: note = 4A8 VH T33A
EVQLLESGGGLVQPGGSLRLSCAASGFTFSSSAMSWVRQAPGKGLEWVSAISGRGRNTYYADSVKGRFTISRDNSRNTLYLQMNSLRAEDTAVYYCAIQMGYWGQGILVTVSS

SEQ ID NO: 59 - protein, 113 aa, synthetic construct
  REGION 1..113: note = Description of Artificial Sequence: Synthetic polypeptide
  REGION 1..113: note = 4A8 VH M34Q
EVQLLESGGGLVQPGGSLRLSCAASGFTFSSSTQSWVRQAPGKGLEWVSAISGRGRNTYYADSVKGRFTISRDNSRNTLYLQMNSLRAEDTAVYYCAIQMGYWGQGILVTVSS

SEQ ID NO: 60 - protein, 113 aa, synthetic construct
  REGION 1..113: note = Description of Artificial Sequence: Synthetic polypeptide
  REGION 1..113: note = 4A8 VH M34D
EVQLLESGGGLVQPGGSLRLSCAASGFTFSSSTDSWVRQAPGKGLEWVSAISGRGRNTYYADSVKGRFTISRDNSRNTLYLQMNSLRAEDTAVYYCAIQMGYWGQGILVTVSS

SEQ ID NO: 61 - protein, 113 aa, synthetic construct
  REGION 1..113: note = Description of Artificial Sequence: Synthetic polypeptide
  REGION 1..113: note = 4A8 VH M34H
EVQLLESGGGLVQPGGSLRLSCAASGFTFSSSTHSWVRQAPGKGLEWVSAISGRGRNTYYADSVKGRFTISRDNSRNTLYLQMNSLRAEDTAVYYCAIQMGYWGQGILVTVSS

SEQ ID NO: 62 - protein, 113 aa, synthetic construct
  REGION 1..113: note = Description of Artificial Sequence: Synthetic polypeptide
  REGION 1..113: note = 4A8 VH M34W
EVQLLESGGGLVQPGGSLRLSCAASGFTFSSSTWSWVRQAPGKGLEWVSAISGRGRNTYYADSVKGRFTISRDNSRNTLYLQMNSLRAEDTAVYYCAIQMGYWGQGILVTVSS

SEQ ID NO: 63 - protein, 113 aa, synthetic construct
  REGION 1..113: note = Description of Artificial Sequence: Synthetic polypeptide
  REGION 1..113: note = 4A8 VH I51H
EVQLLESGGGLVQPGGSLRLSCAASGFTFSSSTMSWVRQAPGKGLEWVSAHSGRGRNTYYADSVKGRFTISRDNSRNTLYLQMNSLRAEDTAVYYCAIQMGYWGQGILVTVSS

SEQ ID NO: 64 - protein, 113 aa, synthetic construct
  REGION 1..113: note = Description of Artificial Sequence: Synthetic polypeptide
  REGION 1..113: note = 4A8 VH G52aD
EVQLLESGGGLVQPGGSLRLSCAASGFTFSSSTMSWVRQAPGKGLEWVSAISDRGRNTYYADSVKGRFTISRDNSRNTLYLQMNSLRAEDTAVYYCAIQMGYWGQGILVTVSS

SEQ ID NO: 65 - protein, 113 aa, synthetic construct
  REGION 1..113: note = Description of Artificial Sequence: Synthetic polypeptide
  REGION 1..113: note = 4A8 VH R53S
EVQLLESGGGLVQPGGSLRLSCAASGFTFSSSTMSWVRQAPGKGLEWVSAISGSGRNTYYADSVKGRFTISRDNSRNTLYLQMNSLRAEDTAVYYCAIQMGYWGQGILVTVSS

SEQ ID NO: 66 - protein, 113 aa, synthetic construct
  REGION 1..113: note = Description of Artificial Sequence: Synthetic polypeptide
  REGION 1..113: note = 4A8 VH R53Q
EVQLLESGGGLVQPGGSLRLSCAASGFTFSSSTMSWVRQAPGKGLEWVSAISGQGRNTYYADSVKGRFTISRDNSRNTLYLQMNSLRAEDTAVYYCAIQMGYWGQGILVTVSS

SEQ ID NO: 67 - protein, 113 aa, synthetic construct
  REGION 1..113: note = Description of Artificial Sequence: Synthetic polypeptide
  REGION 1..113: note = 4A8 VH G54D
EVQLLESGGGLVQPGGSLRLSCAASGFTFSSSTMSWVRQAPGKGLEWVSAISGRDRNTYYADSVKGRFTISRDNSRNTLYLQMNSLRAEDTAVYYCAIQMGYWGQGILVTVSS

SEQ ID NO: 68 - protein, 113 aa, synthetic construct
  REGION 1..113: note = Description of Artificial Sequence: Synthetic polypeptide
  REGION 1..113: note = 4A8 VH N56S
EVQLLESGGGLVQPGGSLRLSCAASGFTFSSSTMSWVRQAPGKGLEWVSAISGRGRSTYYADSVKGRFTISRDNSRNTLYLQMNSLRAEDTAVYYCAIQMGYWGQGILVTVSS

SEQ ID NO: 69 - protein, 113 aa, synthetic construct
  REGION 1..113: note = Description of Artificial Sequence: Synthetic polypeptide
  REGION 1..113: note = 4A8 VH I94K
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAKQMGYWGQGILVTVSS

SEQ ID NO: 70 - protein, 113 aa, synthetic construct
  REGION 1..113: note = Description of Artificial Sequence: Synthetic polypeptide
  REGION 1..113: note = 4A8 VH M96A
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQAGYWGQGILVTVSS

SEQ ID NO: 71 - protein, 113 aa, synthetic construct
  REGION 1..113: note = Description of Artificial Sequence: Synthetic polypeptide
  REGION 1..113: note = 4A8 VH M96Q
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQQGYWGQGILVTVSS

SEQ ID NO: 72 - protein, 113 aa, synthetic construct
  REGION 1..113: note = Description of Artificial Sequence: Synthetic polypeptide
  REGION 1..113: note = 4A8 VH M96K
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQKGYWGQGILVTVSS

SEQ ID NO: 73 - protein, 113 aa, synthetic construct
  REGION 1..113: note = Description of Artificial Sequence: Synthetic polypeptide
  REGION 1..113: note = 4A8 VH G101D
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMDYWGQGILVTVSS

SEQ ID NO: 74 - protein, 113 aa, synthetic construct
  REGION 1..113: note = Description of Artificial Sequence: Synthetic polypeptide
  REGION 1..113: note = 4A8 VH Y102S
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGSWGQGILVTVSS

SEQ ID NO: 75 - protein, 113 aa, synthetic construct
  REGION 1..113: note = Description of Artificial Sequence: Synthetic polypeptide
  REGION 1..113: note = 4A8 VH Y102K
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGKWGQGILVTVSS

SEQ ID NO: 76 - protein, 111 aa, synthetic construct
  REGION 1..111: note = Description of Artificial Sequence: Synthetic polypeptide
  REGION 1..111: note = 4A8 VL E50D
QSALTQPPSASGSPGQSVTISCIGTSSDVGGYNYVSWYQQHPDKAPKLMIYDVNKRPSGVPDRFSGSKSGNTASLTVSGLQAEDEADYYCSSYAGNNNFGVFGGGTKLTVL

SEQ ID NO: 77 - protein, 111 aa, synthetic construct
  REGION 1..111: note = Description of Artificial Sequence: Synthetic polypeptide
  REGION 1..111: note = 4A8 VL N52D
QSALTQPPSASGSPGQSVTISCIGTSSDVGGYNYVSWYQQHPDKAPKLMIYEVDKRPSGVPDRFSGSKSGNTASLTVSGLQAEDEADYYCSSYAGNNNFGVFGGGTKLTVL

SEQ ID NO: 78 - protein, 111 aa, synthetic construct
  REGION 1..111: note = Description of Artificial Sequence: Synthetic polypeptide
  REGION 1..111: note = 4A8 VL N52S
QSALTQPPSASGSPGQSVTISCIGTSSDVGGYNYVSWYQQHPDKAPKLMIYEVSKRPSGVPDRFSGSKSGNTASLTVSGLQAEDEADYYCSSYAGNNNFGVFGGGTKLTVL

SEQ ID NO: 79 - protein, 111 aa, synthetic construct
  REGION 1..111: note = Description of Artificial Sequence: Synthetic polypeptide
  REGION 1..111: note = 4A8 VL K53A
QSALTQPPSASGSPGQSVTISCIGTSSDVGGYNYVSWYQQHPDKAPKLMIYEVNARPSGVPDRFSGSKSGNTASLTVSGLQAEDEADYYCSSYAGNNNFGVFGGGTKLTVL

SEQ ID NO: 80 - protein, 111 aa, synthetic construct
  REGION 1..111: note = Description of Artificial Sequence: Synthetic polypeptide
  REGION 1..111: note = 4A8 VL K53D
QSALTQPPSASGSPGQSVTISCIGTSSDVGGYNYVSWYQQHPDKAPKLMIYEVNDRPSGVPDRFSGSKSGNTASLTVSGLQAEDEADYYCSSYAGNNNFGVFGGGTKLTVL

SEQ ID NO: 81 - protein, 111 aa, synthetic construct
  REGION 1..111: note = Description of Artificial Sequence: Synthetic polypeptide
  REGION 1..111: note = 4A8 VL K53H
QSALTQPPSASGSPGQSVTISCIGTSSDVGGYNYVSWYQQHPDKAPKLMIYEVNHRPSGVPDRFSGSKSGNTASLTVSGLQAEDEADYYCSSYAGNNNFGVFGGGTKLTVL

SEQ ID NO: 82 - protein, 111 aa, synthetic construct
  REGION 1..111: note = Description of Artificial Sequence: Synthetic polypeptide
  REGION 1..111: note = 4A8 VL R54Q
QSALTQPPSASGSPGQSVTISCIGTSSDVGGYNYVSWYQQHPDKAPKLMIYEVNKQPSGVPDRFSGSKSGNTASLTVSGLQAEDEADYYCSSYAGNNNFGVFGGGTKLTVL

SEQ ID NO: 83 - protein, 111 aa, synthetic construct
  REGION 1..111: note = Description of Artificial Sequence: Synthetic polypeptide
  REGION 1..111: note = 4A8 VL Y91A
QSALTQPPSASGSPGQSVTISCIGTSSDVGGYNYVSWYQQHPDKAPKLMIYEVNKRPSGVPDRFSGSKSGNTASLTVSGLQAEDEADYYCSSAAGNNNFGVFGGGTKLTVL

SEQ ID NO: 84 - protein, 111 aa, synthetic construct
  REGION 1..111: note = Description of Artificial Sequence: Synthetic polypeptide
  REGION 1..111: note = 4A8 VL N94A
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGANNFGVFGGGTKLTVL

SEQ ID NO: 85 - protein, 111 aa, synthetic construct
  REGION 1..111: note = Description of Artificial Sequence: Synthetic polypeptide
  REGION 1..111: note = 4A8 VL N94S
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGSNNFGVFGGGTKLTVL

SEQ ID NO: 86 - protein, 111 aa, synthetic construct
  REGION 1..111: note = Description of Artificial Sequence: Synthetic polypeptide
  REGION 1..111: note = 4A8 VL N94K
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGKNNFGVFGGGTKLTVL

SEQ ID NO: 87 - protein, 111 aa, synthetic construct
  REGION 1..111: note = Description of Artificial Sequence: Synthetic polypeptide
  REGION 1..111: note = 4A8 VL N94L
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGLNNFGVFGGGTKLTVL

SEQ ID NO: 88 - protein, 111 aa, synthetic construct
  REGION 1..111: note = Description of Artificial Sequence: Synthetic polypeptide
  REGION 1..111: note = 4A8 VL N94W
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGWNNFGVFGGGTKLTVL

SEQ ID NO: 89 - protein, 111 aa, synthetic construct
  REGION 1..111: note = Description of Artificial Sequence: Synthetic polypeptide
  REGION 1..111: note = 4A8 VL N94Y
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGYNNFGVFGGGTKLTVL

SEQ ID NO: 90 - protein, 111 aa, synthetic construct
  REGION 1..111: note = Description of Artificial Sequence: Synthetic polypeptide
  REGION 1..111: note = 4A8 VL N95aQ
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNQFGVFGGGTKLTVL

SEQ ID NO: 91 - protein, 111 aa, synthetic construct
  REGION 1..111: note = Description of Artificial Sequence: Synthetic polypeptide
  REGION 1..111: note = 4A8 VL N95aD
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNDFGVFGGGTKLTVL

SEQ ID NO: 92 - protein, 111 aa, synthetic construct
  REGION 1..111: note = Description of Artificial Sequence: Synthetic polypeptide
  REGION 1..111: note = 4A8 VL N95aH
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNHFGVFGGGTKLTVL

SEQ ID NO: 93 - protein, 111 aa, synthetic construct
  REGION 1..111: note = Description of Artificial Sequence: Synthetic polypeptide
  REGION 1..111: note = 4A8 VL N95aK
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNKFGVFGGGTKLTVL

SEQ ID NO: 94 - protein, 111 aa, synthetic construct
  REGION 1..111: note = Description of Artificial Sequence: Synthetic polypeptide
  REGION 1..111: note = 4A8 VL N95aL
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNLFGVFGGGTKLTVL

SEQ ID NO: 95 - protein, 111 aa, synthetic construct
  REGION 1..111: note = Description of Artificial Sequence: Synthetic polypeptide
  REGION 1..111: note = 4A8 VL N95aY
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNYFGVFGGGTKLTVL

SEQ ID NO: 96 - protein, 111 aa, synthetic construct
  REGION 1..111: note = Description of Artificial Sequence: Synthetic polypeptide
  REGION 1..111: note = 4A8 VL V97A
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNNFGAFGGGTKLTVL

SEQ ID NO: 97 - protein, 111 aa, synthetic construct
  REGION 1..111: note = Description of Artificial Sequence: Synthetic polypeptide
  REGION 1..111: note = 4A8 VL V97K
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNNFGKFGGGTKLTVL

SEQ ID NO: 98 - protein, 113 aa, synthetic construct
  REGION 1..113: note = Description of Artificial Sequence: Synthetic polypeptide
  REGION 1..113: note = 4A8 VH Variant 1 (M34Q_N56S)
EVQLLESGGGLVQPGGSLRLSCAASGFTFSSSTQSWVRQAPGKGLEWVSAISGRGRSTYYADSVKGRFTISRDNSRNTLYLQMNSLRAEDTAVYYCAIQMGYWGQGILVTVSS

SEQ ID NO: 99 - protein, 113 aa, synthetic construct
  REGION 1..113: note = Description of Artificial Sequence: Synthetic polypeptide
  REGION 1..113: note = 4A8 VH Variant 2 (M34Q_A40P_N56S)
EVQLLESGGGLVQPGGSLRLSCAASGFTFSSSTQSWVRQPPGKGLEWVSAISGRGRSTYYADSVKGRFTISRDNSRNTLYLQMNSLRAEDTAVYYCAIQMGYWGQGILVTVSS

SEQ ID NO: 100 - protein, 113 aa, synthetic construct
  REGION 1..113: note = Description of Artificial Sequence: Synthetic polypeptide
  REGION 1..113: note = 4A8 VH Variant 3 (M34Q_A40P_N56S_R75K)
EVQLLESGGGLVQPGGSLRLSCAASGFTFSSSTQSWVRQPPGKGLEWVSAISGRGRSTYYADSVKGRFTISRDNSKNTLYLQMNSLRAEDTAVYYCAIQMGYWGQGILVTVSS

SEQ ID NO: 101 - protein, 113 aa, synthetic construct
  REGION 1..113: note = Description of Artificial Sequence: Synthetic polypeptide
  REGION 1..113: note = 4A8 VH Variant 4 (M34Q_A40P_N56S_M96K)
EVQLLESGGGLVQPGGSLRLSCAASGFTFSSSTQSWVRQPPGKGLEWVSAISGRGRSTYYADSVKGRFTISRDNSRNTLYLQMNSLRAEDTAVYYCAIQKGYWGQGILVTVSS

SEQ ID NO: 102 - protein, 113 aa, synthetic construct
  REGION 1..113: note = Description of Artificial Sequence: Synthetic polypeptide
  REGION 1..113: note = 4A8 VH Variant 5 (M34Q_A40P_N56S_R75K_M96K)
EVQLLESGGGLVQPGGSLRLSCAASGFTFSSSTQSWVRQPPGKGLEWVSAISGRGRSTYYADSVKGRFTISRDNSKNTLYLQMNSLRAEDTAVYYCAIQKGYWGQGILVTVSS

SEQ ID NO: 103 - protein, 113 aa, synthetic construct
  REGION 1..113: note = Description of Artificial Sequence: Synthetic polypeptide
  REGION 1..113: note = 4A8 VH Variant 6 (M34Q_A40P_N56S_R75K_I94K_M96K)
EVQLLESGGGLVQPGGSLRLSCAASGFTFSSSTQSWVRQPPGKGLEWVSAISGRGRSTYYADSVKGRFTISRDNSKNTLYLQMNSLRAEDTAVYYCAKQKGYWGQGILVTVSS

SEQ ID NO: 104 - protein, 113 aa, synthetic construct
  REGION 1..113: note = Description of Artificial Sequence: Synthetic polypeptide
  REGION 1..113: note = 4A8 VH Variant 7 (M34Q_A40P_N56S_I94K_M96K)
EVQLLESGGGLVQPGGSLRLSCAASGFTFSSSTQSWVRQPPGKGLEWVSAISGRGRSTYYADSVKGRFTISRDNSRNTLYLQMNSLRAEDTAVYYCAKQKGYWGQGILVTVSS

SEQ ID NO: 105 - protein, 113 aa, synthetic construct
  REGION 1..113: note = Description of Artificial Sequence: Synthetic polypeptide
  REGION 1..113: note = 4A8 VH Variant 8 (M34Q_N56S_M96K)
EVQLLESGGGLVQPGGSLRLSCAASGFTFSSSTQSWVRQAPGKGLEWVSAISGRGRSTYYADSVKGRFTISRDNSRNTLYLQMNSLRAEDTAVYYCAIQKGYWGQGILVTVSS

SEQ ID NO: 106 - protein, 113 aa, synthetic construct
  REGION 1..113: note = Description of Artificial Sequence: Synthetic polypeptide
  REGION 1..113: note = 4A8 VH Variant 9 (M34Q_N56S_R75K_M96K)
EVQLLESGGGLVQPGGSLRLSCAASGFTFSSSTQSWVRQAPGKGLEWVSAISGRGRSTYYADSVKGRFTISRDNSKNTLYLQMNSLRAEDTAVYYCAIQKGYWGQGILVTVSS

SEQ ID NO: 107 - protein, 113 aa, synthetic construct
  REGION 1..113: note = Description of Artificial Sequence: Synthetic polypeptide
  REGION 1..113: note = 4A8 VH Variant 10 I94K_M96K
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAKQKGYWGQGILVTVSS

SEQ ID NO: 108 - protein, 113 aa, synthetic construct
  REGION 1..113: note = Description of Artificial Sequence: Synthetic polypeptide
  REGION 1..113: note = 4A8 VH Variant 101 M34Q_A40P_N56S_R75K_M96A
EVQLLESGGGLVQPGGSLRLSCAASGFTFSSSTQSWVRQPPGKGLEWVSAISGRGRSTYYADSVKGRFTISRDNSKNTLYLQMNSLRAEDTAVYYCAIQAGYWGQGILVTVSS

SEQ ID NO: 109 - protein, 111 aa, synthetic construct
  REGION 1..111: note = Description of Artificial Sequence: Synthetic polypeptide
  REGION 1..111: note = 4A8 VL variant b N52S_N94S
QSALTQPPSASGSPGQSVTISCIGTSSDVGGYNYVSWYQQHPDKAPKLMIYEVSKRPSGVPDRFSGSKSGNTASLTVSGLQAEDEADYYCSSYAGSNNFGVFGGGTKLTVL

SEQ ID NO: 110 - protein, 111 aa, synthetic construct
  REGION 1..111: note = Description of Artificial Sequence: Synthetic polypeptide
  REGION 1..111: note = 4A8 VL variant c D41G_N52S_N94S
QSALTQPPSASGSPGQSVTISCIGTSSDVGGYNYVSWYQQHPGKAPKLMIYEVSKRPSGVPDRFSGSKSGNTASLTVSGLQAEDEADYYCSSYAGSNNFGVFGGGTKLTVL

SEQ ID NO: 111 - DNA, 339 bp, synthetic construct
  misc_feature 1..339: note = Description of Artificial Sequence: Synthetic polynucleotide
  misc_feature 1..339: note = 4A8 VH M34Q_A40P_N56S_R75K_M96A
  source 1..339: mol_type = other DNA
gaagttcagctgcttgaatctggcggaggactggttcagcctggcggatctctgagactgtcttgtgccgccagcggcttcacctttagcagcagcacacagagctgggtccgacagcctcctggcaaaggactggaatgggtgtccgccatctctggcagaggcagaagcacctactacgccgactctgtgaagggcagattcaccatcagccgggacaacagcaagaacaccctgtacctgcagatgaacagcctgagagccgaggacaccgccgtgtactattgtgccatccaggccggctattggggccagggaatactcgtgacagtgtcctca

SEQ ID NO: 112 - DNA, 333 bp, synthetic construct
  misc_feature 1..333: note = Description of Artificial Sequence: Synthetic polynucleotide
  misc_feature 1..333: note = 4A8 VL D41G_N52S_N94S
  source 1..333: mol_type = other DNA
cagtctgctctgacacagcctcctagcgcctctggctctcctggccagagcgtgaccatcagctgtatcggcaccagcagcgacgtgggcggctacaactacgtgtcctggtatcagcagcaccccggtaaggcccccaagctgatgatctacgaagtgtccaagcggcccagcggcgtgcccgatagattcagcggcagcaagagcggcaacaccgccagcctcacagtgtctggactgcaggccgaggacgaggccgactactactgtagcagctacgccggcagcaacaacttcggcgtgttcggcggaggcaccaagctgacagtccta

SEQ ID NO: 113 - protein, 436 aa, synthetic construct
  REGION 1..436: note = Description of Artificial Sequence: Synthetic polypeptide
  REGION 1..436: note = BKO-4A8-mIgG1 VH
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPG

SEQ ID NO: 114 - protein, 217 aa, synthetic construct
  REGION 1..217: note = Description of Artificial Sequence: Synthetic polypeptide
  REGION 1..217: note = BKO-4A8-mIgG1 VL
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNNFGVFGGGTKLTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 115 - protein, 439 aa, synthetic construct
  REGION 1..439: note = Description of Artificial Sequence: Synthetic polypeptide
  REGION 1..439: note = BKO-4A8 IgG4
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 116 - protein, 326 aa, synthetic construct
  REGION 1..326: note = Description of Artificial Sequence: Synthetic polypeptide
  REGION 1..326: note = IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 117 - protein, 439 aa, synthetic construct
  REGION 1..439: note = Description of Artificial Sequence: Synthetic polypeptide
  REGION 1..439: note = BKO-4A8 IgG4
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 118 - protein, 326 aa, synthetic construct
  REGION 1..326: note = Description of Artificial Sequence: Synthetic polypeptide
  REGION 1..326: note = IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 119 - protein, 438 aa, synthetic construct
  REGION 1..438: note = Description of Artificial Sequence: Synthetic polypeptide
  REGION 1..438: note = BKO-4A8 IgG2
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 120 - protein, 325 aa, synthetic construct
  REGION 1..325: note = Description of Artificial Sequence: Synthetic polypeptide
  REGION 1..325: note = IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 121 - protein, 442 aa, synthetic construct
  REGION 1..442: note = Description of Artificial Sequence: Synthetic polypeptide
  REGION 1..442: note = BKO-4A8 IgG1
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 329 aa, synthetic construct
  REGION 1..329: note = Description of Artificial Sequence: Synthetic polypeptide
  REGION 1..329: note = IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 442 aa, synthetic construct
  REGION 1..442: note = Description of Artificial Sequence: Synthetic polypeptide
  REGION 1..442: note = BKO-4A8 IgG1
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGYWGQGILVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 329 aa, synthetic construct
  REGION 1..329: note = Description of Artificial Sequence: Synthetic polypeptide
  REGION 1..329: note = IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 360 aa, synthetic construct
  REGION 1..360: note = Description of Artificial Sequence: Synthetic polypeptide
  REGION 1..360: note = Human CXCR2
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 126 - protein, 359 aa, synthetic construct
  REGION 1..359: note = Description of Artificial Sequence: Synthetic polypeptide
  REGION 1..359: note = Mouse CXCR2
MGEFKVDKFNIEDFFSGDLDIFNYSSGMPSILPDAVPCHSENLEINSYAVVVIYVLVTLLSLVGNSLVMLVILYNRSTCSVTDVYLLNLAIADLFFALTLPVWAASKVNGWTFGSTLCKIFSYVKEVTFYSSVLLLACISMDRYLAIVHATSTLIQKRHLVKFVCIAMWLLSVILALPILILRNPVKVNLSTLVCYEDVGNNTSRLRVVLRILPQTFGFLVPLLIMLFCYGFTLRTLFKAHMGQKHRAMRVIFAVVLVFLLCWLPYNLVLFTDTLMRTKLIKETCERRDDIDKALNATEILGFLHSCLNPIIYAFIGQKFRHGLLKIMATYGLVSKEFLAKEGRPSFVSSSSANTSTTL

SEQ ID NO: 127 - protein, 355 aa, synthetic construct
  REGION 1..355: note = Description of Artificial Sequence: Synthetic polypeptide
  REGION 1..355: note = cynomolgus CXCR2
MQSFNFEDFWENEDFSNYSYSSDLPPSLPDVAPCRPESLEINKYFVVIIYALVFLLSLLGNSLVMLVILHSRVGRSITDVYLLNLAMADLLFALTLPIWAAAKVNGWIFGTFLCKVVSLLKEVNFYSGILLLACISVDRYLAIVHATRTLTQKRYLVKFVCLSIWSLSLLLALPVLLFRRTVYLTYISPVCYEDMGNNTAKWRMVLRILPQTFGFILPLLIMLFCYGFTLRTLFKAHMGQKHRAMRVIFAVVLIFLLCWLPYHLVLLADTLMRTRLINETCQRRNNIDQALDATEILGILHSCLNPLIYAFIGQKFRHGLLKILATHGLISKDSLPKDSRPSFVGSSSGHTSTTL

SEQ ID NO: 128 - protein, 368 aa, synthetic construct
  REGION 1..368: note = Description of Artificial Sequence: Synthetic polypeptide
  REGION 1..368: note = human CXCR3
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 129 - protein, 352 aa, synthetic construct
  REGION 1..352: note = Description of Artificial Sequence: Synthetic polypeptide
  REGION 1..352: note = human CXCR4
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 130 - protein, 372 aa, synthetic construct
  REGION 1..372: note = Description of Artificial Sequence: Synthetic polypeptide
  REGION 1..372: note = human CXCR5
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 131 - protein, 342 aa, synthetic construct
  REGION 1..342: note = Description of Artificial Sequence: Synthetic polypeptide
  REGION 1..342: note = human CXCR6
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 132 - protein, 362 aa, synthetic construct
  REGION 1..362: note = Description of Artificial Sequence: Synthetic polypeptide
  REGION 1..362: note = human CXCR7
MDLHLFDYSEPGNFSDISWPCNSSDCIVVDTVMCPNMPNKSVLLYTLSFIYIFIFVIGMIANSVVVWVNIQAKTTGYDTHCYILNLAIADLWVVLTIPVWVVSLVQHNQWPMGELTCKVTHLIFSINLFGSIFFLTCMSVDRYLSITYFTNTPSSRKKMVRRVVCILVWLLAFCVSLPDTYYLKTVTSASNNETYCRSFYPEHSIKEWLIGMELVSVVLGFAVPFSIIAVFYFLLARAISASSDQEKHSSRKIIFSYVVVFLVCWLPYHVAVLLDIFSILHYIPFTCRLEHALFTALHVTQCLSLVHCCVNPVLYSFINRNYRYELMKAFIFKYSAKTGLTKLIDASRVSETEYSALEQSTK

SEQ ID NO: 133 - protein, 350 aa, synthetic construct
  REGION 1..350: note = Description of Artificial Sequence: Synthetic polypeptide
  REGION 1..350: note = Human CXCR1
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 134 - protein, 106 aa, synthetic construct
  REGION 1..106: note = Description of Artificial Sequence: Synthetic polypeptide
  REGION 1..106: note = Human Lambda light chain constant region
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 135 - protein, 107 aa, synthetic construct [sequence omitted]

SEQ ID NO: 136 - protein, 113 aa, synthetic construct
  REGION 1..113: note = Description of Artificial Sequence: Synthetic polypeptide
  REGION 1..113: note = 4A8 VH S32D
EVQLLESGGGLVQPGGSLRLSCAASGFTFSSDTMSWVRQAPGKGLEWVSAISGRGRNTYYADSVKGRFTISRDNSRNTLYLQMNSLRAEDTAVYYCAIQMGYWGQGILVTVSS

SEQ ID NO: 137 - protein, 113 aa, synthetic construct
  REGION 1..113: note = Description of Artificial Sequence: Synthetic polypeptide
  REGION 1..113: note = 4A8 VH S35Q
EVQLLESGGGLVQPGGSLRLSCAASGFTFSSSTMQWVRQAPGKGLEWVSAISGRGRNTYYADSVKGRFTISRDNSRNTLYLQMNSLRAEDTAVYYCAIQMGYWGQGILVTVSS

SEQ ID NO: 138 - protein, 113 aa, synthetic construct
  REGION 1..113: note = Description of Artificial Sequence: Synthetic polypeptide
  REGION 1..113: note = 4A8 VH S35D
EVQLLESGGGLVQPGGSLRLSCAASGFTFSSSTMDWVRQAPGKGLEWVSAISGRGRNTYYADSVKGRFTISRDNSRNTLYLQMNSLRAEDTAVYYCAIQMGYWGQGILVTVSS

SEQ ID NO: 139 - protein, 113 aa, synthetic construct
  REGION 1..113: note = Description of Artificial Sequence: Synthetic polypeptide
  REGION 1..113: note = 4A8 VH S35K
EVQLLESGGGLVQPGGSLRLSCAASGFTFSSSTMKWVRQAPGKGLEWVSAISGRGRNTYYADSVKGRFTISRDNSRNTLYLQMNSLRAEDTAVYYCAIQMGYWGQGILVTVSS

SEQ ID NO: 140 - protein, 113 aa, synthetic construct
  REGION 1..113: note = Description of Artificial Sequence: Synthetic polypeptide
  REGION 1..113: note = 4A8 VH A50S
EVQLLESGGGLVQPGGSLRLSCAASGFTFSSSTMSWVRQAPGKGLEWVSSISGRGRNTYYADSVKGRFTISRDNSRNTLYLQMNSLRAEDTAVYYCAIQMGYWGQGILVTVSS

SEQ ID NO: 141 - protein, 113 aa, synthetic construct
  REGION 1..113: note = Description of Artificial Sequence: Synthetic polypeptide
  REGION 1..113: note = 4A8 VH R55Q
EVQLLESGGGLVQPGGSLRLSCAASGFTFSSSTMSWVRQAPGKGLEWVSAISGRGQNTYYADSVKGRFTISRDNSRNTLYLQMNSLRAEDTAVYYCAIQMGYWGQGILVTVSS

SEQ ID NO: 142 - protein, 113 aa, synthetic construct
  REGION 1..113: note = Description of Artificial Sequence: Synthetic polypeptide
  REGION 1..113: note = 4A8 VH R55D
EVQLLESGGGLVQPGGSLRLSCAASGFTFSSSTMSWVRQAPGKGLEWVSAISGRGDNTYYADSVKGRFTISRDNSRNTLYLQMNSLRAEDTAVYYCAIQMGYWGQGILVTVSS

SEQ ID NO: 143 - protein, 113 aa, synthetic construct
  REGION 1..113: note = Description of Artificial Sequence: Synthetic polypeptide
  REGION 1..113: note = 4A8 VH R55H
EVQLLESGGGLVQPGGSLRLSCAASGFTFSSSTMSWVRQAPGKGLEWVSAISGRGHNTYYADSVKGRFTISRDNSRNTLYLQMNSLRAEDTAVYYCAIQMGYWGQGILVTVSS

SEQ ID NO: 144 - protein, 113 aa, synthetic construct
  REGION 1..113: note = Description of Artificial Sequence: Synthetic polypeptide
  REGION 1..113: note = 4A8 VH M96S
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQSGYWGQGILVTVSS

SEQ ID NO: 145 - protein, 113 aa, synthetic construct
  REGION 1..113: note = Description of Artificial Sequence: Synthetic polypeptide
  REGION 1..113: note = 4A8 VH M96D
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQDGYWGQGILVTVSS

SEQ ID NO: 146 - protein, 113 aa, synthetic construct
  REGION 1..113: note = Description of Artificial Sequence: Synthetic polypeptide
  REGION 1..113: note = 4A8 VH M96H
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQHGYWGQGILVTVSS

SEQ ID NO: 147 - protein, 113 aa, synthetic construct
  REGION 1..113: note = Description of Artificial Sequence: Synthetic polypeptide
  REGION 1..113: note = 4A8 VH M96L
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQLGYWGQGILVTVSS

SEQ ID NO: 148 - protein, 113 aa, synthetic construct
  REGION 1..113: note = Description of Artificial Sequence: Synthetic polypeptide
  REGION 1..113: note = 4A8 VH M96W
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQWGYWGQGILVTVSS

SEQ ID NO: 149 - protein, 113 aa, synthetic construct
  REGION 1..113: note = Description of Artificial Sequence: Synthetic polypeptide
  REGION 1..113: note = 4A8 VH M96Y
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQYGYWGQGILVTVSS

SEQ ID NO: 150 - protein, 113 aa, synthetic construct
  REGION 1..113: note = Description of Artificial Sequence: Synthetic polypeptide
  REGION 1..113: note = 4A8 VH Y102Q
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGQWGQGILVTVSS

SEQ ID NO: 151 - protein, 113 aa, synthetic construct
  REGION 1..113: note = Description of Artificial Sequence: Synthetic polypeptide
  REGION 1..113: note = 4A8 VH Y102D
EVQLLESGGGLVQPGGSLRLSCAASGFTFSSSTMSWVRQAPGKGLEWVSAISGRGRNTYYADSVKGRFTISRDNSRNTLYLQMNSLRAEDTAVYYCAIQMGDWGQGILVTVSS

SEQ ID NO: 152 - protein, 111 aa, synthetic construct
  REGION 1..111: note = Description of Artificial Sequence: Synthetic polypeptide
  REGION 1..111: note = 4A8 VL V51D
QSALTQPPSASGSPGQSVTISCIGTSSDVGGYNYVSWYQQHPDKAPKLMIYEDNKRPSGVPDRFSGSKSGNTASLTVSGLQAEDEADYYCSSYAGNNNFGVFGGGTKLTVL

SEQ ID NO: 153 - protein, 111 aa, synthetic construct
  REGION 1..111: note = Description of Artificial Sequence: Synthetic polypeptide
  REGION 1..111: note = 4A8 VL V51Y
QSALTQPPSASGSPGQSVTISCIGTSSDVGGYNYVSWYQQHPDKAPKLMIYEYNKRPSGVPDRFSGSKSGNTASLTVSGLQAEDEADYYCSSYAGNNNFGVFGGGTKLTVL

SEQ ID NO: 154 - protein, 111 aa, synthetic construct
  REGION 1..111: note = Description of Artificial Sequence: Synthetic polypeptide
  REGION 1..111: note = 4A8 VL R54D
QSALTQPPSASGSPGQSVTISCIGTSSDVGGYNYVSWYQQHPDKAPKLMIYEVNKDPSGVPDRFSGSKSGNTASLTVSGLQAEDEADYYCSSYAGNNNFGVFGGGTKLTVL

SEQ ID NO: 155 - protein, 111 aa, synthetic construct
  REGION 1..111: note = Description of Artificial Sequence: Synthetic polypeptide
  REGION 1..111: note = 4A8 VL Y91S
QSALTQPPSASGSPGQSVTISCIGTSSDVGGYNYVSWYQQHPDKAPKLMIYEVNKRPSGVPDRFSGSKSGNTASLTVSGLQAEDEADYYCSSSAGNNNFGVFGGGTKLTVL

SEQ ID NO: 156 - protein, 111 aa, synthetic construct
  REGION 1..111: note = Description of Artificial Sequence: Synthetic polypeptide
  REGION 1..111: note = 4A8 VL Y91H
QSALTQPPSASGSPGQSVTISCIGTSSDVGGYNYVSWYQQHPDKAPKLMIYEVNKRPSGVPDRFSGSKSGNTASLTVSGLQAEDEADYYCSSHAGNNNFGVFGGGTKLTVL

SEQ ID NO: 157 - protein, 111 aa, synthetic construct
  REGION 1..111: note = Description of Artificial Sequence: Synthetic polypeptide
  REGION 1..111: note = 4A8 VL N94H
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGHNNFGVFGGGTKLTVL

SEQ ID NO: 158 - protein, 111 aa, synthetic construct
  REGION 1..111: note = Description of Artificial Sequence: Synthetic polypeptide
  REGION 1..111: note = 4A8 VL N95aS
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNSFGVFGGGTKLTVL

SEQ ID NO: 159 - protein, 111 aa, synthetic construct
  REGION 1..111: note = Description of Artificial Sequence: Synthetic polypeptide
  REGION 1..111: note = 4A8 VL N95aW
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNWFGVFGGGTKLTVL

SEQ ID NO: 160 - protein, 111 aa, synthetic construct
  REGION 1..111: note = Description of Artificial Sequence: Synthetic polypeptide
  REGION 1..111: note = 4A8 VL V97S
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNNFGSFGGGTKLTVL

SEQ ID NO: 161 - protein, 111 aa, synthetic construct
  REGION 1..111: note = Description of Artificial Sequence: Synthetic polypeptide
  REGION 1..111: note = 4A8 VL V97D
QSALTQPPSASGSPGQSVTISCIGTSSDVGGYNYVSWYQQHPDKAPKLMIYEVNKRPSGVPDRFSGSKSGNTASLTVSGLQAEDEADYYCSSYAGNNNFGDFGGGTKLTVL

SEQ ID NO: 162 - protein, 113 aa, synthetic construct
  REGION 1..113: note = Description of Artificial Sequence: Synthetic polypeptide
  REGION 1..113: note = 4A8 VH Variant 102 M34Q_A40P_N56S_R75K_M96E
EVQLLESGGGLVQPGGSLRLSCAASGFTFSSSTQSWVRQPPGKGLEWVSAISGRGRSTYYADSVKGRFTISRDNSKNTLYLQMNSLRAEDTAVYYCAIQEGYWGQGILVTVSS

SEQ ID NO: 163 - protein, 113 aa, synthetic construct
  REGION 1..113: note = Description of Artificial Sequence: Synthetic polypeptide
  REGION 1..113: note = 4A8 VH Variant 103 M34Q_A40P_R53S_R55G_N56S_R75K
EVQLLESGGGLVQPGGSLRLSCAASGFTFSSSTQSWVRQPPGKGLEWVSAISGSGGSTYYADSVKGRFTISRDNSKNTLYLQMNSLRAEDTAVYYCAIQMGYWGQGILVTVSS

SEQ ID NO: 164 - protein, 113 aa, synthetic construct
  REGION 1..113: note = Description of Artificial Sequence: Synthetic polypeptide
  REGION 1..113: note = 4A8 VH Variant 104 M34Q_A40P_R53S_R55G_N56S_R75K_M96K
EVQLLESGGGLVQPGGSLRLSCAASGFTFSSSTQSWVRQPPGKGLEWVSAISGSGGSTYYADSVKGRFTISRDNSKNTLYLQMNSLRAEDTAVYYCAIQKGYWGQGILVTVSS

SEQ ID NO: 165 - protein, 113 aa, synthetic construct
  REGION 1..113: note = Description of Artificial Sequence: Synthetic polypeptide
  REGION 1..113: note = 4A8 VH Variant 105 M34Q_A40P_R53S_R55G_N56S_R75K_M96A
EVQLLESGGGLVQPGGSLRLSCAASGFTFSSSTQSWVRQPPGKGLEWVSAISGSGGSTYYADSVKGRFTISRDNSKNTLYLQMNSLRAEDTAVYYCAIQAGYWGQGILVTVSS

SEQ ID NO: 166 - protein, 113 aa, synthetic construct
  REGION 1..113: note = Description of Artificial Sequence: Synthetic polypeptide
  REGION 1..113: note = 4A8 VH Variant 106 M34Q_A40P_R53S_R55G_N56S_R75K_M96E
EVQLLESGGGLVQPGGSLRLSCAASGFTFSSSTQSWVRQPPGKGLEWVSAISGSGGSTYYADSVKGRFTISRDNSKNTLYLQMNSLRAEDTAVYYCAIQEGYWGQGILVTVSS

SEQ ID NO: 167 - protein, 113 aa, synthetic construct
  REGION 1..113: note = Description of Artificial Sequence: Synthetic polypeptide
  REGION 1..113: note = 4A8 Consensus VH
  MOD_RES 32: note = S, Q, H, L, W or Y
  MOD_RES 33: note = T or A
  MOD_RES 34: note = M, Q, D, H or W
  MOD_RES 51: note = I or H
  MOD_RES 53: note = G or D
  MOD_RES 54: note = R, S or Q
  MOD_RES 55: note = G or D
  MOD_RES 57: note = N or S
  MOD_RES 98: note = I or K
  MOD_RES 100: note = M, A, Q or K
  MOD_RES 101: note = G or D
  MOD_RES 102: note = Y, S or K
EVQLLESGGGLVQPGGSLRLSCAASGFTFSSXXXSWVRQAPGKGLEWVSAXSXXXRXTYYADSVKGRFTISRDNSRNTLYLQMNSLRAEDTAVYYCAXQXXXWGQGILVTVSS

SEQ ID NO: 168 - protein, 111 aa, synthetic construct
  REGION 1..111: note = Description of Artificial Sequence: Synthetic polypeptide
  REGION 1..111: note = 4A8 Consensus VL
  MOD_RES 52: note = E or D
  MOD_RES 54: note = N, D or S
  MOD_RES 55: note = K, A, D or H
  MOD_RES 56: note = R or Q
  MOD_RES 93: note = Y or A
  MOD_RES 96: note = N, A, S, K, L, W or Y
  MOD_RES 98: note = N, Q, D, H, K, L or Y
  MOD_RES 101: note = V, A or K
QSALTQPPSASGSPGQSVTISCIGTSSDVGGYNYVSWYQQHPDKAPKLMIYXVXXXPSGVPDRFSGSKSGNTASLTVSGLQAEDEADYYCSSXAGXNXFGXFGGGTKLTVL

SEQ ID NO: 169 - skipped

SEQ ID NO: 170 - protein, 17 aa, synthetic construct
  REGION 1..17: note = Description of Artificial Sequence: Synthetic peptide
  REGION 1..17: note = Consensus VH CDR2
  MOD_RES 2: note = I or H
  MOD_RES 4: note = G or D
  MOD_RES 5: note = R, S or Q
  MOD_RES 6: note = G or D
  MOD_RES 8: note = N or S
AXSXXXRXTYYADSVKG

SEQ ID NO: 171 - skipped

SEQ ID NO: 172 - protein, 14 aa, synthetic construct
  REGION 1..14: note = Description of Artificial Sequence: Synthetic peptide
  REGION 1..14: note = Consensus VL CDR1
IGTSSDVGGYNYVS

SEQ ID NO: 173 - skipped

SEQ ID NO: 174 - protein, 11 aa, synthetic construct
  REGION 1..11: note = Description of Artificial Sequence: Synthetic peptide
  REGION 1..11: note = Consensus VL CDR3
  MOD_RES 3: note = Y or A
  MOD_RES 6: note = N, A, S, K, L, W or Y
  MOD_RES 8: note = N, Q, D, H, K, L or Y
  MOD_RES 11: note = V, A or K
SSXAGXNXFGX

SEQ ID NO: 175 - protein, 5 aa, synthetic construct
  REGION 1..5: note = Description of Artificial Sequence: Synthetic peptide
  REGION 1..5: note = 4A8 VHCDR1
SSTMS

SEQ ID NO: 176 - protein, 5 aa, synthetic construct
  REGION 1..5: note = Description of Artificial Sequence: Synthetic peptide
  REGION 1..5: note = 4A8 S32Q VHCDR1
SQTMS

SEQ ID NO: 177 - protein, 5 aa, synthetic construct
  REGION 1..5: note = Description of Artificial Sequence: Synthetic peptide
  REGION 1..5: note = 4A8 S32H VHCDR1
SHTMS

SEQ ID NO: 178 - protein, 5 aa, synthetic construct
  REGION 1..5: note = Description of Artificial Sequence: Synthetic peptide
  REGION 1..5: note = 4A8 S32L VHCDR1
SLTMS

SEQ ID NO: 179 - protein, 5 aa, synthetic construct
  REGION 1..5: note = Description of Artificial Sequence: Synthetic peptide
  REGION 1..5: note = 4A8 S32W VHCDR1
SWTMS

SEQ ID NO: 180 - protein, 5 aa, synthetic construct
  REGION 1..5: note = Description of Artificial Sequence: Synthetic peptide
  REGION 1..5: note = 4A8 S32Y VHCDR1
SYTMS

SEQ ID NO: 181 - protein, 5 aa, synthetic construct
  REGION 1..5: note = Description of Artificial Sequence: Synthetic peptide
  REGION 1..5: note = 4A8 T33A VHCDR1
SSAMS

SEQ ID NO: 182 - protein, 5 aa, synthetic construct
  REGION 1..5: note = Description of Artificial Sequence: Synthetic peptide
  REGION 1..5: note = 4A8 M34Q VHCDR1
SSTQS

SEQ ID NO: 183 - protein, 5 aa, synthetic construct
  REGION 1..5: note = Description of Artificial Sequence: Synthetic peptide
  REGION 1..5: note = 4A8 M34D VHCDR1
SSTDS

SEQ ID NO: 184 - protein, 5 aa, synthetic construct
  REGION 1..5: note = Description of Artificial Sequence: Synthetic peptide
  REGION 1..5: note = 4A8 M34H VHCDR1
SSTHS

SEQ ID NO: 185 - protein, 5 aa, synthetic construct
  REGION 1..5: note = Description of Artificial Sequence: Synthetic peptide
  REGION 1..5: note = 4A8 M34W VHCDR1
SSTWS

SEQ ID NO: 186 - protein, 17 aa, synthetic construct
  REGION 1..17: note = Description of Artificial Sequence: Synthetic peptide
  REGION 1..17: note = 4A8 VHCDR2
AISGRGRNTYYADSVKG

SEQ ID NO: 187 - protein, 17 aa, synthetic construct
  REGION 1..17: note = Description of Artificial Sequence: Synthetic peptide
  REGION 1..17: note = 4A8 I51H VHCDR2
AHSGRGRNTYYADSVKG

SEQ ID NO: 188 - protein, 17 aa, synthetic construct
  REGION 1..17: note = Description of Artificial Sequence: Synthetic peptide
  REGION 1..17: note = 4A8 G52aD VHCDR2
AISDRGRNTYYADSVKG

SEQ ID NO: 189 - protein, 17 aa, synthetic construct
  REGION 1..17: note = Description of Artificial Sequence: Synthetic peptide
  REGION 1..17: note = 4A8 R53S VHCDR2
AISGSGRNTYYADSVKG

SEQ ID NO: 190 - protein, 17 aa, synthetic construct
  REGION 1..17: note = Description of Artificial Sequence: Synthetic peptide
  REGION 1..17: note = 4A8 R53Q VHCDR2
AISGQGRNTYYADSVKG

SEQ ID NO: 191 - protein, 17 aa, synthetic construct
  REGION 1..17: note = Description of Artificial Sequence: Synthetic peptide
  REGION 1..17: note = 4A8 G54D VHCDR2
AISGRDRNTYYADSVKG

SEQ ID NO: 192 - protein, 17 aa, synthetic construct
  REGION 1..17: note = Description of Artificial Sequence: Synthetic peptide
  REGION 1..17: note = 4A8 N56S VHCDR2
AISGRGRSTYYADSVKG

SEQ ID NO: 193 - protein, 17 aa, synthetic construct
  REGION 1..17: note = Description of Artificial Sequence: Synthetic peptide
  REGION 1..17: note = 4A8 103,104, 105 VHCDR2
AISGSGGSTYYADSVKG

SEQ ID NO: 194 - protein, 4 aa, synthetic construct
  REGION 1..4: note = Description of Artificial Sequence: Synthetic peptide
  REGION 1..4: note = 4A8 VHCDR3
QMGY

SEQ ID NO: 195 - protein, 4 aa, synthetic construct
  REGION 1..4: note = Description of Artificial Sequence: Synthetic peptide
  REGION 1..4: note = 4A8 M96A VHCDR3
QAGY

SEQ ID NO: 196 - protein, 4 aa, synthetic construct
  REGION 1..4: note = Description of Artificial Sequence: Synthetic peptide
  REGION 1..4: note = 4A8 M96Q VHCDR3
QQGY

SEQ ID NO: 197 - protein, 4 aa, synthetic construct
  REGION 1..4: note = Description of Artificial Sequence: Synthetic peptide
  REGION 1..4: note = 4A8 M96K VHCDR3
QKGY

SEQ ID NO: 198 - protein, 4 aa, synthetic construct
  REGION 1..4: note = Description of Artificial Sequence: Synthetic peptide
  REGION 1..4: note = 4A8 G101D VHCDR3
QMDY

SEQ ID NO: 199 - protein, 4 aa, synthetic construct
  REGION 1..4: note = Description of Artificial Sequence: Synthetic peptide
  REGION 1..4: note = 4A8 Y102S VHCDR3
QMGS

SEQ ID NO: 200 - protein, 4 aa, synthetic construct
  REGION 1..4: note = Description of Artificial Sequence: Synthetic peptide
  REGION 1..4: note = 4A8 Y102K VHCDR3
QMGK

SEQ ID NO: 201 - protein, 14 aa, synthetic construct
  REGION 1..14: note = Description of Artificial Sequence: Synthetic peptide
  REGION 1..14: note = 4A8 VLCDR1 and Consensus VL CDR1
IGTSSDVGGYNYVS

SEQ ID NO: 202 - protein, 7 aa, synthetic construct
  REGION 1..7: note = Description of Artificial Sequence: Synthetic peptide
  REGION 1..7: note = 4A8 VLCDR2
EVNKRPS

SEQ ID NO: 203 - protein, 7 aa, synthetic construct
  REGION 1..7: note = Description of Artificial Sequence: Synthetic peptide
  REGION 1..7: note = 4A8 E50D VLCDR2
DVNKRPS

SEQ ID NO: 204 - protein, 7 aa, synthetic construct
  REGION 1..7: note = Description of Artificial Sequence: Synthetic peptide
  REGION 1..7: note = 4A8 N52D VLCDR2
EVDKRPS

SEQ ID NO: 205 - protein, 7 aa, synthetic construct
  REGION 1..7: note = Description of Artificial Sequence: Synthetic peptide
  REGION 1..7: note = 4A8 N52S VLCDR2
EVSKRPS

SEQ ID NO: 206 - protein, 7 aa, synthetic construct
  REGION 1..7: note = Description of Artificial Sequence: Synthetic peptide
  REGION 1..7: note = 4A8 K53A VLCDR2
EVNARPS

SEQ ID NO: 207 - protein, 7 aa, synthetic construct
  REGION 1..7: note = Description of Artificial Sequence: Synthetic peptide
  REGION 1..7: note = 4A8 K53D VLCDR2
EVNDRPS

SEQ ID NO: 208 - protein, 7 aa, synthetic construct
  REGION 1..7: note = Description of Artificial Sequence: Synthetic peptide
  REGION 1..7: note = 4A8 K53H VLCDR2
EVNHRPS

SEQ ID NO: 209 - protein, 7 aa, synthetic construct
  REGION 1..7: note = Description of Artificial Sequence: Synthetic peptide
  REGION 1..7: note = 4A8 R54Q VLCDR2
EVNKQPS

SEQ ID NO: 210 - protein, 11 aa, synthetic construct
  REGION 1..11: note = Description of Artificial Sequence: Synthetic peptide
  REGION 1..11: note = 4A8 VLCDR3
SSYAGNNNFGV

SEQ ID NO: 211 - protein, 11 aa, synthetic construct
  REGION 1..11: note = Description of Artificial Sequence: Synthetic peptide
  REGION 1..11: note = 4A8 Y91A VLCDR3
SSAAGNNNFGV

SEQ ID NO: 212 - protein, 11 aa, synthetic construct
  REGION 1..11: note = Description of Artificial Sequence: Synthetic peptide
  REGION 1..11: note = 4A8 N94A VLCDR3
SSYAGANNFGV

SEQ ID NO: 213 - protein, 11 aa, synthetic construct
  REGION 1..11: note = Description of Artificial Sequence: Synthetic peptide
  REGION 1..11: note = 4A8 N94S VLCDR3
SSYAGSNNFGV

SEQ ID NO: 214 - protein, 11 aa, synthetic construct
  REGION 1..11: note = Description of Artificial Sequence: Synthetic peptide
  REGION 1..11: note = 4A8 N94K VLCDR3
SSYAGKNNFGV

SEQ ID NO: 215 - protein, 11 aa, synthetic construct
  REGION 1..11: note = Description of Artificial Sequence: Synthetic peptide
  REGION 1..11: note = 4A8 N94L VLCDR3
SSYAGLNNFGV

SEQ ID NO: 216 - protein, 11 aa, synthetic construct
  REGION 1..11: note = Description of Artificial Sequence: Synthetic peptide
  REGION 1..11: note = 4A8 N94W VLCDR3
SSYAGWNNFGV

SEQ ID NO: 217 - protein, 11 aa, synthetic construct
  REGION 1..11: note = Description of Artificial Sequence: Synthetic peptide
  REGION 1..11: note = 4A8 N94Y VLCDR3
SSYAGYNNFGV

SEQ ID NO: 218 - protein, 11 aa, synthetic construct
  REGION 1..11: note = Description of Artificial Sequence: Synthetic peptide
  REGION 1..11: note = 4A8 N95aQ VLCDR3
SSYAGNNQFGV

SEQ ID NO: 219 - protein, 11 aa, synthetic construct
  REGION 1..11: note = Description of Artificial Sequence: Synthetic peptide
  REGION 1..11: note = 4A8 N95aD VLCDR3
SSYAGNNDFGV

SEQ ID NO: 220 - protein, 11 aa, synthetic construct
  REGION 1..11: note = Description of Artificial Sequence: Synthetic peptide
  REGION 1..11: note = 4A8 N95aH VLCDR3
SSYAGNNHFGV

SEQ ID NO: 221 - protein, 11 aa, synthetic construct
  REGION 1..11: note = Description of Artificial Sequence: Synthetic peptide
  REGION 1..11: note = 4A8 N95aK VLCDR3
SSYAGNNKFGV

SEQ ID NO: 222 - protein, 11 aa, synthetic construct
  REGION 1..11: note = Description of Artificial Sequence: Synthetic peptide
  REGION 1..11: note = 4A8 N95aL VLCDR3
SSYAGNNLFGV

SEQ ID NO: 223 - protein, 11 aa, synthetic construct
  REGION 1..11: note = Description of Artificial Sequence: Synthetic peptide
  REGION 1..11: note = A48 N95aYVLCDR3
SSYAGNNYFGV

SEQ ID NO: 224 - protein, 11 aa, synthetic construct
  REGION 1..11: note = Description of Artificial Sequence: Synthetic peptide
  REGION 1..11: note = 4A8 V97A VLCDR3
SSYAGNNNFGA

SEQ ID NO: 225 - protein, 11 aa, synthetic construct
  REGION 1..11: note = Description of Artificial Sequence: Synthetic peptide
  REGION 1..11: note = 4A8 V97K VLCDR3
SSYAGNNNFGK

SEQ ID NO: 226 - protein, 113 aa, synthetic construct
  REGION 1..113: note = Description of Artificial Sequence: Synthetic polypeptide
  REGION 1..113: note = 4A8 Consensus VH
  MOD_RES 34: note = M or Q
  MOD_RES 40: note = A or P
  MOD_RES 54: note = R or S
  MOD_RES 56: note = R or G
  MOD_RES 57: note = N or S
  MOD_RES 76: note = R or K
  MOD_RES 98: note = I or K
  MOD_RES 100: note = M, K or A
EVQLLESGGGLVQPGGSLRLSCAASGFTFSSSTXSWVRQXPGKGLEWVSAISGXGXXTYYADSVKGRFTISRDNSXNTLYLQMNSLRAEDTAVYYCAXQXGYWGQGILVTVSS

SEQ ID NO: 227 - protein, 111 aa, synthetic construct
  REGION 1..111: note = Description of Artificial Sequence: Synthetic polypeptide
  REGION 1..111: note = 4A8 Consensus VL
  MOD_RES 43: note = D or G
  MOD_RES 54: note = N or S
  MOD_RES 96: note = N or S
QSALTQPPSASGSPGQSVTISCIGTSSDVGGYNYVSWYQQHPXKAPKLMIYEVXKRPSGVPDRFSGSKSGNTASLTVSGLQAEDEADYYCSSYAGXNNFGVFGGGTKLTVL

SEQ ID NO: 228 - protein, 5 aa, synthetic construct
  REGION 1..5: note = Description of Artificial Sequence: Synthetic peptide
  REGION 1..5: note = Consensus VH CDR1
  MOD_RES 4: note = M or Q
SSTXS

SEQ ID NO: 229 - protein, 17 aa, synthetic construct
  REGION 1..17: note = Description of Artificial Sequence: Synthetic peptide
  REGION 1..17: note = Consensus VH CDR2
  MOD_RES 5: note = R or S
  MOD_RES 7: note = R or G
  MOD_RES 8: note = N or S
AISGXGXXTYYADSVKG

SEQ ID NO: 230 - skipped

SEQ ID NO: 231 - protein, 7 aa, synthetic construct
  REGION 1..7: note = Description of Artificial Sequence: Synthetic peptide
  REGION 1..7: note = Consensus VL CDR2
  MOD_RES 3: note = N or S
EVXKRPS

SEQ ID NO: 232 - protein, 11 aa, synthetic construct
  REGION 1..11: note = Description of Artificial Sequence: Synthetic peptide
  REGION 1..11: note = Consensus VL CDR3
  MOD_RES 6: note = N or S
SSYAGXNNFGV

SEQ ID NO: 233 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = BKO-4A8 VH
  source 1..339: mol_type = other DNA
gaagtgcagctgctggaatctggcggaggactggtgcagcctggcggcagcctgagactgtcttgtgccgccagcggcttcaccttcagcagcagcacaatgagctgggtccgacaggcccctggcaagggactggaatgggtgtccgccatcagcggcagaggccggaacacctactacgccgacagcgtgaagggccggttcaccatcagccgggacaacagcagaaacaccctgtacctgcagatgaacagcctgcgggccgaggacaccgccgtgtactactgtgccatccagatgggctactggggccagggcattctcgtgacagtgtcctca

SEQ ID NO: 234 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 1
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacaggcccctggcaagggactggaatgggtgtccgccatcagcggcagaggccggagtacctactacgccgacagcgtgaagggccggttcaccatcagccgggacaacagcagaaacaccctgtacctgcagatgaacagcctgcgggccgaggacaccgccgtgtactactgtgccatccagatgggctactggggccagggcattctcgtgacagtgtcctca

SEQ ID NO: 235 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 2
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacagcctcctggcaagggactggaatgggtgtccgccatcagcggcagaggccggagtacctactacgccgacagcgtgaagggccggttcaccatcagccgggacaacagcagaaacaccctgtacctgcagatgaacagcctgcgggccgaggacaccgccgtgtactactgtgccatccagatgggctactggggccagggcattctcgtgacagtgtcctca

SEQ ID NO: 236 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 3
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacagcctcctggcaagggactggaatgggtgtccgccatcagcggcagaggccggagtacctactacgccgacagcgtgaagggccggttcaccatcagccgggacaacagcaaaaacaccctgtacctgcagatgaacagcctgcgggccgaggacaccgccgtgtactactgtgccatccagatgggctactggggccagggcattctcgtgacagtgtcctca

SEQ ID NO: 237 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 4
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacagcctcctggcaagggactggaatgggtgtccgccatcagcggcagaggccggagtacctactacgccgacagcgtgaagggccggttcaccatcagccgggacaacagcagaaacaccctgtacctgcagatgaacagcctgcgggccgaggacaccgccgtgtactactgtgccatccagaagggctactggggccagggcattctcgtgacagtgtcctca

SEQ ID NO: 238 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 5
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacagcctcctggcaagggactggaatgggtgtccgccatcagcggcagaggccggagtacctactacgccgacagcgtgaagggccggttcaccatcagccgggacaacagcaaaaacaccctgtacctgcagatgaacagcctgcgggccgaggacaccgccgtgtactactgtgccatccagaagggctactggggccagggcattctcgtgacagtgtcctca

SEQ ID NO: 239 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 6
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacagcctcctggcaagggactggaatgggtgtccgccatcagcggcagaggccggagtacctactacgccgacagcgtgaagggccggttcaccatcagccgggacaacagcaaaaacaccctgtacctgcagatgaacagcctgcgggccgaggacaccgccgtgtactactgtgccaagcagaagggctactggggccagggcattctcgtgacagtgtcctca

SEQ ID NO: 240 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 7
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacagcctcctggcaagggactggaatgggtgtccgccatcagcggcagaggccggagtacctactacgccgacagcgtgaagggccggttcaccatcagccgggacaacagcagaaacaccctgtacctgcagatgaacagcctgcgggccgaggacaccgccgtgtactactgtgccaagcagaagggctactggggccagggcattctcgtgacagtgtcctca

SEQ ID NO: 241 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 8
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacaggcccctggcaagggactggaatgggtgtccgccatcagcggcagaggccggagtacctactacgccgacagcgtgaagggccggttcaccatcagccgggacaacagcagaaacaccctgtacctgcagatgaacagcctgcgggccgaggacaccgccgtgtactactgtgccatccagaagggctactggggccagggcattctcgtgacagtgtcctca

SEQ ID NO: 242 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 9
  source 1..339: mol_type = other DNA
gaagtgcagctgctggaatctggcggaggactggtgcagcctggcggcagcctgagactgtcttgtgccgccagcggcttcaccttcagcagcagcacacagagctgggtccgacaggcccctggcaagggactggaatgggtgtccgccatcagcggcagaggccggagtacctactacgccgacagcgtgaagggccggttcaccatcagccgggacaacagcaaaaacaccctgtacctgcagatgaacagcctgcgggccgaggacaccgccgtgtactactgtgccatccagaagggctactggggccagggcattctcgtgacagtgtcctca

SEQ ID NO: 243 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 10
  source 1..339: mol_type = other DNA
gaagtgcagctgctggaatctggcggaggactggtgcagcctggcggcagcctgagactgtcttgtgccgccagcggcttcaccttcagcagcagcacaatgagctgggtccgacaggcccctggcaagggactggaatgggtgtccgccatcagcggcagaggccggaacacctactacgccgacagcgtgaagggccggttcaccatcagccgggacaacagcagaaacaccctgtacctgcagatgaacagcctgcgggccgaggacaccgccgtgtactactgtgccaagcagaagggctactggggccagggcattctcgtgacagtgtcctca

SEQ ID NO: 244 - DNA, 339 bp, synthetic construct; identical to SEQ ID NO: 111
  misc_feature 1..339: note = Description of Artificial Sequence: Synthetic polynucleotide
  misc_feature 1..339: note = Completely synthesized
  misc_feature 1..339: note = 4A8 VH Variant 101
  source 1..339: mol_type = other DNA

SEQ ID NO: 245 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 103
  source 1..339: mol_type = other DNA
gaagttcagctgcttgaatctggcggaggactggttcagcctggcggatctctgagactgtcttgtgccgccagcggcttcacctttagcagcagcacacagagctgggtccgacagcctcctggcaaaggactggaatgggtgtccgccatctctggcagcggcggcagcacatattacgccgattctgtgaagggcagattcaccatcagccgggacaacagcaagaacaccctgtacctgcagatgaacagcctgagagccgaggacaccgccgtgtactattgcgccatccagatgggctattggggccagggaatcctcgtgacagtgtcctca

SEQ ID NO: 246 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 104
  source 1..339: mol_type = other DNA
gaagttcagctgcttgaatctggcggaggactggttcagcctggcggatctctgagactgtcttgtgccgccagcggcttcacctttagcagcagcacacagagctgggtccgacagcctcctggcaaaggactggaatgggtgtccgccatctctggcagcggcggcagcacatattacgccgattctgtgaagggcagattcaccatcagccgggacaacagcaagaacaccctgtacctgcagatgaacagcctgagagccgaggacaccgccgtgtactattgcgccatccagaaaggctattggggccagggcatcctcgtgacagtgtcctca

SEQ ID NO: 247 - DNA, 339 bp, synthetic construct
  misc_feature 1..339: note = Description of Artificial Sequence: Synthetic polynucleotide
  misc_feature 1..339: note = Completely synthesized
  misc_feature 1..339: note = 4A8 VH Variant 105
  source 1..339: mol_type = other DNA
gaagttcagctgcttgaatctggcggaggactggttcagcctggcggatctctgagactgtcttgtgccgccagcggcttcacctttagcagcagcacacagagctgggtccgacagcctcctggcaaaggactggaatgggtgtccgccatctctggcagcggcggcagcacatattacgccgattctgtgaagggcagattcaccatcagccgggacaacagcaagaacaccctgtacctgcagatgaacagcctgagagccgaggacaccgccgtgtactattgtgccatccaggccggctattggggccagggaatactcgtgacagtgtcctca

SEQ ID NO: 248 - DNA, 987 bp, Homo sapiens
  misc_feature 1..987: note = Human IgG1
  source 1..987: mol_type = genomic DNA
gctagcaccaagggacccagcgtgttccccctggcccccagcagcaagagcacatctggcggaacagccgccctgggctgcctggtgaaagactacttccccgagcccgtgaccgtgagctggaacagcggagccctgaccagcggcgtgcacacctttccagccgtgctgcagagcagcggcctgtacagcctgagcagcgtggtgacagtgccctctagcagcctgggcacccagacctacatctgcaacgtgaaccacaagcccagcaacaccaaggtggacaaaaaggtggaacccaagagctgcgacaagacccacacctgtcccccctgccctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgctcccatcgagaaaaccatcagcaaggccaagggccagccccgcgagcctcaggtgtacacactgccccccagccgggacgagctgaccaagaaccaggtgtccctgacctgtctggtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggc

SEQ ID NO: 249 - DNA, 987 bp, synthetic construct
  misc_feature 1..987: note = Description of Artificial Sequence: Synthetic polynucleotide
  misc_feature 1..987: note = Completely synthesized
  misc_feature 1..987: note = Human IgG1
  source 1..987: mol_type = other DNA
gctagcaccaagggacccagcgtgttccccctggcccccagcagcaagagcacatctggcggaacagccgccctgggctgcctggtgaaagactacttccccgagcccgtgaccgtgagctggaacagcggagccctgaccagcggcgtgcacacctttccagccgtgctgcagagcagcggcctgtacagcctgagcagcgtggtgacagtgccctctagcagcctgggcacccagacctacatctgcaacgtgaaccacaagcccagcaacaccaaggtggacaaaaaggtggaacccaagagctgcgacaagacccacacctgtcccccctgccctgcccctgaactggctggcgctccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgctcccatcgagaaaaccatcagcaaggccaagggccagccccgcgagcctcaggtgtacacactgccccccagccgggacgagctgaccaagaaccaggtgtccctgacctgtctggtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggc

SEQ ID NO: 250 - DNA, 978 bp, Homo sapiens
  misc_feature 1..978: note = Human IgG4
  source 1..978: mol_type = genomic DNA
gctagcaccaagggccccagcgtgttccccctggccccttgtagcagaagcaccagcgagagcacagccgccctgggctgcctggtgaaagactacttccccgagcccgtcaccgtgtcctggaacagcggagccctgaccagcggcgtgcacacctttccagccgtgctgcagagcagcggcctgtacagcctgagcagcgtggtgacagtgccctccagcagcctgggcaccaagacctacacctgtaacgtggaccacaagcccagcaacaccaaggtggacaagcgggtggaatctaagtacggcccaccctgccccccctgccctgcccctgaatttctgggcggaccctccgtgttcctgttccccccaaagcccaaggacaccctgatgatcagccggacccccgaagtgacctgcgtggtggtggacgtgtcccaggaagatcccgaggtccagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaagtctccaacaagggcctgcccagctccatcgagaaaaccatcagcaaggccaagggccagccccgcgagcctcaggtgtacacactgccccccagccaggaagagatgaccaagaaccaggtgtccctgacctgtctggtgaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggc

SEQ ID NO: 251 - DNA, 978 bp, synthetic construct
  misc_feature 1..978: note = Description of Artificial Sequence: Synthetic polynucleotide
  misc_feature 1..978: note = Completely synthesized
  misc_feature 1..978: note = Human IgG4
  source 1..978: mol_type = other DNA
gctagcaccaagggccccagcgtgttccccctggccccttgtagcagaagcaccagcgagagcacagccgccctgggctgcctggtgaaagactacttccccgagcccgtcaccgtgtcctggaacagcggagccctgaccagcggcgtgcacacctttccagccgtgctgcagagcagcggcctgtacagcctgagcagcgtggtgacagtgccctccagcagcctgggcaccaagacctacacctgtaacgtggaccacaagcccagcaacaccaaggtggacaagcgggtggaatctaagtacggcccaccctgccccccctgccctgcccctgaatttctgggcggaccctccgtgttcctgttccccccaaagcccaaggacaccctgtatatcactcgggagcccgaagtgacctgcgtggtggtggacgtgtcccaggaagatcccgaggtccagttcaattggtacgtggacggcgtggaagtgcacaacgccaagaccaagcccagagaggaacagttcaacagcacctaccgggtggtgtccgtgctgaccgtgctgcaccaggactggctgaacggcaaagagtacaagtgcaaagtctccaacaagggcctgcccagctccatcgagaaaaccatcagcaaggccaagggccagccccgcgagcctcaggtgtacacactgccccccagccaggaagagatgaccaagaaccaggtgtccctgacctgtctggtgaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggc

SEQ ID NO: 252 - DNA, 975 bp, synthetic construct
  misc_feature 1..975: note = Description of Artificial Sequence: Synthetic polynucleotide
  misc_feature 1..975: note = Completely synthesized
  misc_feature 1..975: note = Human IgG2
  source 1..975: mol_type = other DNA
gctagcaccaagggccccagcgtgttccctctggccccttgtagcagaagcaccagcgagtctacagccgccctgggctgcctcgtgaaggactactttcccgagcccgtcaccgtgtcctggaactctggcgctctgacaagcggcgtgcacacctttccagccgtgctgcagagcagcggcctgtactctctgagcagcgtcgtgaccgtgcccagcagcaatttcggcacccagacctacacctgtaacgtggaccacaagcccagcaacaccaaggtggacaagaccgtggaacggaagtgctgcgtggaatgccccccttgtcctgcccctccagtggctggcccttccgtgttcctgttccccccaaagcccaaggacaccctgatgatcagccggacccccgaagtgacctgcgtggtggtggatgtgtcccacgaggaccccgaggtgcagttcaattggtacgtggacggcgtggaagtgcacaacgccaagaccaagcccagagaggaacagttcaacagcaccttccgggtggtgtccgtgctgaccgtggtgcatcaggactggctgaacggcaaagagtacaagtgcaaggtgtccaacaagggcctgcccagctccatcgagaaaaccatcagcaagaccaaaggccagccccgcgagccccaggtgtacacactgcctccaagccgggaagagatgaccaagaatcaggtgtccctgacctgtctcgtgaaaggcttctacccctccgatatcgccgtggaatgggagagcaacggccagcccgagaacaactacaagaccaccccccccatgctggacagcgacggctcattcttcctgtacagcaagctgacagtggacaagtcccggtggcagcagggcaacgtgttcagctgcagcgtgatgcacgaggccctgcacaaccactacacccagaagtccctgagcctgagccctggc

SEQ ID NO: 253 - DNA, 333 bp, synthetic construct
  misc_feature 1..333: note = Description of Artificial Sequence: Synthetic polynucleotide
  misc_feature 1..333: note = Completely synthesized
  misc_feature 1..333: note = BKO-4A8 VL
  source 1..333: mol_type = other DNA
cagtctgctctgacacagcctcctagcgcctctggctctcctggccagagcgtgaccatcagctgtatcggcaccagcagcgacgtgggcggctacaactacgtgtcctggtatcagcagcaccccgacaaggcccccaagctgatgatctacgaagtgaacaagcggcccagcggcgtgcccgatagattcagcggcagcaagagcggcaacaccgccagcctcacagtgtctggactgcaggccgaggacgaggccgactactactgtagcagctacgccggcaacaacaacttcggcgtgttcggcggaggcaccaagctgacagtccta

SEQ ID NO: 254 - DNA, 333 bp, synthetic construct
  misc_feature 1..333: note = Description of Artificial Sequence: Synthetic polynucleotide
  misc_feature 1..333: note = Completely synthesized
  misc_feature 1..333: note = 4A8 VL variant b
  source 1..333: mol_type = other DNA
cagtctgctctgacacagcctcctagcgcctctggctctcctggccagagcgtgaccatcagctgtatcggcaccagcagcgacgtgggcggctacaactacgtgtcctggtatcagcagcaccccgacaaggcccccaagctgatgatctacgaagtgtccaagcggcccagcggcgtgcccgatagattcagcggcagcaagagcggcaacaccgccagcctcacagtgtctggactgcaggccgaggacgaggccgactactactgtagcagctacgccggcagcaacaacttcggcgtgttcggcggaggcaccaagctgacagtccta

SEQ ID NO: 255 - DNA, 333 bp, synthetic construct; identical to SEQ ID NO: 112
  misc_feature 1..333: note = Description of Artificial Sequence: Synthetic polynucleotide
  misc_feature 1..333: note = Completely synthesized
  misc_feature 1..333: note = 4A8 VL variant c
  source 1..333: mol_type = other DNA

SEQ ID NO: 256 - DNA, 318 bp, Homo sapiens
  misc_feature 1..318: note = lambda constant light chain
  source 1..318: mol_type = genomic DNA
ggtcagcccaaggccgctcccagcgtgaccctgttccccccaagcagcgaggaactgcaggccaacaaggccaccctggtgtgcctgatcagcgacttctaccctggggccgtgaccgtggcctggaaggccgatagcagccctgtgaaggccggcgtggaaaccaccaccccctccaagcagagcaacaacaaatacgccgccagcagctacctgtccctgacccccgagcagtggaagtcccaccggtcctacagctgccaggtgacacacgagggcagcaccgtggaaaagaccgtggcccccaccgagtgcagc

(19) United States
(12) Patent Application Publication
(10) Pub. No.: US 2024/0301076 A1
(43) Pub. Date: Sep. 12, 2024

(54) ANTI-CXCR2 ANTIBODIES AND USES THEREOF

(71) Applicant: Cephalon LLC, West Chester, PA (US)

(72) Inventors: Doris Shim Siew Chen, Yagoona (AU); Lynn Dorothy Poulton, Macquarie Park (AU); Adam Clarke, Macquarie Park (AU); David Jose Simon Laine, Macquarie Park (AU); Matthew Pollard, Macquarie Park (AU); Bridget Ann Cooksey, Macquarie Park (AU); Anthony Doyle, Macquarie Park (AU); Jason William Gill, Macquarie Park (AU)

(21) Appl. No.: 18/666,190

(22) Filed: May 16, 2024

Related U.S. Application Data
(63) Continuation of application No. 17/718,622, filed on Apr. 12, 2022, now Pat. No. 12,018,084.
Application No. 17/718,622 is a continuation of application No. 16/527,354, filed on Jul. 31, 2019, now Pat. No. 11,332,534.
(60) Provisional application No. 62/713,095, filed on Aug. 1, 2018.

(51) Int. Cl. C07K 16/28; A61K 39/00; A61K 39/395; A61P 11/00; G01N 33/53

(52) U.S. Cl. CPC C07K 16/2866; A61P 11/00; A61K 39/395; A61K 2039/505; C07K 2317/21; C07K 2317/76; C07K 2317/92; G01N 33/53

(57) ABSTRACT

Disclosed herein are human antibody molecules that immunospecifically bind to human CXCR2. The disclosed human antibody molecules are potent and selective antagonists of CXCR2 functions and prevent the recruitment of neutrophils into tissues without strongly depleting circulating neutrophil numbers. Pharmaceutical compositions, nucleic acid molecules, vectors, cells, and uses of the disclosed antibodies are also provided.

### CROSS REFERENCE TO RELATED APPLICATIONS

This application is a continuation of U.S. application Ser. No. 17/718,622, filed on Apr. 12, 2022, which is a continuation of U.S. application Ser. No. 16/527,354, filed on Jul. 31, 2019 (U.S. Pat. No. 11,332,534), which claims the benefit of the filing date of U.S. Provisional Application No. 62/713,095, filed on Aug. 1, 2018, the disclosure of each of which are hereby incorporated by reference in their entirety.

### SEQUENCE LISTING

The instant application contains a Sequence Listing which has been submitted electronically in XML file format and is hereby incorporated by reference in its entirety. Said XML copy, created on Apr. 16, 2024, is named 102085_002008_SL.xml and is 478,745 bytes in size.

### TECHNICAL FIELD

The instant application is directed to human antibody molecules that immunospecifically bind to human CXCR2.

### BACKGROUND

Neutrophils are the most abundant leukocytes in the blood. They are important effector cells of innate immunity, with a primary role in the clearance of extracellular pathogens. However, if neutrophil recruitment to tissue is inadequately controlled, chronic infiltration and activation of neutrophils may result in the persistent release of inflammatory mediators and proteinases which cause overt tissue damage.

The migration and activation of neutrophils is moderated through the interaction of CXC chemokine receptor 1 (CXCR1) and CXC chemokine receptor 2 (CXCR2) on the plasma membrane of the neutrophil with ELR+CXC chemokines (CXCL1, 2, 3, 5, 6, 7, and 8). CXCR2 acts as a high affinity receptor for all ELR+CXC chemokines and plays a key role in the mobilization and recruitment of neutrophils and monocytes from the blood to tissue. The chemokines CXCL6, 7, and 8 also interact with CXCR1, which modulates respiratory burst activity and protease release from neutrophils, and which is critical for immunity to bacteria and fungi.

Increased neutrophil counts in sputum have been associated with phenotypes associated with increased asthma severity, corticosteroid insensitivity, and chronic airflow obstruction. Airway neutrophilia is increased during acute asthma exacerbations. Airway neutrophilia is also a feature of all clinical phenotypes of chronic obstructive pulmonary disease (COPD) including COPD with a predominance of emphysema, COPD with frequent exacerbations, and COPD with evidence of high eosinophil activity. The degree of airway neutrophilia also correlates with severity of disease and rate of physiological decline. Neutrophil proteinases, especially neutrophil elastase, are implicated in all pathological features of COPD. Proteinases released by neutrophils are also associated with the development of emphysema, contribute to destruction of the extracellular matrix, and are associated with mucus hypersecretion. These associations suggest that neutrophil infiltration into the airways may have a crucial role in the pathophysiological processes underlying severe asthma and COPD.

CXCL1, CXCL5, and CXCL8 are CXCR2-binding chemokines which are implicated in neutrophil recruitment. CXCL1, CXCL5, and CXCL8 are upregulated in chronic airway inflammation and elevated in sputum or in bronchial biopsy material from subjects with severe neutrophilic asthma or COPD. Antagonizing the chemokine activation of CXCR2 offers a potential therapeutic strategy by reducing neutrophil recruitment into tissues and neutrophil mediated pathologies associated with these inflammatory diseases.

Chemokine receptors, however, have proven to be difficult targets to antagonise selectively. Despite difficulties in developing compounds with a desirable target specificity and antagonist activity, several small-molecule CXCR2 antagonists have proven effective in animal models of inflammation. Human clinical trials of small molecule CXCR2 antagonists in subjects with neutrophilic asthma or COPD have not demonstrated broad efficacy, even though studies of inhaled ozone- and lipopolysaccharide-induced sputum neutrophilia in otherwise normal human subjects demonstrated marked efficacy. Only a modest improvement in baseline lung function (FEV₁) was observed in COPD patients who were current smokers when compared with ex-smokers. To date, all published clinical trials have used small molecule CXCR2 antagonists. The most studied is danirixin (GSK1325756), a reversible and selective CXCR2 antagonist (IC₅₀ for CXCL8 binding=12.5 nM), which has also shown to block CD11b upregulation on neutrophils. (See, e.g. Miller et al., BMC Pharmacol Toxicol 2015; 16: 18). Danirixin failed to meet primary end points in a Phase IIb trial for COPD. Other CXCR2 selective molecules include SB-566933 (Lazaar et al., Br. J. Clin. Pharmacol. 2011; 72: 282-293) and AZD5069, which is CXCR2 selective (>150-fold less potent at CXCR1 and CCR2b receptors) and has no effect on C5a, LTB4 or fMLP induced CD11b expression) (Nicolls et al., J. Pharmacol Exp. Ther. 2015; 353: 340-350). Molecules which inhibit both CXCR2 and CXCR1 include navarixin (SCH 527123, MK-7123; Todd et al., Pulm Pharmacol Ther. 2016 December; 41: 34-39), and ladarixin (DF2156A; Hirose et al., J Genet Syndr Gene Ther 2013, S3). These molecules are being investigated for multiple indications, including COPD, asthma and other inflammatory lung conditions, cancer, and more.

In some studies, the use of small molecule CXCR2 antagonists resulted in a marked undesirable reduction in circulating neutrophils (neutropenia), which potentially limits the tolerable dose of such agents. Neutropenia may be a result of the antagonist not being completely specific to CXCR2, and/or if the antagonist was active across all CXCR2-binding ligands. CXCL8 and related CXC chemokines, for example, have a significant role in mobilizing mature granulocytes into peripheral blood, and consequently strong antagonism of these ligands on CXCR2 may prevent the normal migration of neutrophils to the blood. Conversely, preferential antagonism of the downstream pathway involving calcium flux signalling following ligand binding to CXCR2 may antagonize undesirable levels of migration of neutrophils into the lungs, whilst retaining the desirable ability of neutrophils to be mobilized into the blood.

### SUMMARY

Disclosed herein are human antibody molecules that immunospecifically bind to human CXCR2. The disclosed human antibody molecules are more selective antagonists of CXCR2 than currently described small molecule CXCR2 antagonists, more potent antagonists of CXCL1 and CXCL5 activation of CXCR2 than currently described antibody antagonists of CXCR2, and antagonize the recruitment of neutrophils into tissues without strongly depleting circulating neutrophil numbers. The human antibody molecules comprise the heavy chain CDR1, CDR2, and CDR3 of SEQ ID NO: 167 and the light chain CDR1, CDR2, and CDR3 of SEQ ID NO: 168 or the heavy chain CDR1, CDR2, and CDR3 of SEQ ID NO: 226 and the light chain CDR1, CDR2, and CDR3 of SEQ ID NO: 227, and inhibit activation of human CXCR2 by human CXCL1 or human CXCL5. In certain embodiments the disclosed human antibody molecules are able to inhibit activation of CXCR2 by CXCL1 or CXCL5 in a subject without inducing severe, sustained neutropenia.

Pharmaceutical compositions comprising the human antibody molecules are also provided.

Also disclosed are nucleic acid molecules encoding the human antibody

molecules, vectors comprising the nucleic acid molecules, and cells transformed to express the human antibody molecules.

Methods of preventing or treating neutrophilia in a peripheral tissue of a subject, such as airway neutrophilia, are also disclosed herein. Also disclosed herein are methods of reducing monocytes in a peripheral tissue of a subject. Also disclosed are methods of reducing eosinophilia in a peripheral tissue of a subject.

Also disclosed herein are methods of reducing acute airway inflammation, methods of preventing or reducing chronic airway inflammation for example in bronchiectasis, methods of reducing tumor burden, methods of arresting or slowing the growth of a cancer, methods of reducing chronic pain, methods of preventing or reducing neuroinflammation such as in multiple sclerosis, methods of reducing inflammation in the liver, methods of reducing inflammation in the pancreas or methods of reducing the symptoms of type I diabetes. The methods comprise administering to the subject a therapeutically- or prophylactically-effective amount of any of the disclosed human antibody molecules or any of the disclosed pharmaceutical compositions to treat or prevent the disclosed condition in the subject.

Also provided are the disclosed human antibody molecules or pharmaceutical compositions for use in the prevention or treatment of airway neutrophilia or acute lung inflammation. Also provided is the use of the human antibodies molecules or pharmaceutical compositions in the manufacture of a medicament for the prevention or treatment of airway neutrophilia or acute lung inflammation.

### BRIEF DESCRIPTION OF THE DRAWINGS

The summary, as well as the following detailed description, is further understood when read in conjunction with the appended drawings. For the purpose of illustrating the disclosed human antibody molecules, methods, and uses, there are shown in the drawings exemplary embodiments of the human antibody molecules, methods, and uses; however, the human antibody molecules, methods, and uses are not limited to the specific embodiments disclosed. In the drawings:

FIG. 1A and FIG. 1B illustrate the results of an exemplary dose response inhibition study of CXCL8-induced activation of CXCR2 by exemplary disclosed anti-CXCR2 antibodies as measured in the Tango™ cell based assay.

FIG. 2 illustrates a dot plot showing binding activity of exemplary disclosed anti-CXCR2 antibodies to native human CXCR2 expressed by human neutrophils and native cynomolgus CXCR2 expressed by cynomolgus neutrophils. Antibody binding activity was quantified as average mean fluorescence intensity (MFI) values obtained from 4 to 8 independent samples.

FIG. 3 illustrates the results of an exemplary dose response study of the inhibition of CXCL1-mediated activation of CXCR2 by selected anti-CXCR2 antibodies, as measured in the Tango™ cell based assay.

FIG. 4A and FIG. 4B illustrate an amino acid sequence alignment of exemplary BKO-4A8 variants with similar potency to parental BKO-4A8, and provides a consensus sequence. FIG. 4A=variable heavy chain sequences (BKO-4A8 SEQ ID NO:17; consensus sequence SEQ ID NO:167); FIG. 4B=variable light chain sequences (BKO-4A8 SEQ ID NO:18; consensus sequence SEQ ID NO: 168). The positioning of the CDRs within these sequences is according to Kabat. Accordingly, the 53rd amino acid residue in the alignment in FIG. 4A is numbered 52a according to Kabat (although the G52aD variant has a G to D change at the 53rd residue, the residue is named 52a). Similarly, in FIG. 4B, the 96th amino acid residue is named 95a. FIG. 4A discloses SEQ ID NOs: 17, 53-75, and 167 and the CDR1, CDR2, and CDR3 sequences of SEQ ID NOs: 169-171, respectively, in order of appearance. FIG. 4B discloses SEQ ID NOs: 18, 76-97, and 168 and the CDR1, CDR2, and CDR3 sequences of SEQ ID NOs: 172-174, respectively, in order of appearance.

FIG. 5A and FIG. 5B illustrate an amino acid sequence alignment of exemplary combinatorial BKO-4A8 variants (abbreviated “Var”) with parental BKO-4A8, and provides a consensus sequence based on these variants. FIG. 5A=variable heavy chain sequences (BKO-4A8 SEQ ID NO:17; consensus sequence SEQ ID NO:226); FIG. 5B=variable light chain sequences (BKO-4A8 SEQ ID NO:18; consensus sequence SEQ ID NO:227). FIG. 5A discloses SEQ ID NOs: 17, 98-108, 163-165, and 226 and the CDR1, CDR2, and CDR3 sequences of SEQ ID NOs: 228-230, respectively, in order of appearance. FIG. 5B discloses SEQ ID NOs: 18, 109-110, and 227 and the CDR1, CDR2, and CDR3 sequences of SEQ ID NOs: 201 and 231-232, respectively, in order of appearance.

FIG. 6A and FIG. 6B illustrate the results of a dose response inhibition study of (FIG. 6A) CXCL1- and (FIG. 6B) CXCL8-mediated activation of CXCR2 by select anti-CXCR2 antibodies BKO-4A8 and the optimized antibodies BKO-4A8-101c, BKO-4A8-103c, BKO-4A8-104c and BKO-4A8-105c as measured in the Tango™ cell based assay.

FIG. 7 illustrates the results of a dose response inhibition study of ELR+CXC chemokine-mediated activation of CXCR2 by the disclosed anti-CXCR2 antibody BKO-4A8-101c as measured in the Tango™ cell based assay.

FIG. 8 illustrates the results of a dose response inhibition study of CXCL1-, CXCL5- and CXCL8-mediated activation of CXCR2 by the disclosed anti-CXCR2 antibody BKO-4A8-101c as measured in a calcium flux assay.

FIG. 9 illustrates human CXCR2 binding activity of the disclosed anti-CXCR2 antibody BKO-4A8 formatted onto different human IgG constant regions, as determined by flow cytometry analysis.

FIG. 10 illustrates the results of a dose response inhibition study of CXCL8-mediated activation of the disclosed anti-CXCR2 antibody BKO-4A8 formatted onto different human IgG constant regions, as measured in the Tango™ cell based assay.

FIG. 11 illustrates results from binding studies of the anti-CXCR2 antibody BKO-4A8-101c with a variety of human CXCR family members. The results demonstrated that BKO-4A8-101c only bound to CXCR2 amongst the human CXCR family members.

FIG. 12A and FIG. 12B illustrate an exemplary binding profile of BKO-4A8-101c (shaded histogram) to phenotypically defined human peripheral blood hematopoietic cells assessed by flow cytometry (N=8), incorporating isotype controls (human-IgG unshaded histogram). Expression was high on neutrophils (FIG. 12A), while monocytes (FIG. 12B) expressed intermediate levels of CXCR2.

FIG. 13A, FIG. 13B, and FIG. 13C illustrate the results of a subcutaneous sensitization and intranasal challenge (on Day 14) using House Dust Mite (HDM) antigen, which induced features of acute allergic (asthma-like) inflammation in the lungs of human-CXCR2 transgenic mice. Specifically, following challenge mice demonstrated a moderate to marked multifocal pulmonary inflammation with eosinophils, and mild to moderate bronchiolar goblet cell hyperplasia compared to control (naïve) mice that had little to no inflammation. Treatment with BKO-4A8-mIgG resulted in a reduction in the severity of pathology including significant reductions in lung neutrophil (FIG. 13A) and lung eosinophil counts (FIG. 13B) and mucus density score (FIG. 13C) compared to vehicle.

FIG. 14A and FIG. 14B illustrate results of anti-CXCR2 activity of the antibody BKO-4A8-101c in a cynomolgus monkey model of acute lung inflammation. Aerosol inhalation of lipopolysaccharide (LPS) (on Day 0) successfully induced features of acute neutrophilic inflammation in the lungs of cynomolgus monkeys. Treatment with anti-CXCR2 antibody BKO-4A8-101c (1 mg/kg) 1 hour prior to challenge with LPS on day 0 resulted in a significant reduction in bronco-alveolar lavage neutrophil counts 24 hours after LPS challenge. (FIG. 14A) Peripheral blood neutrophil counts were not impacted by three repeat administrations of BKO-4A8-101c given at two weekly intervals on Day 0, 14 and 28. Group median and range shown, N=4. (FIG. 14B)

FIG. 15A, FIG. 15B, and FIG. 15C show selective inhibition of chemokine-induced upregulation of CD11b on enriched human neutrophils. Anti-CXCR2 antibody significantly inhibited the response to CXCL1 (p=0.0002) (FIG. 15A) and CXCL5 (p=0.0001) (FIG. 15B). Anti-CXCR2 antibody was significantly more inhibitory than the small-molecule antagonist 5 in the same assays (p<0.0058) (FIG. 15A-B). CXCL8-mediated CD11b upregulation was reduced by a CXCR1 antagonist (data not shown), but not by anti-CXCR2 antibody or antagonist 5 (FIG. 15C).

### DETAILED DESCRIPTION OF ILLUSTRATIVE EMBODIMENTS

The disclosed human antibody molecules, methods, and uses may be understood more readily by reference to the following detailed description taken in connection with the accompanying figures, which form a part of this disclosure. It is to be understood that the disclosed human antibody molecules, methods, and uses are not limited to the specific human antibody molecules, methods, and uses described and/or shown herein, and that the terminology used herein is for the purpose of describing particular embodiments by way of example only and is not intended to be limiting of the claimed human antibody molecules, methods, and uses.

Unless specifically stated otherwise, any description as to a possible mechanism or mode of action or reason for improvement is meant to be illustrative only, and the disclosed human antibody molecules, methods, and uses are not to be constrained by the correctness or incorrectness of any such suggested mechanism or mode of action or reason for improvement.

Throughout this text, the descriptions refer to human antibody molecules and methods of using said human antibody molecules. Where the disclosure describes or claims a feature or embodiment associated with a human antibody molecule, such a feature or embodiment is equally applicable to the methods of using said human antibody molecule. Likewise, where the disclosure describes or claims a feature or embodiment associated with a method of using a human antibody molecule, such a feature or embodiment is equally applicable to the human antibody molecule.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value. It is not intended that the scope of the invention be limited to the specific values recited when defining a range. All ranges are inclusive and combinable.

It is to be appreciated that certain features of the disclosed human antibody molecules, methods, and uses which are, for clarity, described herein in the context of separate embodiments, may also be provided in combination in a single embodiment. Conversely, various features of the disclosed human antibody molecules, methods, and uses that are, for brevity, described in the context of a single embodiment, may also be provided separately or in any subcombination.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used herein, the singular forms “a,” “an,” and “the” include the plural.

The term “about” when used in reference to numerical ranges, cutoffs, or specific values is used to indicate that the recited values may vary by up to as much as 10% from the listed value. As many of the numerical values used herein are experimentally determined, it should be understood by those skilled in the art that such determinations can, and often times will, vary among different experiments. The values used herein should not be considered unduly limiting by virtue of this inherent variation. Thus, the term “about” is used to encompass variations of ±10% or less, variations of ±5% or less, variations of ±1% or less, variations of ±0.5% or less, or variations of ±0.1% or less from the specified value. When values are expressed by use of the antecedent “about” it will be understood that the particular value forms another embodiment.

Reference to a particular numerical value includes at least that particular value, unless the context clearly dictates otherwise.

The term “comprising” is intended to include examples encompassed by the terms “consisting essentially of” and “consisting of”; similarly, the term “consisting essentially of” is intended to include examples encompassed by the term “consisting of.”

As used herein, “wherein the antibody molecule inhibits CXCL1-induced activation of CXCR2 or CXCL5-induced activation of CXCR2” and like phrases refers to the ability of the disclosed human antibody molecules to reduce CXCL1-induced or CXCL5-induced CXCR2 activation as determined in a β-arrestin recruitment in a Tango™ cell based assay by about 80%, about 85%, about 90%, about 92%, about 95%, about 97%, or about 100% compared to the level of CXCL1- and/or CXCL5-induced CXCR2 activation in the absence of the disclosed human antibody molecules and with an IC₅₀ of from 0.08 to 0.5 nM at a concentration of from 1.5-3.4 nM for CXCL1 and from 47.7 to 150 nM for CXCL5.

As used herein, “treating” and like terms refers to at least one of reducing the severity and/or frequency of symptoms, eliminating symptoms, ameliorating or eliminating the underlying cause of the symptoms, reducing the frequency or likelihood of symptoms and/or their underlying cause, and/or improving or remediating damage caused, directly or indirectly, by the described conditions or disorders. Treating may also include prolonging survival as compared to the expected survival of a subject not receiving the disclosed human antibody molecules or pharmaceutical compositions comprising the same.

As used herein, “preventing” and like terms refers to prophylactic or maintenance measures. Subjects for receipt of such prophylactic or maintenance measures include those who are at risk of having the described conditions or disorders due to, for example, genetic predisposition or environmental factors, or those who were previously treated for having the described conditions or disorders and are receiving therapeutically effective doses of the disclosed human antibody molecules or pharmaceutical compositions as a maintenance medication (e.g. to maintain low levels of lung neutrophils).

As used herein, “administering to the subject” and similar terms indicate a procedure by which the disclosed human antibody molecules or pharmaceutical compositions comprising the same are injected into/provided to a patient such that target cells, tissues, or segments of the body of the subject are contacted with the disclosed human antibody molecules.

The phrase “therapeutically effective amount” refers to an amount of the disclosed human antibody molecules or pharmaceutical compositions comprising the same, as described herein, effective to achieve a particular biological or therapeutic or prophylactic result such as, but not limited to, biological or therapeutic results disclosed, described, or exemplified herein. The therapeutically effective amount may vary according to factors such as the disease state, age, sex, and weight of the individual, and the ability of the composition to cause a desired response in a subject. Exemplary indicators of a therapeutically effect amount include, for example, improved well-being of the subject, a reduction in neutrophilia in one or more peripheral tissues, such as a reduction in airway neutrophilia, a reduction in the numbers of monocytes in one or more peripheral tissues, a reduction of acute airway inflammation, a reduction of chronic airway inflammation for example in bronchiectasis, a reduction of a tumor burden, arrested or slowed growth of a cancer, a reduction in chronic pain, a reduction in neuroinflammation such as in multiple sclerosis, a reduction in inflammation in the liver, a reduction of inflammation in the pancreas, or a decrease in the symptoms of type I diabetes.

As used herein, “pharmaceutically acceptable carrier” or “pharmaceutical acceptable excipient” includes any material which, when combined with an active ingredient (such as the disclosed human antibody molecules), allows the ingredient to retain biological activity and is non-reactive with the subject's immune system. Examples include, but are not limited to, any of the standard pharmaceutical carriers such as a phosphate buffered saline solution, water, and various types of wetting agents (such as polysorbate 20, polysorbate 80, and salts of tris(hydroxymethyl)aminomethane (“Tris”), such as the hydrochloride, acetate, maleate and lactate salts. Also may be added as stabilizing agents are amino acids (such as histidine, glutamine, glutamate, glycine, arginine), sugars (such as sucrose, glucose, trehalose), chelators (e.g. ETDA), and antioxidants (e.g. reduced cysteine). Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000). In particular embodiments the pharmaceutical composition is a composition for parenteral delivery.

The term “subject” as used herein is intended to mean monkeys, such as cynomolgus macaques, and humans, and most preferably humans. “Subject” and “patient” are used interchangeably herein.

The term “antibody” and like terms is meant in a broad sense and includes immunoglobulin molecules including, monoclonal antibodies, antibody fragments, bispecific or multispecific antibodies, dimeric, tetrameric or multimeric antibodies, and single chain antibodies. Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

“Antibody fragment” refers to a portion of an immunoglobulin molecule that retains the specific antigen binding properties of the parental full length antibody (i.e. antigen-binding fragment thereof). Exemplary antibody fragments comprise heavy chain complementarity determining regions (HCDR) 1, 2, and 3 and light chain complementarity determining regions (LCDR) 1, 2, and 3. Other exemplary antibody fragments comprise a heavy chain variable region (VH) and a light chain variable region (VL). Antibody fragments include without limitation: an Fab fragment, a monovalent fragment consisting of the VL, VH, constant light (CL), and constant heavy 1 (CH1) domains; an F(ab)₂ fragment, a bivalent fragment comprising two Fab fragments linked by a disulfide bridge at the hinge region; and an Fv fragment consisting of the VL and VH domains of a single arm of an antibody. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody; described for example in Int'l Pat. Pub. Nos. WO1998/044001, WO1988/001649, WO1994/013804, and WO1992/001047. These antibody fragments are obtained using techniques well known to those of skill in the art, and the fragments are screened for utility in the same manner as are full length antibodies.

Each antibody heavy chain or light chain variable region consists of four “framework” regions (FRs) which alternate with three “Complementarity Determining Regions” (CDRs), in the sequence FR1, CDR1, FR2, CDR2, FR3, CDR3, FR4 (from amino to carboxy termini). The three CDRs in the VH are identified as HCDR1, HCDR2, HCDR3, and the three CDRs in the VL are identified as LCDR1, LCDR2, LCDR3 respectively. The location and size of the CDRs are defined based on rules which identify regions of sequence variability within the immunoglobulin variable regions (Wu and Kabat J Exp Med 132:211-50, 1970; Kabat et al. Sequences of Proteins of Immunological Interest, 5th Ed. Public Health Service, National Institutes of Health, Bethesda, Md., 1991). Amino acid residues within a variable region may be numbered according the scheme of Kabat (ibid.) “Frameworks” or “framework regions” are the remaining sequences of a variable region other than those defined to be CDRs.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin if the variable regions of the antibody are obtained from a system that uses human germline immunoglobulin or rearranged human immunoglobulin genes. Such systems include human immunoglobulin gene libraries displayed on phage, and transgenic non-human animals such as mice or rats carrying human immunoglobulin loci. “Human antibody” may contain amino acid differences when compared to the human germline or rearranged immunoglobulin sequences due to, for example, naturally occurring somatic mutations or intentional introduction of substitutions in the framework or antigen binding sites. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene. In some cases, a “human antibody” may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, as described in, for example, Shi et al., J Mol Biol 397:385-96, 2010 and Int'l Pat. Pub. No. WO2009/085462.

Human antibodies, while derived from human immunoglobulin sequences, may be generated using systems such as phage display incorporating synthetic CDRs and/or synthetic frameworks, and/or can be subjected to in vitro mutagenesis to improve antibody properties in the variable regions or the constant regions or both, resulting in antibodies that do not naturally exist within the human antibody germline repertoire in vivo.

“Monoclonal antibody” refers to a population of antibody molecules of a substantially single molecular composition. A monoclonal antibody composition displays a single binding specificity and affinity for a particular epitope, or in a case of a bispecific monoclonal antibody, a dual binding specificity to two distinct epitopes. Monoclonal antibody therefore refers to an antibody population with single amino acid composition in each heavy and each light chain, except for possible well known alterations such as removal of C-terminal lysine from the antibody heavy chain, and processing variations in which there is incomplete cleavage of the N-terminal leader sequence that is produced in the cell and ordinarily cleaved upon secretion. For example, U.S. Pat. No. 8,241,630 describes a commercial antibody in which 5-15% of the antibody population retain the leader sequence. Monoclonal antibodies may have heterogeneous glycosylation within the antibody population. Monoclonal antibody may be monospecific or multispecific, or monovalent, bivalent or multivalent. A bispecific antibody is included in the term monoclonal antibody.

“Epitope” refers to a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” refers to a polypeptide or a polynucleotide that differs from a reference polypeptide or a reference polynucleotide by one or more modifications for example, substitutions, insertions, or deletions.

The phrase “immunospecifically binds” refers to the ability of the disclosed human antibody molecules to preferentially bind to its target (CXCR2 in the case of “anti-CXCR2 antibody) without preferentially binding other molecules of the CXCR family in a sample containing a mixed population of molecules. Human antibody molecules that immunospecifically bind CXCR2 are substantially free of other antibodies having different antigenic specificities (e.g., an anti-CXCR2 antibody is substantially free of antibodies that specifically bind antigens other than CXCR2). Antibody molecules that immunospecifically bind human CXCR2, however, can have cross-reactivity to other antigens, such as orthologs of human CXCR2, including Macaca fascicularis (cynomolgus monkey) CXCR2. The antibody molecules disclosed herein are able to immunospecifically bind both naturally produced human CXCR2 and to human CXCR2 which is recombinantly produced in mammalian or prokaryotic cells.

As used herein, “severe, sustained neutropenia” refers to an absolute peripheral blood neutrophil count (ANC) less than 0.4×10⁹ cells/L for greater than 2 weeks. Severe, sustained neutropenia can be graded as follows:
Grade 1 indicates a mild event (0.8-1.0×10⁹ cells/L)
Grade 2 indicates a moderate event (0.6-0.8×10⁹ cells/L)
Grade 3 indicates a severe event (0.4-0.6×10⁹ cells/L)
Grade 4 indicates a potentially life threatening event (less than 0.4×10⁹ cells/L)

### (See Division of AIDS (DAIDS) National Institute of Allergy and Infectious Diseases National Institutes of Health US Department of Health and Human Services Table for Grading the Severity of Adult and Pediatric Adverse Events, Version 2.0 November 2014).

The following abbreviations are used herein: variable heavy chain (VH); variable light chain (VL); complementarity-determining region (CDR); heavy chain CDR (HCDR); light chain CDR (LCDR); CXC chemokine receptor 2 (CXCR2); and chemokine ligand 1, 2, 3, 5, 6, 7, and 8 (CXCL1, 2, 3, 5, 6, 7, and 8).

The disclosed antibody molecules can comprise one or more substitutions, deletions, or insertions, in the framework and/or constant regions. In some embodiments, an IgG4 antibody molecule can comprise a S228P substitution. S228 (residue numbering according to EU index) is located in the hinge region of the IgG4 antibody molecule. Substitution of the serine (“S”) to a proline (“P”) serves to stabilize the hinge of the IgG4 and prevent Fab arm exchange in vitro and in vivo. In some embodiments, the antibody molecules can comprise one or more modifications which increase the in vivo half-life of the antibody molecules. For instance in certain embodiments the antibody can comprise a M252Y substitution, a S254T substitution, and a T256E substitution (collectively referred to as the “YTE” substitution). M252, S254, and T256 (residue numbering according to EU index) are located in in the CH2 domain of the heavy chain. Substitution of these residues to tyrosine (“Y”), threonine (“T”), and glutamate (“E”), respectively, protects the antibody molecules from lysosomal degradation, thereby enhancing the serum half-life of the antibody molecules. In some embodiments, the antibody molecules can comprise a deletion of the heavy chain C-terminal lysine residue. Deletion of the heavy chain C-terminal lysine residue reduces heterogeneity of the antibody molecules when produced by mammalian cells. In some embodiments, the antibody molecules can comprise a combination of substitutions, deletions, or insertions. For example, in some aspects, the disclosed antibody molecules can comprise a S228P substitution and a deletion of a heavy chain C-terminal lysine residue. Antibody constant regions of different classes are known to be involved in modulating antibody effector functions such as antibody-dependent cellular cytotoxicity (ADCC), complement-dependent cytotoxicity (CDC), and antibody dependent phagocytosis (ADP). In some embodiments the disclosed antibody molecules may comprise one or more substitutions, deletions, or insertions in the constant regions which modulate one or more antibody effector functions, such as reducing or ablating one or more effector functions. Other alterations that affect antibody effector functions and circulation half-life are known. See, e.g. Saunders KO “Conceptual Approaches to Modulating Antibody Effector Functions and Circulation Half-Life” Front. Immunol. (2019) 10:1296.

### Human Antibody Molecules

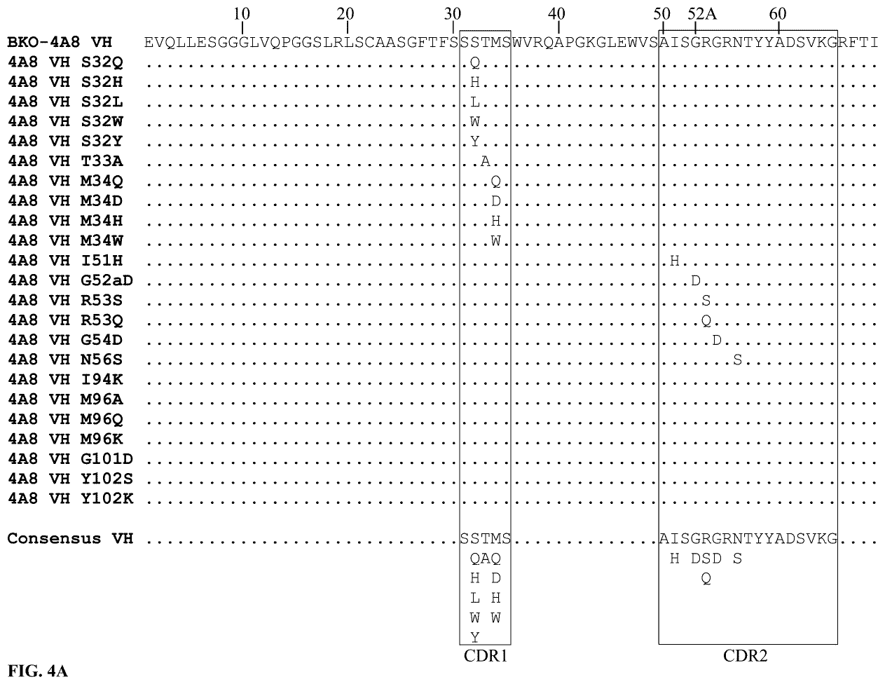

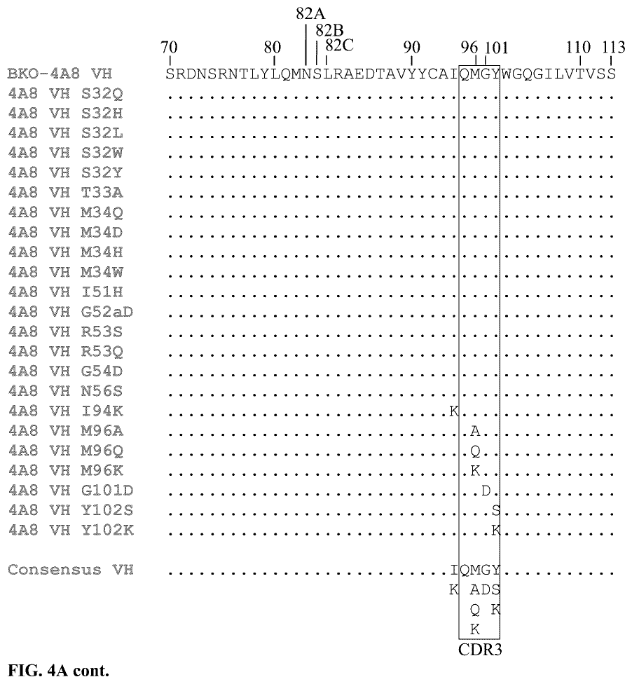

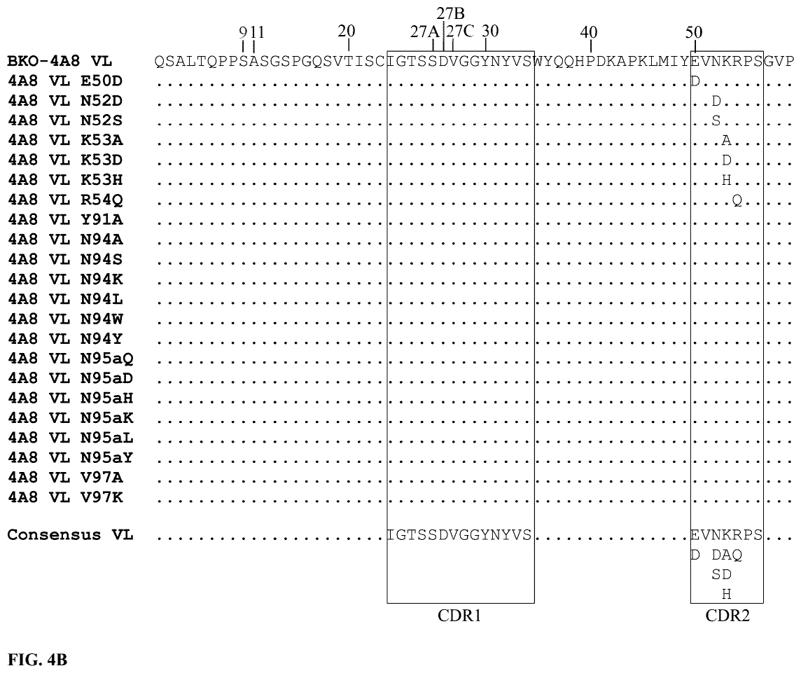

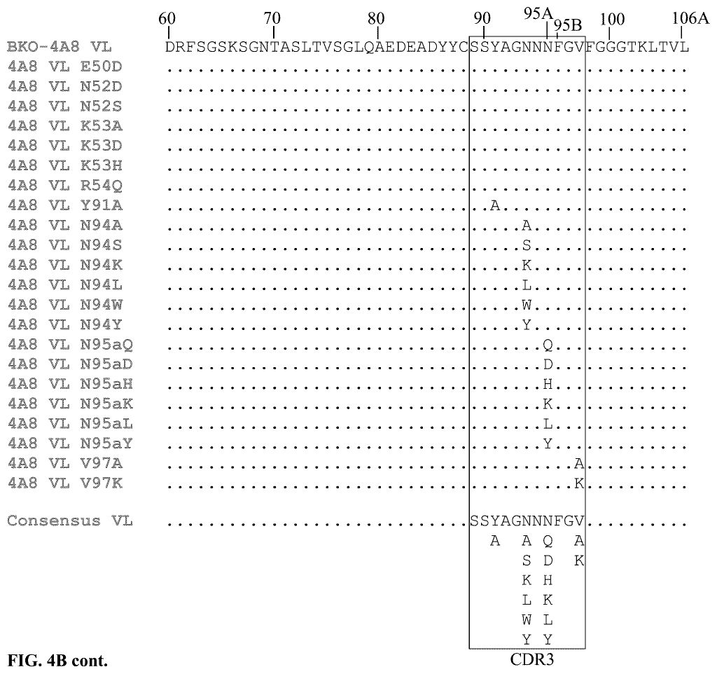

Disclosed herein are human antibody molecules that immunospecifically bind to human CXCR2. The human antibody molecules can comprise the heavy chain CDR1, CDR2, and CDR3 of SEQ ID NO: 167 and the light chain CDR1, CDR2, and CDR3 of SEQ ID NO: 168 and inhibit activation of CXCR2 by CXCL1 or CXCL5. As provided in Table 19 and FIGS. 4A and 4B, SEQ ID NOS: 167 and 168 represent a consensus heavy chain variable region and light chain variable region, respectively (SEQ ID NO: 167=“consensus VH” and SEQ ID NO: 168=“consensus VL”), of the disclosed human antibody molecules. Consensus CDR sequences are provided as SEQ ID NOs: 169, 170, 171, 172, 173, and 174. The numbering in the names of the disclosed CDR sequences, unless otherwise noted, is according to Kabat. In some embodiments, the human antibody molecules can comprise:
the heavy chain CDR1 comprising the amino acid sequence of SX₁X₂X₃S wherein: X₁ is S, Q, H, L, W, or Y; X₂ is T or A; and X₃ is M, Q, D, H, or W as provided in SEQ ID NO: 169;
the heavy chain CDR2 comprising the amino acid sequence of AX₄SX₅X₆X₇RX₈TYYADSVKG wherein: X₄ is I or H; X₅ is G or D; X₆ is R, S, or Q; X₇ is G or D; and X₈ is N or S as provided in SEQ ID NO: 170;
the heavy chain CDR3 comprising the amino acid sequence of QX₁₀X₁₁X₁₂ wherein: X₁₀ is M, A, Q, or K; X₁₁ is G or D; and X₁₂ is Y, S, or K as provided in SEQ ID NO: 171;
the light chain CDR1 comprising the amino acid sequence of IGTSSDVGGYNYVS as provided in SEQ ID NO: 172;
the light chain CDR2 comprising the amino acid sequence of X₁₃VX₁₄X₁₅X₁₆PS wherein: X₁₃ is E or D; X₁₄ is N, D, or S; X₁₅ is K, A, D, or H; and X₁₆ is R or Q as provided in SEQ ID NO: 173; and
the light chain CDR3 comprising the amino acid sequence of SSX₁₇AGX₁₈NX₁₉FGX₂₀ wherein: X₁₇ is Y or A; X₁₈ is N, A, S, K, L, W, or Y; X₁₉ is N, Q, D, H, K, L, or Y; and X₂₀ is V, A, or K as provided in SEQ ID NO: 174.

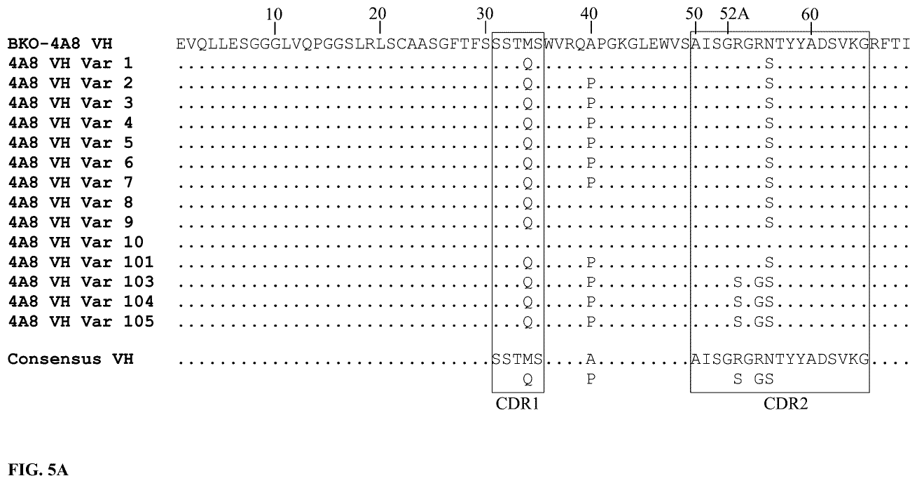

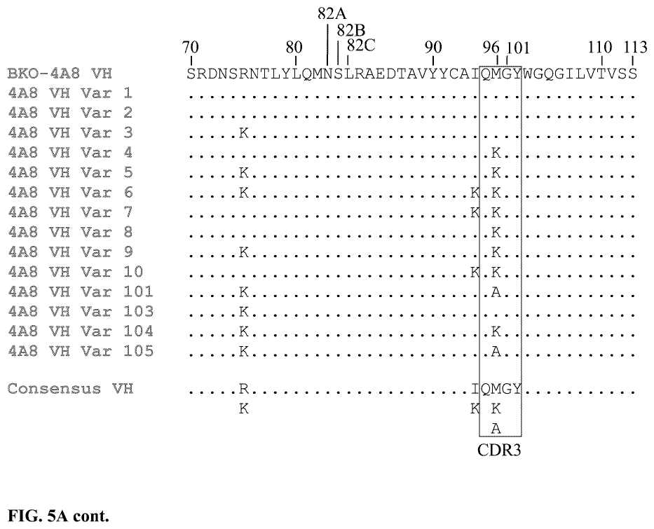

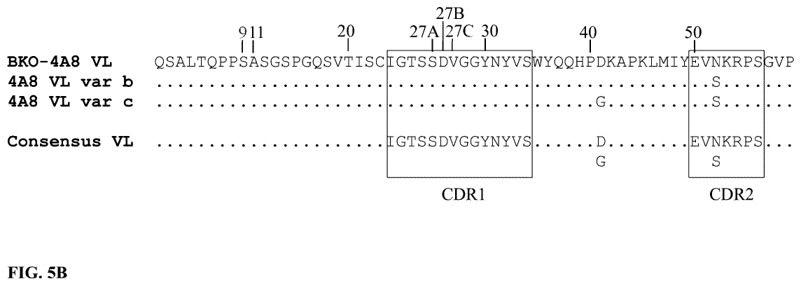

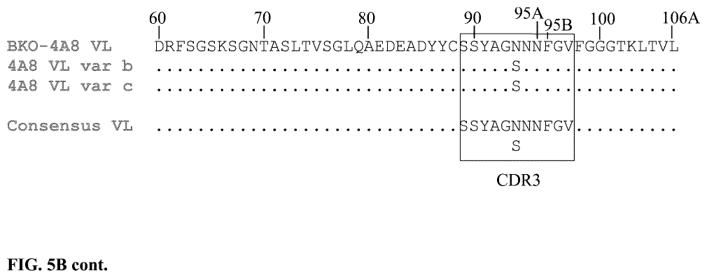

The human antibody molecules can comprise the heavy chain CDR1, CDR2, and CDR3 of SEQ ID NO: 226 and the light chain CDR1, CDR2, and CDR3 of SEQ ID NO: 227. As provided in Table 19 and FIGS. 5A and 5B, SEQ ID NO: 226 and 227 represent a consensus heavy chain variable region and light chain variable region, respectively (SEQ ID NO: 226=“consensus VH” and SEQ ID NO: 227=“consensus VL”), of the disclosed human antibody molecules. Consensus CDR sequences are provided as SEQ ID NOs: 228, 229, 230, 201, 231, and 232. In some embodiments, the human antibody molecules can comprise:
the heavy chain CDR1 comprising the amino acid sequence of SSTX₂₁S wherein X₂₁ is M or Q as provided in SEQ ID NO: 228;
the heavy chain CDR2 comprising the amino acid sequence of AISGX₂₃GX₂₄X₂₅TYYADSVKG wherein: X₂₃ is R or S; X₂₄ is R or G; and X₂₅ is N or S as provided in SEQ ID NO: 229;
the heavy chain CDR3 comprising the amino acid sequence of QX₂₈GY wherein: X₂₈ is M, K, or A as provided in SEQ ID NO: 230;
the light chain CDR1 comprising the amino acid sequence of IGTSSDVGGYNYVS as provided in SEQ ID NO: 201;
the light chain CDR2 comprising the amino acid sequence of EVX₃₀KRPS wherein: X₃₀ is N or S as provided in SEQ ID NO: 231; and
the light chain CDR3 comprising the amino acid sequence of SSYAGX₃₁NNFGV wherein: X₃₁ is N or S as provided in SEQ ID NO: 232.

The disclosed human antibody molecules can comprise a combination of the heavy chain and light chain CDRs provided in Table 1. In some embodiments, for example, the human antibody molecule can comprise a heavy chain CDR1 comprising the amino acid sequence of any one of SEQ ID NOs: 175-185, a heavy chain CDR2 comprising the amino acid sequence of any one of SEQ ID NOs: 186-192, a heavy chain CDR3 comprising the amino acid sequence of any one of SEQ ID NOs: 194-200, a light chain CDR1 comprising the amino acid sequence of SEQ ID NO: 201, a light chain CDR2 comprising the amino acid sequence of any one of SEQ ID NOs: 202-209, and a light chain CDR3 comprising the amino acid sequence of any one of SEQ ID NOs: 210-225.

TABLE 1
Heavy chain and light chain CDR sequences

| | | | |
|---|---|---|---|
| Antibody chain with | | | |
| substitution(s) identified | | | |
| by Kabat position | | | |
| SEQ ID NO: | CDR1 | CDR2 | CDR3 |

HC Variable Regions

| | | | |
|---|---|---|---|
| 4A8 VH S32Q | SEQ ID NO: 176 | SEQ ID NO: 186 | SEQ ID NO: 194 |
| SEQ ID NO: 53 | | | |
| 4A8 VH S32H | SEQ ID NO: 177 | SEQ ID NO: 186 | SEQ ID NO: 194 |
| SEQ ID NO: 54 | | | |
| 4A8 VH S32L | SEQ ID NO: 178 | SEQ ID NO: 186 | SEQ ID NO: 194 |
| SEQ ID NO: 55 | | | |
| 4A8 VH S32W | SEQ ID NO: 179 | SEQ ID NO: 186 | SEQ ID NO: 194 |
| SEQ ID NO: 56 | | | |
| 4A8 VH S32Y | SEQ ID NO: 180 | SEQ ID NO: 186 | SEQ ID NO: 194 |
| SEQ ID NO: 57 | | | |
| 4A8 VH T33A | SEQ ID NO: 181 | SEQ ID NO: 186 | SEQ ID NO: 194 |
| SEQ ID NO: 58 | | | |
| 4A8 VH M34Q | SEQ ID NO: 182 | SEQ ID NO: 186 | SEQ ID NO: 194 |
| SEQ ID NO: 59 | | | |
| 4A8 VH M34D | SEQ ID NO: 183 | SEQ ID NO: 186 | SEQ ID NO: 194 |
| SEQ ID NO: 60 | | | |
| 4A8 VH M34H | SEQ ID NO: 184 | SEQ ID NO: 186 | SEQ ID NO: 194 |
| SEQ ID NO: 61 | | | |
| 4A8 VH M34W | SEQ ID NO: 185 | SEQ ID NO: 186 | SEQ ID NO: 194 |
| SEQ ID NO: 62 | | | |
| 4A8 VH I51H | SEQ ID NO: 175 | SEQ ID NO: 187 | SEQ ID NO: 194 |
| SEQ ID NO: 63 | | | |
| 4A8 VH G52aD | SEQ ID NO: 175 | SEQ ID NO: 188 | SEQ ID NO: 194 |
| SEQ ID NO: 64 | | | |
| 4A8 VH R53S | SEQ ID NO: 175 | SEQ ID NO: 189 | SEQ ID NO: 194 |
| SEQ ID NO: 65 | | | |
| 4A8 VH R53Q | SEQ ID NO: 175 | SEQ ID NO: 190 | SEQ ID NO: 194 |
| SEQ ID NO: 66 | | | |
| 4A8 VH G54D | SEQ ID NO: 175 | SEQ ID NO: 191 | SEQ ID NO: 194 |
| SEQ ID NO: 67 | | | |
| 4A8 VH N56S | SEQ ID NO: 175 | SEQ ID NO: 192 | SEQ ID NO: 194 |
| SEQ ID NO: 68 | | | |
| 4A8 VH M96A | SEQ ID NO: 175 | SEQ ID NO: 186 | SEQ ID NO: 195 |
| SEQ ID NO: 70 | | | |
| 4A8 VH M96Q | SEQ ID NO: 175 | SEQ ID NO: 186 | SEQ ID NO: 196 |
| SEQ ID NO: 71 | | | |
| 4A8 VH M96K | SEQ ID NO: 175 | SEQ ID NO: 186 | SEQ ID NO: 197 |
| SEQ ID NO: 72 | | | |
| 4A8 VH G101D | SEQ ID NO: 175 | SEQ ID NO: 186 | SEQ ID NO: 198 |
| SEQ ID NO: 73 | | | |
| 4A8 VH Y102S | SEQ ID NO: 175 | SEQ ID NO: 186 | SEQ ID NO: 199 |
| SEQ ID NO: 74 | | | |
| 4A8 VH Y102K | SEQ ID NO: 175 | SEQ ID NO: 186 | SEQ ID NO: 200 |
| SEQ ID NO: 75 | | | |
| 4A8 VH M34Q, N56S | SEQ ID NO: 182 | SEQ ID NO: 192 | SEQ ID NO: 194 |
| (SEQ ID NO: 98) | | | |
| 4A8 VH M34Q, A40P, | SEQ ID NO: 182 | SEQ ID NO: 192 | SEQ ID NO: 194 |
| N56S | | | |
| (SEQ ID NO: 99) | | | |
| 4A8 VH M34Q, A40P, | SEQ ID NO: 182 | SEQ ID NO: 192 | SEQ ID NO: 194 |
| N56S, R75K | | | |
| (SEQ ID NO: 100) | | | |
| 4A8 VH M34Q, A40P, | SEQ ID NO: 182 | SEQ ID NO: 192 | SEQ ID NO: 197 |
| N56S, M96K | | | |
| (SEQ ID NO: 101) | | | |
| 4A8 VH M34Q, A40P, | SEQ ID NO: 182 | SEQ ID NO: 192 | SEQ ID NO: 197 |
| N56S, R75K, M96K | | | |
| (SEQ ID NO: 102) | | | |
| 4A8 VH M34Q, A40P, | SEQ ID NO: 182 | SEQ ID NO: 192 | SEQ ID NO: 197 |
| N56S, R75K, I94K, M96K | | | |
| (SEQ ID NO: 103) | | | |
| 4A8 VH M34Q, A40P, | SEQ ID NO: 182 | SEQ ID NO: 192 | SEQ ID NO: 197 |
| N56S, I94K, M96K | | | |
| (SEQ ID NO: 104) | | | |
| 4A8 VH M34Q, N56S, | SEQ ID NO: 182 | SEQ ID NO: 192 | SEQ ID NO: 197 |
| M96K | | | |
| (SEQ ID NO: 105) | | | |
| 4A8 VH M34Q, N56S, | SEQ ID NO: 182 | SEQ ID NO: 192 | SEQ ID NO: 197 |
| R75K, M96K | | | |
| (SEQ ID NO: 106) | | | |
| 4A8 VH I94K, M96K | SEQ ID NO: 175 | SEQ ID NO: 186 | SEQ ID NO: 197 |
| (SEQ ID NO: 107) | | | |
| 4A8 VH M34Q, A40P, | SEQ ID NO: 182 | SEQ ID NO: 192 | SEQ ID NO: 195 |
| N56S, R75K, M96A | | | |
| (SEQ ID NO: 108) | | | |

LC Variable Regions

| | | | |
|---|---|---|---|
| 4A8 VL E50D | SEQ ID NO: 201 | SEQ ID NO: 203 | SEQ ID NO: 210 |
| SEQ ID NO: 76 | | | |
| 4A8 VL N52D | SEQ ID NO: 201 | SEQ ID NO: 204 | SEQ ID NO: 210 |
| SEQ ID NO: 77 | | | |
| 4A8 VL N52S | SEQ ID NO: 201 | SEQ ID NO: 205 | SEQ ID NO: 210 |
| SEQ ID NO: 78 | | | |
| 4A8 VL K53A | SEQ ID NO: 201 | SEQ ID NO: 206 | SEQ ID NO: 210 |
| SEQ ID NO: 79 | | | |
| 4A8 VL K53D | SEQ ID NO: 201 | SEQ ID NO: 207 | SEQ ID NO: 210 |
| SEQ ID NO: 80 | | | |
| 4A8 VL K53H | SEQ ID NO: 201 | SEQ ID NO: 208 | SEQ ID NO: 210 |
| SEQ ID NO: 81 | | | |
| 4A8 VL R54Q | SEQ ID NO: 201 | SEQ ID NO: 209 | SEQ ID NO: 210 |
| SEQ ID NO: 82 | | | |
| 4A8 VL Y91A | SEQ ID NO: 201 | SEQ ID NO: 202 | SEQ ID NO: 211 |
| SEQ ID NO: 83 | | | |
| 4A8 VL N94A | SEQ ID NO: 201 | SEQ ID NO: 202 | SEQ ID NO: 212 |
| SEQ ID NO: 84 | | | |
| 4A8 VL N94S | SEQ ID NO: 201 | SEQ ID NO: 202 | SEQ ID NO: 213 |
| SEQ ID NO: 85 | | | |
| 4A8 VL N94K | SEQ ID NO: 201 | SEQ ID NO: 202 | SEQ ID NO: 214 |
| SEQ ID NO: 86 | | | |
| 4A8 VL N94L | SEQ ID NO: 201 | SEQ ID NO: 202 | SEQ ID NO: 215 |
| SEQ ID NO: 87 | | | |
| 4A8 VL N94W | SEQ ID NO: 201 | SEQ ID NO: 202 | SEQ ID NO: 216 |
| SEQ ID NO: 88 | | | |
| 4A8 VL N94Y | SEQ ID NO: 201 | SEQ ID NO: 202 | SEQ ID NO: 217 |
| SEQ ID NO: 89 | | | |
| 4A8 VL N95aQ | SEQ ID NO: 201 | SEQ ID NO: 202 | SEQ ID NO: 218 |
| SEQ ID NO: 90 | | | |
| 4A8 VL N95aD | SEQ ID NO: 201 | SEQ ID NO: 202 | SEQ ID NO: 219 |
| SEQ ID NO: 91 | | | |
| 4A8 VL N95aH | SEQ ID NO: 201 | SEQ ID NO: 202 | SEQ ID NO: 220 |
| SEQ ID NO: 92 | | | |
| 4A8 VL N95aK | SEQ ID NO: 201 | SEQ ID NO: 202 | SEQ ID NO: 221 |
| SEQ ID NO: 93 | | | |
| 4A8 VL N95aL | SEQ ID NO: 201 | SEQ ID NO: 202 | SEQ ID NO: 222 |
| SEQ ID NO: 94 | | | |
| 4A8 VL N95aY | SEQ ID NO: 201 | SEQ ID NO: 202 | SEQ ID NO: 223 |
| SEQ ID NO: 95 | | | |
| 4A8 VL V97A | SEQ ID NO: 201 | SEQ ID NO: 202 | SEQ ID NO: 224 |
| SEQ ID NO: 96 | | | |
| 4A8 VL V97K | SEQ ID NO: 201 | SEQ ID NO: 202 | SEQ ID NO: 225 |
| SEQ ID NO: 97 | | | |
| 4A8 VL N52S, N94S | SEQ ID NO: 201 | SEQ ID NO: 205 | SEQ ID NO: 213 |
| (SEQ ID NO: 109) | | | |
| 4A8 VL D41G, N52S, N94S | SEQ ID NO: 201 | SEQ ID NO: 205 | SEQ ID NO: 213 |
| (SEQ ID NO: 110) | | | |

Residue positions of substitutions defined according to Kabat.

In some aspects, the human antibody molecule can comprise:
a heavy chain CDR1, CDR2, and CDR3 of:
   SEQ ID NO: 176, SEQ ID NO: 186, and SEQ ID NO: 194, respectively;
   SEQ ID NO: 177, SEQ ID NO: 186, and SEQ ID NO: 194, respectively;
   SEQ ID NO: 178, SEQ ID NO: 186, and SEQ ID NO: 194, respectively;
   SEQ ID NO: 179, SEQ ID NO: 186, and SEQ ID NO: 194, respectively;
   SEQ ID NO: 180, SEQ ID NO: 186, and SEQ ID NO: 194, respectively;
   SEQ ID NO: 181, SEQ ID NO: 186, and SEQ ID NO: 194, respectively;
   SEQ ID NO: 182, SEQ ID NO: 186, and SEQ ID NO: 194, respectively;
   SEQ ID NO: 183, SEQ ID NO: 186, and SEQ ID NO: 194, respectively;
   SEQ ID NO: 184, SEQ ID NO: 186, and SEQ ID NO: 194, respectively;
   SEQ ID NO: 185, SEQ ID NO: 186, and SEQ ID NO: 194, respectively;
   SEQ ID NO: 175, SEQ ID NO: 187, and SEQ ID NO: 194, respectively;
   SEQ ID NO: 175, SEQ ID NO: 188, and SEQ ID NO: 194, respectively;
   SEQ ID NO: 175, SEQ ID NO: 189, and SEQ ID NO: 194, respectively;
   SEQ ID NO: 175, SEQ ID NO: 190, and SEQ ID NO: 194, respectively;
   SEQ ID NO: 175, SEQ ID NO: 191, and SEQ ID NO: 194, respectively;
   SEQ ID NO: 175, SEQ ID NO: 192, and SEQ ID NO: 194, respectively;
   SEQ ID NO: 175, SEQ ID NO: 186, and SEQ ID NO: 195, respectively;
   SEQ ID NO: 175, SEQ ID NO: 186, and SEQ ID NO: 196, respectively;
   SEQ ID NO: 175, SEQ ID NO: 186, and SEQ ID NO: 197, respectively;
   SEQ ID NO: 175, SEQ ID NO: 186, and SEQ ID NO: 198, respectively;
   SEQ ID NO: 175, SEQ ID NO: 186, and SEQ ID NO: 199, respectively; or
   SEQ ID NO: 175, SEQ ID NO: 186, and SEQ ID NO: 200, respectively; and
a light chain CDR1, CDR2, and CDR3 of SEQ ID NO: 201, SEQ ID NO: 202, and SEQ ID NO: 210, respectively.

In some aspects, the human antibody molecule can comprise:
a heavy chain CDR1, CDR2, and CDR3 of SEQ ID NO: 175, SEQ ID NO: 186, and SEQ ID NO: 194, respectively, and
a light chain CDR1, CDR2, and CDR3 of:
   SEQ ID NO: 201, SEQ ID NO: 203, and SEQ ID NO: 210, respectively;
   SEQ ID NO: 201, SEQ ID NO: 204, and SEQ ID NO: 210, respectively;
   SEQ ID NO: 201, SEQ ID NO: 205, and SEQ ID NO: 210, respectively;
   SEQ ID NO: 201, SEQ ID NO: 206, and SEQ ID NO: 210, respectively;
   SEQ ID NO: 201, SEQ ID NO: 207, and SEQ ID NO: 210, respectively;
   SEQ ID NO: 201, SEQ ID NO: 208, and SEQ ID NO: 210, respectively;
   SEQ ID NO: 201, SEQ ID NO: 209, and SEQ ID NO: 210, respectively;
   SEQ ID NO: 201, SEQ ID NO: 202, and SEQ ID NO: 211, respectively;
   SEQ ID NO: 201, SEQ ID NO: 202, and SEQ ID NO: 212, respectively;
   SEQ ID NO: 201, SEQ ID NO: 202, and SEQ ID NO: 213, respectively;
   SEQ ID NO: 201, SEQ ID NO: 202, and SEQ ID NO: 214, respectively;
   SEQ ID NO: 201, SEQ ID NO: 202, and SEQ ID NO: 215, respectively;
   SEQ ID NO: 201, SEQ ID NO: 202, and SEQ ID NO: 216, respectively;
   SEQ ID NO: 201, SEQ ID NO: 202, and SEQ ID NO: 217, respectively;
   SEQ ID NO: 201, SEQ ID NO: 202, and SEQ ID NO: 218, respectively;
   SEQ ID NO: 201, SEQ ID NO: 202, and SEQ ID NO: 219, respectively;
   SEQ ID NO: 201, SEQ ID NO: 202, and SEQ ID NO: 220, respectively;
   SEQ ID NO: 201, SEQ ID NO: 202, and SEQ ID NO: 221, respectively;
   SEQ ID NO: 201, SEQ ID NO: 202, and SEQ ID NO: 222, respectively;
   SEQ ID NO: 201, SEQ ID NO: 202, and SEQ ID NO: 223, respectively;
   SEQ ID NO: 201, SEQ ID NO: 202, and SEQ ID NO: 224, respectively; or
   SEQ ID NO: 201, SEQ ID NO: 202, and SEQ ID NO: 225, respectively.

In some aspects, the human antibody molecule can comprise:
a heavy chain CDR1, CDR2, and CDR3 of:
   SEQ ID NO: 182, SEQ ID NO: 192, and SEQ ID NO: 194, respectively;
   SEQ ID NO: 182, SEQ ID NO: 192, and SEQ ID NO: 197, respectively;
   SEQ ID NO: 175, SEQ ID NO: 186, and SEQ ID NO: 197, respectively; or
   SEQ ID NO: 182, SEQ ID NO: 192, and SEQ ID NO: 195, respectively; and
a light chain CDR1, CDR2, and CDR3 of SEQ ID NO: 201, SEQ ID NO: 205, and SEQ ID NO: 213, respectively.

The human antibody molecules can comprise the heavy chain CDR1 comprising the amino acid sequence of SEQ ID NO: 182, the heavy chain CDR2 comprising the amino acid sequence of SEQ ID NO: 192, the heavy chain CDR3 comprising the amino acid sequence of SEQ ID NO: 195, the light chain CDR1 comprising the amino acid sequence of SEQ ID NO: 201, the light chain CDR2 comprising the amino acid sequence of SEQ ID NO: 205, and the light chain CDR3 comprising the amino acid sequence of SEQ ID NO: 213.

As provided in Table 19 and FIGS. 4A and 4B, SEQ ID NO: 167 and 168 represent a consensus heavy chain variable region and light chain variable region, respectively (SEQ ID NO: 167=“consensus VH” and SEQ ID NO: 168=“consensus VL”), of the disclosed human antibody molecules. Thus, the disclosed human antibody molecules can comprise the heavy chain variable region comprising the amino acid sequence of

EVQLLESGGGLVQPGGSLRLSCAASGFTFSSX₁X₂X₃SWVRQAPGKGLEWV
SAX₄SX₅X₆X₇RX₈TYYADSVKGRFTISRDNSRNTLYLQMNSLRAEDTAVYY
CAX₉QX₁₀X₁₁X₁₂WQGILVTVSS

wherein: X₁ is S, Q, H, L, W, or Y; X₂ is T or A; X₃ is M, Q, D, H, or W; X₄ is I or H; X₅ is G or D; X₆ is R, S, or Q; X₇ is G or D; X₈ is N or S; X₉ is I or K; X₁₀ is M, A, Q, or K; X₁₁ is G or D; and X₁₂ is Y, S, or K as provided in SEQ ID NO: 167 and the light chain variable region comprising the amino acid sequence of

QSALTQPPSASGSPGQSVTISCIGTSSDVGGYNYVSWYQQHPDKAPKLMI
YX₁₃VX₁₄X₁₅X₁₆PSGVPDRFSGSKSGNTASLTVSGLQAEDEADYYCSSX₁₇
AGX₁₈NX₁₉FGX₂₀FGGGTKLTVL

wherein: X₁₃ is E or D; X₁₄ is N, D, or S; X₁₅ is K, A, D, or H; X₁₆ is R or Q; X₁₇ is Y or A; X₁₈ is N, A, S, K, L, W, or Y; X₁₉ is N, Q, D, H, K, L, or Y; and X₂₀ is V, A, or K as provided in SEQ ID NO: 168. The underlined residues represent the consensus CDRs as disclosed above.

The human antibody molecules can comprise the heavy chain variable region comprising the amino acid sequence of

EVQLLESGGGLVQPGGSLRLSCAASGFTFSSSTX₂₁SWVRQX₂₂PGKGLEWV
SAISGX₂₃GX₂₄X₂₅TYYADSVKGRFTISRDNSX₂₆NTLYLQMNSLRAEDTAV
YYCAX₂₇QX₂₈GYWGQGILVTVSS

wherein: X₂₁ is M or Q; X₂₂ is A or P; X₂₃ is R or S; X₂₄ is R or G; X₂₅ is N or S; X₂₆ is R or K; X₂₇ is I or K; and X₂₈ is M, K, or A as provided in SEQ ID NO: 226 and the light chain variable region comprising the amino acid sequence of

QSALTQPPSASGSPGQSVTISCIGTSSDVGGYNYVSWYQQHPX₂₉KAPKLM
IYEVX₃₀KRPSGVPDRFSGSKSGNTASLTVSGLQAEDEADYYCSSYAGX₃₁N
NFGVFGGGTKLTVL

wherein: X₂₉ is D or G; X₃₀ is N or S; and X₃₁ is N or S as provided in SEQ ID NO: 227. The underlined residues represent the consensus CDRs as disclosed above.

The human antibody molecules can comprise:
a) the heavy chain variable region comprising the amino acid sequence of SEQ ID NO: 98 and the light chain variable region comprising the amino acid sequence of SEQ ID NO: 109 or 110;
b) the heavy chain variable region comprising the amino acid sequence of SEQ ID NO: 99 and the light chain variable region comprising the amino acid sequence of SEQ ID NO: 109 or 110;
c) the heavy chain variable region comprising the amino acid sequence of SEQ ID NO: 100 and the light chain variable region comprising the amino acid sequence of SEQ ID NO: 109 or 110;
d) the heavy chain variable region comprising the amino acid sequence of SEQ ID NO: 101 and the light chain variable region comprising the amino acid sequence of SEQ ID NO: 109 or 110;
e) the heavy chain variable region comprising the amino acid sequence of SEQ ID NO: 102 and the light chain variable region comprising the amino acid sequence of SEQ ID NO: 109 or 110;
f) the heavy chain variable region comprising the amino acid sequence of SEQ ID NO: 103 and the light chain variable region comprising the amino acid sequence of SEQ ID NO: 109 or 110;
g) the heavy chain variable region comprising the amino acid sequence of SEQ ID NO: 104 and the light chain variable region comprising the amino acid sequence of SEQ ID NO: 109 or 110;
h) the heavy chain variable region comprising the amino acid sequence of SEQ ID NO: 105 and the light chain variable region comprising the amino acid sequence of SEQ ID NO: 109 or 110;
i) the heavy chain variable region comprising the amino acid sequence of SEQ ID NO: 106 and the light chain variable region comprising the amino acid sequence of SEQ ID NO: 109 or 110;
j) the heavy chain variable region comprising the amino acid sequence of SEQ ID NO: 107 and the light chain variable region comprising the amino acid sequence of SEQ ID NO: 109 or 110;
k) the heavy chain variable region comprising the amino acid sequence of SEQ ID NO: 108 and the light chain variable region comprising the amino acid sequence of SEQ ID NO: 109 or 110;
l) the heavy chain variable region comprising the amino acid sequence of SEQ ID NO: 162 and the light chain variable region comprising the amino acid sequence of SEQ ID NO: 109 or 110;
m) the heavy chain variable region comprising the amino acid sequence of SEQ ID NO: 163 and the light chain variable region comprising the amino acid sequence of SEQ ID NO: 109 or 110;
n) the heavy chain variable region comprising the amino acid sequence of SEQ ID NO: 164 and the light chain variable region comprising the amino acid sequence of SEQ ID NO: 109 or 110;
o) the heavy chain variable region comprising the amino acid sequence of SEQ ID NO: 165 and the light chain variable region comprising the amino acid sequence of SEQ ID NO: 109 or 110; or
p) the heavy chain variable region comprising the amino acid sequence of SEQ ID NO: 166 and the light chain variable region comprising the amino acid sequence of SEQ ID NO: 109 or 110.

In some embodiments, the human antibody molecules comprise:
a) the heavy chain variable region comprising the amino acid sequence of SEQ ID NO: 108 and the light chain variable region comprising the amino acid sequence of SEQ ID NO: 110;
b) the heavy chain variable region comprising the amino acid sequence of SEQ ID NO: 162 and the light chain variable region comprising the amino acid sequence of SEQ ID NO: 110;
c) the heavy chain variable region comprising the amino acid sequence of SEQ ID NO: 163 and the light chain variable region comprising the amino acid sequence of SEQ ID NO: 110;
d) the heavy chain variable region comprising the amino acid sequence of SEQ ID NO: 164 and the light chain variable region comprising the amino acid sequence of SEQ ID NO: 110;
e) the heavy chain variable region comprising the amino acid sequence of SEQ ID NO: 165 and the light chain variable region comprising the amino acid sequence of SEQ ID NO: 110; or
f) the heavy chain variable region comprising the amino acid sequence of SEQ ID NO: 166 and the light chain variable region comprising the amino acid sequence of SEQ ID NO: 110.

The human antibody molecules can comprise the heavy chain variable region comprising the amino acid sequence of SEQ ID NO: 108 and the light chain variable region comprising the amino acid sequence of SEQ ID NO: 110.

The disclosed human antibody molecules can comprise a human IgG1, IgG2, or IgG4 heavy chain constant region. In some embodiments, the human antibody molecule comprises a human IgG1 heavy chain constant region. Suitable human IgG1 heavy chain constant regions include, for example, the amino acid sequence of SEQ ID NO: 122 or 124. In some aspects, the human IgG1 heavy chain constant region comprises the amino acid sequence of SEQ ID NO: 122. In some aspects, the human IgG1 heavy chain constant region comprises the amino acid sequence of SEQ ID NO: 124. In some embodiments, the human antibody molecule comprises a human IgG2 heavy chain constant region. In some aspects, the human IgG2 heavy chain constant region comprises the amino acid sequence of SEQ ID NO: 120. In some embodiments, the human antibody molecule comprises a human IgG4 heavy chain constant region. Suitable human IgG4 heavy chain constant regions include, for example, the amino acid sequence of SEQ ID NO: 116 or 118. In some embodiments, the human IgG4 heavy chain constant region comprises the amino acid sequence of SEQ ID NO: 116. In some aspects, the human IgG4 heavy chain constant region comprises the amino acid sequence of SEQ ID NO: 118. In some embodiments the human IgG4 heavy chain constant region comprises an S228P substitution. In some embodiments the human IgG4 heavy chain constant region comprises M252Y, S254T and T256E substitutions. In some embodiments, the IgG4 constant region comprises deletion of the carboxyl-terminal lysine residue relative to the wild type IgG4.

The human antibody molecules can comprise a human lambda (λ) light chain constant region or a human kappa (κ) light chain constant region. In some embodiments, the human antibody molecule comprises a human lambda II (λ₂) light chain constant region.

The human antibody molecules can comprise a human IgG1 heavy chain constant region and a human lambda II light chain constant region. The human antibody molecules can comprise a human IgG2 heavy chain constant region and a human lambda II light chain constant region. The human antibody molecules can comprise a human IgG4 heavy chain constant region and a human lambda II light chain constant region.

The human antibody molecules can be a full-length antibody or an antigen-binding fragment thereof. Suitable antigen-binding fragments include, for example, an Fab fragment, an F(ab)₂ fragment, or a single chain antibody.

The disclosed human antibody molecules selectively antagonize human CXCR2, thereby inhibiting CXCL1- or CXCL5-induced activation of CXCR2. The disclosed human antibody molecules may also partially inhibit CXCL8-induced activation of CXCR2. The disclosed human antibody molecules may also exhibit one or more of the following properties:
a) inhibit CXCL1-induced calcium flux in an HTS002C—CHEMISCREEN™ human CXCR2 chemokine receptor calcium-optimized cell line with an IC₅₀ of 0.8 to 2.4 at a CXCL1 concentration of from 1.5 to 3.4 nM;
b) not substantially inhibit CXCL8-induced calcium flux in an HTS002C—CHEMISCREEN™ human CXCR2 chemokine receptor calcium-optimized cell line;
c) inhibit CXCL1 or CXCL5-induced β-arrestin recruitment in a Tango™ cell based assay with an IC₅₀ of from 0.08 to 0.5 nM at a concentration of from 1.5-3.4 nM for CXCL1 and from 47.7 to 150 nM for CXCL5; or
d) reduce airway neutrophilia in a subject with airway neutrophilia without causing severe, sustained neutropenia.

Pharmaceutical compositions comprising any of the herein disclosed human antibody molecules are also provided. In some embodiments, the pharmaceutical compositions can comprise any of the herein disclosed human antibody molecules in combination with a pharmaceutically acceptable carrier.

Also provided are nucleic acid molecules encoding any of the herein disclosed human antibody molecules. Exemplary polynucleotides which encode a human antibody or fragment thereof as described herein are provided as SEQ ID NOS: 233-247. Exemplary polynucleotides which encode human antibody heavy chain constant regions are provided as SEQ ID NOS:248-256.

Vectors comprising the herein disclosed nucleic acid molecules are also disclosed.

Further provided are cells transformed to express any of the herein disclosed human antibody molecules.

### Methods of Treatment and Uses

CXCR2 antagonists have been the subject of studies, including clinical trials, for a range of conditions which involve pathologies associated with neutrophilic and/or monocytic inflammation and certain cancers which express CXCR2 or in which there is an element of neutrophil suppression of an anti-cancer response. Small molecule antagonists of CXCR2 have, for example, been developed for:
(a) COPD (see, for example, Miller, B. et al., (2017). Late Breaking Abstract—“Danirixin (GSK1325756) improves respiratory symptoms and health status in mild to moderate COPD—results of a 1 year first time in patient study.” European Respiratory Journal, 50);
(b) influenza (see, for example, study NCT02469298 described on ClinicalTrials.gov entitled “Safety, Tolerability and Clinical Effect of Danirixin in Adults With Influenza”);
(c) bronchiectasis (see, for example, De Soyza et al., (2015) “A randomised, placebo-controlled study of the CXCR2 antagonist AZD5069 in bronchiectasis.” Eur Respir J, 46, 1021-32);
(d) cystic fibrosis (see, for example, Moss et al., (2013). “Safety and early treatment effects of the CXCR2 antagonist SB-656933 in patients with cystic fibrosis.” J Cyst Fibros, 12, 241-8);
(e) severe asthma (see, for example, Nair et al., (2012) “Safety and efficacy of a CXCR2 antagonist in patients with severe asthma and sputum neutrophils: a randomized, placebo-controlled clinical trial.” Clin Exp Allergy, 42, 1097-103); and
(f) prostate cancer (see, for example, study number NCT03177187 described on ClinicalTrials.gov entitled “Combination Study of AZD5069 and Enzalutamide”).

Additionally, there is evidence that antagonism of CXCR2 may be beneficial in chronic upper airway diseases such as chronic rhinosinusitis (see, for example, Tomassen et al., (2016) “Inflammatory endotypes of chronic rhinosinusitis based on cluster analysis of biomarkers.” J Allergy Clin Immunol, 137, 1449-1456 e4); in vascular diseases including ischemia-reperfusion injury (Stadtmann and Zarbock, (2012), “CXCR2: From Bench to Bedside.” Front Immunol, 3, 263) and coronary artery disease (see, for example, Joseph et al., (2017) “CXCR2 Inhibition—a novel approach to treating Coronary heart Disease (CICADA): study protocol for a randomised controlled trial.” Trials, 18, 473); in chronic pain (see, for example, Silva et al., (2017) “CXCL1/CXCR2 signaling in pathological pain: Role in peripheral and central sensitization.” Neurobiol Dis, 105, 109-116); in neuroinflammatory conditions (see, for example, Veenstra and Ransohoff, (2012) “Chemokine receptor CXCR2: physiology regulator and neuroinflammation controller?” J Neuroimmunol, 246, 1-9) including multiple sclerosis (see, for example, Pierson et al., (2018) “The contribution of neutrophils to CNS autoimmunity.” Clin Immunol, 189, 23-28) and Alzheimer's disease (see, for example, Liu et al., (2014) “Neuroinflammation in Alzheimer's disease: chemokines produced by astrocytes and chemokine receptors.” Int J Clin Exp Pathol, 7, 8342-55); in alcoholic and non-alcoholic steatohepatitis (see, for example, French et al., (2017) “The role of the IL-8 signaling pathway in the infiltration of granulocytes into the livers of patients with alcoholic hepatitis.” Exp Mol Pathol, 103, 137-140 and Ye et al., (2016) “Lipocalin-2 mediates non-alcoholic steatohepatitis by promoting neutrophil-macrophage crosstalk via the induction of CXCR2.” J Hepatol, 65, 988-997); in pancreatitis (see, for example, Steele et al., (2015) “CXCR2 inhibition suppresses acute and chronic pancreatic inflammation.” J Pathol, 237, 85-97); in diabetes (see, for example, Citro et al., (2015) “CXCR1/2 inhibition blocks and reverses type 1 diabetes in mice.” Diabetes, 64, 1329-40); and in multiple types of cancer (see, for example, Liu et al., (2016) “The CXCL8-CXCR1/2 pathways in cancer.” Cytokine Growth Factor Rev, 31, 61-71). Behçet's disease is characterized by neutrophil activation and has also been linked to CXCR2 (Qiao et al., “CXCR2 Expression on neutrophils is upregulated during the relapsing phase of ocular Behcet disease” Curr Eye Res. 2005; 30: 195-203).

The methods comprise administering to the subject a therapeutically effective amount of any of the herein disclosed human antibody molecules or the herein disclosed pharmaceutical compositions to treat or prevent the inflammation condition described herein. In some embodiments, the human antibody molecules or pharmaceutical compositions comprising the same are administered in a therapeutically effective amount to treat airway neutrophilia, as determined for example by sputum neutrophil counts, or acute lung inflammation. In such embodiments, the subjects receiving the human antibody molecules or pharmaceutical compositions comprising the same have airway neutrophilia or acute lung inflammation. In some embodiments, the human antibody molecules or pharmaceutical compositions comprising the same are administered in a therapeutically effective amount to prevent airway neutrophilia or acute lung inflammation. In such embodiments, the subjects receiving the human antibody molecules or pharmaceutical compositions comprising the same are at risk of having airway neutrophilia or acute lung inflammation due to, for example, genetic predisposition or environmental factors, or were previously treated for having airway neutrophilia or acute lung inflammation and are receiving, or are set to receive, therapeutically effective doses of the disclosed human antibody molecules or pharmaceutical compositions as a maintenance medication (e.g. to maintain low levels of lung neutrophils).

Also provided is the disclosed human antibody molecules or the disclosed pharmaceutical compositions for use in the prevention or treatment of airway neutrophilia or acute lung inflammation, as is the use of any of the disclosed human antibody molecules or any of the disclosed pharmaceutical compositions in the manufacture of a medicament for the prevention or treatment of neutrophilia in a peripheral tissue, airway neutrophilia or acute or chronic lung inflammation.

The airway neutrophilia, acute lung inflammation, or both can be chronic obstructive pulmonary disease, severe neutrophilic asthma, or both.

In at least one experimental model, the antibodies described herein have been observed to inhibit the migration of eosinophils into lung in response to inflammatory stimuli. Accordingly, the antibodies are useful for treating inflammatory diseases characterized by eosinophilia, such as eosinophilic asthma, allergic rhinitis, skin conditions, fungal and parasitic infections, autoimmune diseases (such as inflammatory bowel diseases, neuromyelitis optica, bullous pemphigoid, autoimmune myocarditis, primary biliary cirrhosis, eosinophilic granulomatosis with polyangiitis (Churg-Strauss syndrome), some cancers, and bone marrow disorders.

Without being bound by theory, the ability of the present CXCR2 antibodies to block eosinophilic migration into the lungs is surprising because eosinophils lack the CXCR2 receptor. For inflammatory cells to migrate into a site, they must first cross through the lining of the blood vessels, which are made up of endothelial cells, which are known to express CXCR2. Accordingly, the antibodies are further able to inhibit cell migration through effects on the endothelial cells.

The ability to affect endothelial cells also indicates that the present antibodies may be able to affect angiogenesis and metastasis, which is important in cancer. Accordingly, provided herein is a method for the treatment of cancer.

### EXAMPLES

The following examples are provided to further describe some of the embodiments disclosed herein. The examples are intended to illustrate, not to limit, the disclosed embodiments.

### General Methods

### Generation of Plasmids for Antibody Production

Variable region amino acid sequences were backtranslated into DNA sequences prior to synthesis of the resulting DNA de novo. Synthesized heavy chain variable region genes were subcloned into an expression vector containing a polynucleotide sequence encoding a human IgG4 constant region comprising the core hinge stabilizing substitution S228P (SEQ ID NO: 118). Synthesized lambda light chain variable region genes were subcloned into an expression vector containing a polynucleotide sequence encoding the human lambda light chain constant region amino acid sequence (SEQ ID NO: 134). Synthesized kappa light chain variable region genes were subcloned into an expression vector containing a polynucleotide sequence encoding the human kappa light chain constant region amino acid sequence (SEQ ID NO: 135).

### Transient Expression of Antibodies Using the Expi293F™ System

Antibodies were produced by co-transfecting antibody heavy and light plasmids into Expi293™ cells (Life Technologies). For each 20 mL transfection, 3.6×10⁷ cells were required in 20 mL of Expi293™ Expression Medium. Transfections were carried out using ExpiFectamine™ 293 Reagent according to manufacturer's instructions.

Antibodies were harvested by centrifugation (3000×g for 20 minutes) between 72 hours and 84 hours post-transfection. Unless indicated otherwise, all antibodies were produced as human IgG4 incorporating the hinge stabilizing substitution S228P.

### Purification and Buffer Exchange of Antibodies

Antibodies were purified from harvested material from transient transfections using protein A resin (MabSelect™ SuRe™, GE Healthcare) according to manufacturer's instructions.

Following elution, antibodies were buffer exchanged from citric acid into Sorensen's PBS, pH 5.8 (59.5 mM KH₂PO₄, 7.3 mM Na₂HPO₄·2H₂0, 150 mM NaCl) using PD-10 desalting columns (52-1308-00 BB, GE Healthcare) containing 8.3 mL of Sephadex™ G-25 Resin.

Transient Transfection of CXCR Family Members into Expi293F™ Cells

Expi293F™ cells (Life Technologies) were transiently transfected, using the commercially available mammalian expression vector pTT5 (Durocher, 2002) containing a polynucleotide encoding either: human CXCR1 (SEQ ID NO: 133); human CXCR2 (SEQ ID NO: 125); human CXCR3 (SEQ ID NO: 128); human CXCR4 (SEQ ID NO: 129); human CXCR5 (SEQ ID NO: 130); human CXCR6 (SEQ ID NO: 131); human CXCR7 (SEQ ID NO: 132); or cynomolgus monkey CXCR2 (SEQ ID NO: 127).

For each 10 mL transfection, lipid-DNA complexes were prepared by diluting 10 μg of plasmid DNA in Opti-MEM™ I Reduced Serum Medium (Cat. no. 31985-062) to a total volume of 1.0 mL. 54 μL of ExpiFectamine™ 293 Reagent was diluted in Opti-MEM™ I medium to a total volume of 1.0 mL. Transfections were carried out according to manufacturer's instructions. The cells were incubated in a 37° C. incubator with a humidified atmosphere of 8% CO₂ in air on an orbital shaker rotating at 200 rpm. Approximately 18-24 hours post-transfection, the cell viability was evaluated and transfected cells harvested for use.

### Flow Cytometry Binding Assays Using Transiently Transfected Expi293F™ Cells

Harvested cells were resuspended in FACS buffer (1×PBS+0.5% (w/v) bovine serum albumin (BSA)+2 mM EDTA pH 7.2). Approximately 2×10⁵ cells were added per well in a 96-well round-bottomed plate. Cells were pelleted by centrifugation at 400 g for 5 mins at 4° C. and supernatants discarded. 25 μL of each test antibody or control antibody was added to the cells and incubated for 30 minutes at 4° C. Cells were washed twice in 100 μL FACS buffer followed by centrifugation at 400 g for 5 mins at 4° C.

For detection, 50 μL of secondary antibody (Table 2) was added to relevant samples. Commercially available antibodies that bound to transfected CXCR family members were used as positive controls to ensure that the cells were expressing the receptors. Cells were washed twice in 200 μL of FACS buffer followed by centrifugation at 400 g for 5 mins at 4° C. Before sample acquisition, cells were resuspended in 100 μL of FACS buffer. Samples were acquired using the high throughput sampler on a FACSCanto™ II cytometer (Beckton-Dickinson).

TABLE 2
Reagents used for flow cytometry analysis

| | Catalogue | Manufacturer | Recommended |
|---|---|---|---|
| Reagents | Number | Supplier | final dilutions |
| Anti-human CXCR1-FITC | FAB330F | R&D Systems | 1 in 10 |
| Anti-human CXCR2-FITC | 551126 | BD Biosciences | 1 in 5 |
| Anti-human CXCR3-FITC | 558047 | BD Biosciences | 1 in 10 |
| Anti-human CXCR4-FITC | 561735 | BD Biosciences | 1 in 10 |
| Anti-human CXCR5-FITC | 558112 | BD Biosciences | 1 in 10 |
| Anti-human CXCR6-PE | 356004 | BioLegend | 1 in 20 |
| Anti-human CXCR7-PE | 331104 | Bio Legend | 1 in 20 |
| Human IgG4, kappa (isotype | I4639-1MG | Sigma-Aldrich | 10 μg/mL |
| control) | | | |
| Anti-human IgG Fc specific- | F9512-2ML | Sigma-Aldrich | 1 in 200 |
| FITC | | | |
| Anti-Human Ig light chain λ | 316610 | BioLegend | 1 in 20 |
| Antibody - APC | | | |
| Anti-human CXCR2-APC | 320710 | BioLegend | 1 in 20 |
| Anti-human CXCR2-Alexa | FAB331R-100 | R&D Systems | 1 in 20 |
| Fluor ™ 647 | | | |
| Anti-human CXCR2 | 555932 | BD Biosciences | |
| Anti-human CXCR2 | MAB331 | R&D Systems | |
| 7AAD | 559925 | BD Biosciences | 1 in 50 |

### Human CXCR2 Cell-Based Potency Assay

Tango™ CXCR2 cell-based assay: The commercially available reporter cell line Tango™ CXCR2-bla U2OS was used to assess the ability of antibodies to inhibit CXCR2 activation by CXCL8 and CXCL1 (ThermoFisher Scientific, Australia). Cells were thawed, propagated, cultured and frozen according to the manufacturer's directions.

Preparation of Tango™ CXCR2-bla U2OS cells for use in cell-based assays: The manufacturer's protocol was altered to make use of 96-well plates instead of 384-well plates. Briefly, dividing cells were harvested one day prior to use. Cells were harvested and resuspended in assay medium (100% FreeStyle™ Expression Medium; Life Technologies; Cat #12338-018) at a viable cell density of 312,500 cells/mL. 128 μL of cell suspension was added per well in 96-well black-walled, clear bottom tissue culture-treated plates. Cells were incubated for 16-20 hrs at 37° C. in an atmosphere of 5% CO₂ prior to use in assays.

Tango™ assay procedure: Assays were set up and run as described in the manufacturer's protocol. The agonists used in both agonist and antagonist potency assays are provided in Table 3. For antagonist assays, agonists were used at concentrations in the EC₅₀-EC₈₀ range. Assays were read using a FlexStation® 3 (Molecular Devices) fluorescence plate reader configured with the parameters given in Table 4. The blue/green emission ratio for each well was calculated by dividing the blue emission values by the green emission values. All inhibition curves were fitted using a four-parameter dose-response using GraphPad Prism™ (Version 7.01).

TABLE 3
Agonists used in cell based potency assays

| | | | Tango ™ | | Calcium | |
|---|---|---|---|---|---|---|
| | | | Assay | | Flux Assay | |
| Human | Manu- | Catalogue | EC₅₀ (nM) | | EC₅₀ (nM) | |
| Agonist | facturer | Number | Mean | Range | Mean | Range |
| CXCL1 | Miltenyi | 130- | 2.3 | 1.5-3.4 | 2.62 | 0.7-6.7 |
| | Biotec | 108-974 | | | | |
| CXCL2 | R&D | 276-GB | 33.3 | 15-45.7 | 37.02 | insufficient |
| | Systems | | | | | data |
| CXCL3 | R&D | 277-GG | 11.9 | 6.0-22.7 | 12.25 | 10-18 |
| | Systems | | | | | |
| CXCL5 | R&D | 254-XB | 97.7 | 47.7-150 | 39.17 | 10.3-20.5 |
| | Systems | | | | | |
| CXCL6 | R&D | 333-GC | 9.4 | 5.6-12.4 | 28.94 | 26.8-32.4 |
| | Systems | | | | | |
| CXCL7 | R&D | 393-NP | 39.6 | 15.3-77.2 | 7.14 | 5.3-9 |
| | Systems | | | | | |
| CXCL8 | Miltenyi | 130- | 2.2 | 1.6-4.0 | 3.2 | 0.9-6.5 |
| | Biotec | 108-979 | | | | |

TABLE 4
Flexstation ® 3 fluorescence plate reader settings

| | Scan 1 (Blue) | Scan 2 (Green) |
|---|---|---|
| Excitation Filter | 409/20 nm | 409/20 nm |
| Emission Filter | 460/40 nm | 530/30 nm |

### Peripheral Blood Preparations

Whole human buffy coat, PBMC enriched fractions prepared from human peripheral blood buffy coat or cynomolgus monkey whole blood was used for analysis of antibody binding activity by flow cytometry. Briefly, blood was untreated or diluted 1:1 in sterile filtered room temperature phosphate-buffered saline (PBS). 30 mL samples were layered over 15 mL Lymphoprep™ (Stem Cell Technologies, cat #07851). Peripheral blood mononuclear cells (PBMC) were enriched by room temperature centrifugation at 700 g for 30 minutes with no braking. The PBMC layer was isolated and cells washed in PBS containing 2 mM EDTA with low speed centrifugation at 200 g, to remove platelet contamination. Whole blood or PBMC enriched cell fractions were resuspended in red blood cell lysing solution (BioLegend, 420301). Viable cell counts were determined and cells resuspended at 1×10⁷ cells per mL in FACS buffer (1×PBS+0.5% (w/v) BSA+2 mM EDTA pH 7.2).

Flow cytometry binding assays using whole blood or PBMC enriched preparations: Binding assays to detect CXCR2 on blood neutrophil populations using test antibodies were performed essentially as described for assays using transiently transfected Expi293F™ cells. Binding of antibodies to human CXCR2 on the surface of the neutrophils was measured using anti-CXCR2 antibody directly conjugated to the fluorophore allophycocyanin (APC). Matched isotype control antibodies were included for comparison. Cells were incubated with lineage specific antibodies and 2 μg/ml of APC conjugated anti-CXCR2 antibody or isotype control prepared in ice cold 3% BSA/PBS for a minimum of 30 minutes at 4° C. The commercial anti-CXCR2 antibody (clone 48331 (R&D Systems FAB331A)) was used as a positive control. Neutrophil populations were identified by characteristic granularity and size, and positive binding of commercially available antibodies against CD10 (Biolegend, clone Hl10a, catalogue number 312204). Cells were washed and fixed (BioLegend Fixation buffer, 420801) prior to analysis. The level of fluorescence on the cell surface was measured by flow cytometry. Using this method, test anti-CXCR2 antibodies were detected binding neutrophils from both human and cynomolgus monkey.

### Generation of Human CXCR2 Transgenic Mice

hCXCR2 knock-in mice were generated by use of homologous recombination in embryonic stem (ES) cells to insert the human CXCR2 into exon 1 of the mouse CXCR2 gene. The single coding exon of human CXCR2 was inserted into a vector with 5′ and 3′ arms homologous to the genomic location of the mouse coding exon of CXCR2, giving rise to a gene structure that coded for human CXCR2 but retained the mouse non-coding and regulatory elements. This vector was electroporated into C57Bl/6 mouse ES cells which were incorporated into C57Bl/6 mouse blastocysts and transplanted into pseudopregnant female mice. Pups were backcrossed to the parental C57Bl/6 strain and offspring screened by southern blot for germline transmission of the human CXCR2 gene. Mice heterozygous for human CXCR2 were intercrossed to give a homozygous human CXCR2 line and phenotype was confirmed by analysis of binding of anti-mouse and anti-human CXCR2 antibodies by flow cytometry.

### Comparator Antibody and Small Molecule CXCR2 Antagonists

Antagonist 1 is antibody HY29GL as described in Int'l Pub. No. WO2015/169811 A2 (VH and VL of sequences 20 and 29 from that reference). Antagonist 2 is antibody CX1_5 as described in Int'l Pub. No. WO2014/170317 A1 (VH and VL of sequences 115 and 114 from that reference). Antagonist 3 is anti-CXCR2 antibody clone 48311 (R&D systems, Catalogue number MAB331-500). Antagonist 4 is anti-CXCR2 antibody clone 6C6 (BD Biosciences, Catalogue number 555932). Antagonist 5 is the small molecule CXCR2 antagonist Danirixin. Antagonist 6 is the small molecule CXCR2 antagonist SCH 527123.

### Example 1—Generation of Anti-CXCR2 Antibodies

Generation of Anti-CXCR2 Antibodies with Human Variable Regions

Transgenic rats engineered to express antibodies with human variable regions (as disclosed in Int'l Pub. No. WO2008/151081) were used to raise antibodies against human CXCR2. Briefly, animals were subjected to weekly genetic immunization using a plasmid which encoded the amino acid sequence of human CXCR2 (SEQ ID NO: 125) until antibody titres against human CXCR2 were obtained, as measured by flow cytometry using CXCR2 positive transiently transfected HEK-293 cells.

### Production and Expansion of Hybridomas Expressing Anti-CXCR2 Antibodies

To generate hybridomas which produced monoclonal antibodies to human CXCR2, splenocytes and/or lymph node cells from animals with the highest anti-CXCR2 titres were isolated and fused to mouse myeloma cells (ATCC, CRL-1580). Cells were plated at approximately 1×10⁵ cells/mL in flat bottom microtiter plates, followed by a two week incubation in selective medium (10% FCS and 1×HAT (Sigma)). Hybridomas were expanded by serial passage through four media changes in 96-well plates (96-well stages 1 to 4), then, where required, expanded into T25 and T75 flasks. During the hybridoma expansion process, supernatants were monitored for CXCR2 binding activity by cell-based ELISA (cELISA) on cells transiently transfected to express human CXCR2 or murine CXCR2 (SEQ ID NOs: 125 or 126, respectively). Bound antibodies were detected using a goat anti-rat IgG-HRP (Southern Biotech, #3030-05) secondary antibody.

A panel of hybridomas was generated using lymph nodes and spleen cells from the transgenic rats engineered to express human variable region sequences. A cellular ELISA (cELISA) was used to detect human CXCR2 binding activity in supernatants taken during the expansion process for the hybridomas (Table 5). Hybridomas that retained expression of antibodies that bound CXCR2 after several passages were selected for DNA sequencing.

TABLE 5
Binding of Hybridoma Supernatants to Human CXCR2 or
Mouse CXCR2 Transfected Cells as Determined by cELISA

cELISA using hybridoma supernatant from
rats with human variable regions

| | Human CXCR2 | Murine CXCR2 |
|---|---|---|
| CLONE | % Positive* | % Positive* |
| BKO-1A1 | 38% | 6% |
| BKO-1B10 | 75% | 4% |
| BKO-1C1 | 3% | 5% |
| BKO-1C6 | 88% | 5% |
| BKO-1D1 | 67% | 7% |
| BKO-1D5 | 46% | 6% |
| BKO-1D9 | 85% | 5% |
| BKO-1E3 | 3% | 6% |
| BKO-1H3 | 41% | 3% |
| BKO-2A3 | 3% | 3% |
| BKO-2B10 | 79% | 5% |
| BKO-2C2 | 77% | 5% |
| BKO-2D1 | 28% | 4% |
| BKO-2D8 | 100% | 7% |
| BKO-2E4 | 80% | 7% |
| BKO-2F6 | 11% | 3% |
| BKO-2G4 | 83% | 4% |
| BKO-2G7 | 75% | 3% |
| BKO-2G10 | 139% | 4% |
| BKO-2H4 | 2% | 3% |
| BKO-2H7 | 23% | 5% |
| BKO-3A9 | 2% | 4% |
| BKO-3C3 | 8% | 10% |
| BKO-3D3 | 3% | 6% |
| BKO-3D6 | 2% | 3% |
| BKO-3E3 | 107% | 3% |
| BKO-3F4 | 86% | 42% |
| BKO-3F5 | 5% | 5% |
| BKO-3F6 | 6% | 5% |
| BKO-3G11 | 61% | 7% |
| BKO-3H11 | 24% | 5% |
| BKO-4A4 | 32% | 7% |
| BKO-4A5 | 2% | 7% |
| BKO-4A8 | 110% | 5% |
| BKO-4A10 | 11% | 5% |
| BKO-4B2 | 107% | 6% |
| BKO-4B7 | 2% | 3% |
| BKO-4B11 | 103% | 7% |
| BKO-4C1 | 97% | 6% |
| BKO-4E8 | 53% | 7% |
| BKO-4F10 | 118% | 7% |
| BKO-4F11 | 77% | 8% |
| BKO-4G3 | 2% | 2% |
| BKO-4G4 | 93% | 3% |
| BKO-4H5 | 81% | 4% |
| BKO-4H6 | 108% | 4% |
| BKO-4H11 | 4% | 6% |
| BKO-5A5 | 66% | 5% |
| BKO-5B8 | 53% | 5% |
| BKO-5C4 | 101% | 5% |
| BKO-5E8 | 109% | 8% |
| BKO-5F9 | 2% | 4% |
| BKO-5F10 | 119% | 4% |
| BKO-5G6 | 103% | 4% |
| BKO-5G11 | 101% | 22% |
| BKO-5H1 | 4% | 7% |
| BKO-5H4 | 5% | 8% |
| BKO-6A1 | 60% | 6% |
| BKO-6A2 | 4% | 7% |
| BKO-6A3 | 8% | 7% |
| BKO-6B8 | 53% | 4% |
| BKO-6C2 | 3% | 6% |
| BKO-6C4 | 39% | 5% |
| BKO-6D10 | 2% | 4% |
| BKO-6E4 | 79% | 3% |
| BKO-6F3 | 44% | 6% |
| BKO-6G1 | 101% | 4% |
| BKO-6H1 | 52% | 5% |
| BKO-7A3 | 21% | 8% |
| BKO-7A9 | 2% | 4% |
| BKO-7B1 | 15% | 5% |
| BKO-7B2 | 84% | 4% |
| BKO-7C11 | 73% | 4% |
| BKO-7D8 | 67% | 6% |
| BKO-7D9 | 5% | 7% |
| BKO-7E4 | 4% | 6% |
| BKO-7E7 | 94% | 12% |
| BKO-7F3 | 95% | 7% |
| BKO-7F11 | 2% | 3% |
| BKO-7G1 | 21% | 3% |
| BKO-7G2 | 2% | 8% |
| BKO-7G10 | 104% | 26% |
| BKO-7H7 | 3% | 7% |
| BKO-7H8 | 105% | 7% |
| BKO-7H11 | 4% | 9% |
| BKO-8B6 | 95% | 6% |
| BKO-8C2 | 5% | 8% |
| BKO-8C4 | 124% | 6% |
| BKO-8D2 | 11% | 7% |
| BKO-8D4 | 38% | 7% |
| BKO-8F3 | 56% | 3% |
| BKO-8G3 | 114% | 6% |
| BKO-8H8 | 104% | 7% |
| BKO-8H10 | 81% | 8% |
| BKO-9A8 | 124% | 8% |
| BKO-9C3 | 56% | 7% |
| BKO-9C4 | 2% | 3% |
| BKO-9E7 | 2% | 3% |
| BKO-9G1 | 66% | 4% |
| BKO-9G6 | 34% | 8% |
| BKO-9H5 | 4% | 7% |
| BKO-10A2 | 25% | 5% |
| BKO-10B4 | 109% | 6% |
| BKO-10D1 | 2% | 6% |
| BKO-10D8 | 30% | 10% |
| BKO-10F3 | 7% | 4% |
| BKO-10G10 | 89% | 4% |
| BKO-10H6 | 6% | 6% |
| Positive control | 100% | 100% |
| Negative control | 4% | 8% |

*Fluorescence units relative to positive control (100%)

### Sequencing of Antibodies Produced by Hybridoma Cells

Antibody variable domains were isolated by reverse transcription polymerase chain reaction (RT-PCR) using RNA produced from the non-clonal hybridoma cell pellets as a template. RNA was isolated from the plates of hybridomas using a GENELUTE™ 96 well total RNA purification kit (Sigma #RTN9602, RTN9604) according to the manufacturer's protocol. For standard RT-PCR, RNA was reverse transcribed into cDNA using an oligo (dT) primer and an AccuScript PfuUltra® II RT-PCR kit (Agilent #600184). cDNA synthesis reactions were assembled according the manufacturer's protocol and cDNA synthesis carried out at 42° C. for 30 minutes. For use in 5′-Rapid Amplification of CDNA Ends (5′-RACE) PCR, RNA was reverse transcribed into cDNA using a SMARTer® RACE kit (Takara) according to the manufacturer's directions to give 5′-RACE ready cDNA.

Amplification of human antibody variable regions from the panel of hybridomas was performed by PCR using either PfuUltraII (Agilent) or Q5 high fidelity DNA polymerases (NEB) according to the manufacturer's directions. The hybridoma panel heavy chains were amplified using primer pairs specific to the rodent heavy chain constant region DNA sequence and the DNA sequences of the human heavy chain leader sequences. The hybridoma panel lambda light chain variable regions were similarly amplified using primer pairs specific to the human lambda constant region DNA sequence and the DNA sequences of the human lambda chain leader sequences.

The concentration of the resulting purified DNA was assessed using a Nanodrop spectrophotometer. Sanger sequencing of the PCR fragments was performed using oligos designed to bind internally in the heavy or light chain amplicons. The resulting DNA sequences were conceptually translated into amino acid sequences for further analysis prior to their use in full length antibody chain generation. Antibodies with unique amino acid sequences were selected for conversion to full-length human antibodies.

Recombinant Monoclonal Antibodies with Binding to Human CXCR2

Hybridomas selected from those which secreted antibody that bound CXCR2 (as described in Table 5) were sequenced to identify variable region DNA and amino acids using RT-PCR as described above. These antibody variable regions were then generated by gene synthesis and subcloned into mammalian expression vectors as described in General Methods. Antibodies were produced by co-expression of heavy and light chain plasmids in Expi293F™ cells and purified by protein A column chromatography as described in General Methods. Where several heavy and/or light chains were identified from the same hybridoma cells, each heavy chain was paired with each light chain and the resulting antibodies given suffix a, b, c, etc. Purified antibodies were desalted into Sorensen's PBS pH 5.8 and tested by flow cytometry for binding to Expi293F™ cells transfected with human CXCR2 or human CXCR1 and mock transfected Expi293F™ cells. 26 antibodies that bound human CXCR2 but not human CXCR1 or mock transfected Expi293F™ cells were identified from the hybridoma panel for further characterization. These antibody sequences are given in Table 6.

TABLE 6
Sequences of Antibodies with a Human Variable Region
That Bound Human CXCR2 but Not Closely Related Human
CXCR1 or Mock-Transfected Expi293F ™ Cells

| Antibody | | |
|---|---|---|
| Name | VH (SEQ ID NO) | VL (SEQ ID NO) |
| BKO-1A1 | BKO_1A1_VH | BKO_1A1_VL |
| | (SEQ ID NO: 1) | (SEQ ID NO: 2) |
| BKO-1B10 | BKO_1B10_VH | BKO_1B10_VL |
| | (SEQ ID NO: 3) | (SEQ ID NO: 4) |
| BKO-1D1 | BKO_1D1_VH | BKO_1D1_VL |
| | (SEQ ID NO: 5) | (SEQ ID NO: 6) |
| BKO-1H3 | BKO_1H3_VH | BKO_1H3_VL |
| | (SEQ ID NO: 7) | (SEQ ID NO: 8) |
| BKO-2D8 | BKO_2D8_VH | BKO_2D8_VL |
| | (SEQ ID NO: 9) | (SEQ ID NO: 10) |
| BKO-3A9_b | BKO_3A9_VH | BKO_3A9_L3_E03_VL |
| | (SEQ ID NO: 11) | (SEQ ID NO: 12) |
| BKO-3D6 | BKO_3D6_VH | BKO_3D6_L6_G06_VL |
| | (SEQ ID NO: 13) | (BKO_5H4_VL) |
| | | (SEQ ID NO: 14) |
| BKO-3F4 | BKO_3F4_VH | BKO_3F4_L11_A11_VL |
| | (SEQ ID NO: 15) | (SEQ ID NO: 16) |
| BKO-4A8 | BKO_4A8_VH | BKO_4A8_VL |
| | (SEQ ID NO: 17) | (SEQ ID NO: 18) |
| BKO-4F10 | BKO_4F10_VH | BKO_4F10_VL |
| | (SEQ ID NO: 19) | (SEQ ID NO: 20) |
| BKO-5E8 | BKO_5E8_H5_C05_VH | BKO_5E8_L3_C03_VL |
| | (SEQ ID NO: 21) | (SEQ ID NO: 22) |
| BKO-5G11 | BKO_5G11_VH | BKO_5G11_VL |
| | (SEQ ID NO: 23) | (SEQ ID NO: 24) |
| BKO-5G6_c | BKO_5G6_VH | BKO_5G6_L12_E12_VL |
| | (SEQ ID NO: 25) | (SEQ ID NO: 26) |
| BKO-6A1_b | BKO_6A1_H4_A04_VH | BKO_6A1_E10_VL |
| | (SEQ ID NO: 27) | (SEQ ID NO: 28) |
| BKO-6A2_a | BKO_6A2_H1_B01_VH | BKO_6A2_L6_A06_VL |
| | (SEQ ID NO: 29) | (SEQ ID NO: 30) |
| BKO_7C11 | BKO_7C11_H6_B06_VH | BKO_7C11_G01_VL |
| | (SEQ ID NO: 31) | (SEQ ID NO: 32) |
| BKO-7G10_a | BKO_7G10_H1_B01_VH | BKO_7G10_L6_E06_VL |
| | (SEQ ID NO: 33) | (SEQ ID NO: 34) |
| BKO-7H8_b | BKO_7H8_H3_C03_VH | BKO_7H8_L10_F10_VL |
| | (SEQ ID NO: 35) | (SEQ ID NO: 36) |
| BKO-8B6 | BKO_8B6_VH | BKO_8B6_VL |
| | (SEQ ID NO: 37) | (SEQ ID NO: 38) |
| BKO-8C4 | BKO_8C4_VH | BKO_8C4_VL |
| | (SEQ ID NO: 39) | (SEQ ID NO: 40) |
| BKO-8G3_b | BKO_8G3_H4_D04_VH | BKO_8G3_L1_G01_VL |
| | (SEQ ID NO: 41) | (SEQ ID NO: 42) |
| BKO-8H10 | BKO_8H10_VH | BKO_8H10_VL |
| | (SEQ ID NO: 43) | (SEQ ID NO: 44) |
| BKO-8H8_b | BKO_8H8_H5_E05_VH | BKO_8H8_L7_H08_VL |
| | (SEQ ID NO: 45) | (SEQ ID NO: 46) |
| BKO-9A8 | BKO_9A8_H3_F03_VH | BKO_9A8_L1_H02_VL |
| | (SEQ ID NO: 47) | (SEQ ID NO: 48) |
| BKO-9C3_a | BKO_9C3_H8_G08_VH | BKO_9C3_L1_F01_VL |
| | (SEQ ID NO: 49) | (SEQ ID NO: 50) |
| BKO-10G10 | BKO_10G10_VH | BKO_10G10_VL |
| | (SEQ ID NO: 51) | (SEQ ID NO: 52) |

### Example 2—Functional Characterization of Anti-CXCR2 Hits

### Binding to Cynomolgus Monkey CXCR2

All recombinant antibodies that bound human CXCR2 but not human CXCR1 or mock-transfected Expi293™ cells were tested for binding to cynomolgus CXCR2 (SEQ ID NO: 127) using Expi293F™ cells transiently transfected with a plasmid encoding cynomolgus CXCR2 protein as described herein. Antibodies with detectable levels of cynomolgus CXCR2 binding were characterized further.

### Binding to Other Human CXCR Family Members

Human and cynomolgus monkey CXCR2 cross-reactive antibodies were tested by flow cytometry for binding to other human CXCR family members—CXCR3, CXCR4, CXCR5, CXCR6 and CXCR7 using Expi293F™ cells transiently transfected with a plasmid encoding either human CXCR3 (SEQ ID NO: 128), CXCR4 (SEQ ID NO: 129), CXCR5 (SEQ ID NO: 130), CXCR6 (SEQ ID NO: 131) or CXCR7 (SEQ ID NO: 132). Antibodies that were not selective for CXCR2 were discounted from further analysis.

### Inhibition of CXCL8-Mediated Activation of Human CXCR2

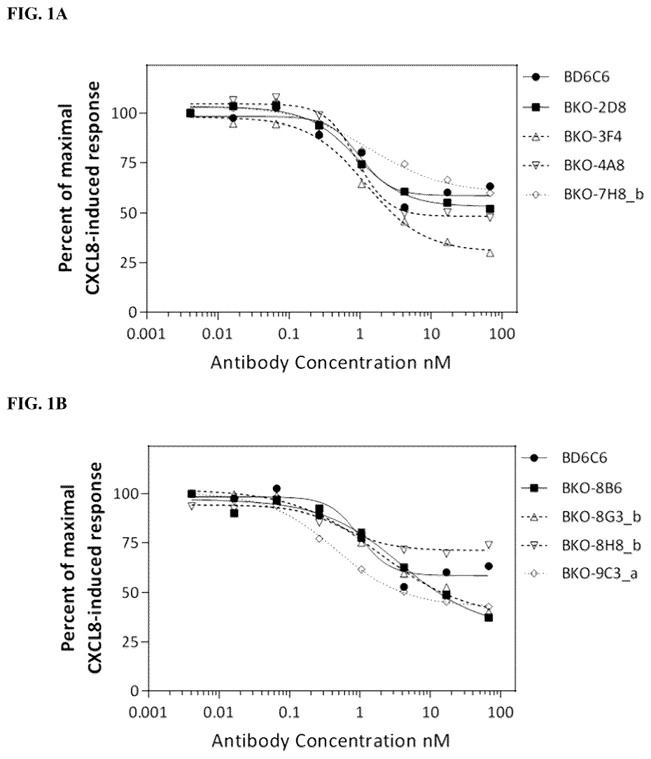

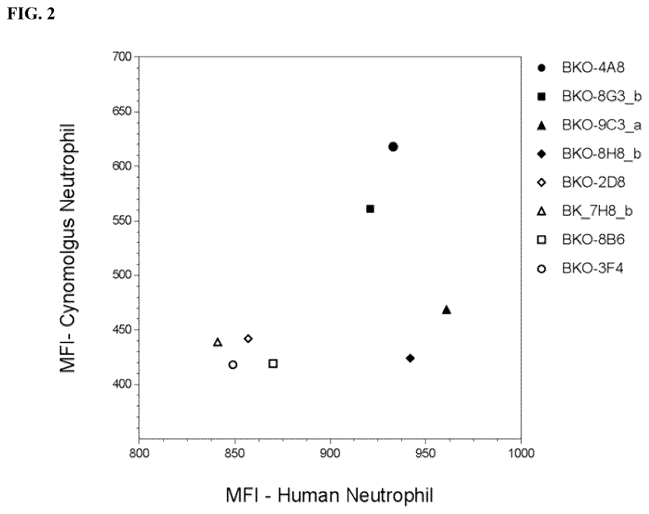

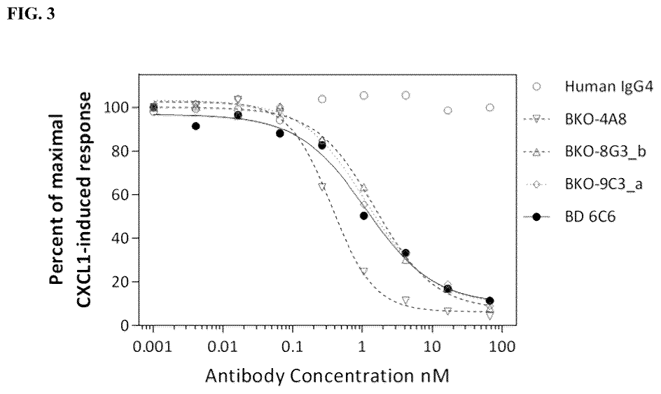

Antibodies were tested in a Tango™ CXCR2 cell-based assay using human CXCL8 as an agonist as described in General Methods. Eight antibodies inhibited CXCL8-induced activation of CXCR2, as shown in FIGS. 1A and 1B. A commercially available anti-CXCR2 antibody 6C6 (BD Biosciences; “BD6C6”) was used as a positive control in these assays.

### Binding of Antibodies to Human CXCR2 Expressed by Human PBMCs and Cynomolgus CXCR2 Expressed by Cynomolgus PBMCs

The eight antibodies that inhibited CXCL8-mediated activation of CXCR2 were tested for binding to native CXCR2 expressed on human and cynomolgus PBMCs. Two antibodies, BKO-4A8 and BKO-8G3_b, demonstrated high binding activity to both human and cynomolgus CXCR2 while a third, BKO-9C3_a, exhibited substantial levels of binding to human CXCR2 and above average levels of binding to cynomolgus CXCR2, as shown in FIG. 2.

### Inhibition of CXCL1-Mediated Activation of Human CXCR2

Antibodies BKO-4A8, BKO-8G3_b, and BKO-9C3_a were tested in a Tango™ CXCR2 cell-based assay using CXCL1 as an agonist as described in General Methods. Antibody BKO-4A8 was consistently the most potent of the antibodies tested in this format. Typical inhibition of CXCL1 activation of CXCR2 curves for these antibodies are shown below FIG. 3.

### Example 3—Amino Acid Sequence Optimization of Anti-CXCR2 Antibody BKO-4A8

Heavy and light chain variable region variants of the anti-CXCR2 antibody BKO-4A8 were constructed in an attempt to optimize the sequence of this molecule for stability and manufacturability.

Variants of antibody BKO-4A8, each comprising a single amino acid substitution, were produced as described below. A summary of these variants is provided in Table 7.

TABLE 7
Variants of BKO-4A8

| Antibody | | Variable region |
|---|---|---|
| Chain | Substitution | Sequence ID NO: |
| Heavy CDR1 | S32Q | 53 |
| | S32D | 136 |
| | S32H | 54 |
| | S32L | 55 |
| | S32W | 56 |
| | S32Y | 57 |
| | T33A | 58 |
| | M34Q | 59 |
| | M34D | 60 |
| | M34H | 61 |
| | M34W | 62 |
| | S35Q | 137 |
| | S35D | 138 |
| | S35K | 139 |
| Heavy CDR2 | A50S | 140 |
| | I51H | 63 |
| | G52aD | 64 |
| | R53S | 65 |
| | R53Q | 66 |
| | G54D | 67 |
| | R55Q | 141 |
| | R55D | 142 |
| | R55H | 143 |
| | N56S | 68 |
| Heavy CDR3 | I94K | 69 |
| | M96A | 70 |
| | M96S | 144 |
| | M96Q | 71 |
| | M96D | 145 |
| | M96H | 146 |
| | M96K | 72 |
| | M96L | 147 |
| | M96W | 148 |
| | M96Y | 149 |
| | G101D | 73 |
| | Y102S | 74 |
| | Y102Q | 150 |
| | Y102D | 151 |
| | Y102K | 75 |
| Light CDR2 | E50D | 76 |
| | V51D | 152 |
| | V51Y | 153 |
| | N52D | 77 |
| | N52S | 78 |
| | K53A | 79 |
| | K53D | 80 |
| | K53H | 81 |
| | R54Q | 82 |
| | R54D | 154 |
| Light CDR3 | Y91A | 83 |
| | Y91S | 155 |
| | Y91H | 156 |
| | N94A | 84 |
| | N94S | 85 |
| | N94H | 157 |
| | N94K | 86 |
| | N94L | 87 |
| | N94W | 88 |
| | N94Y | 89 |
| | N95aS | 158 |
| | N95aQ | 90 |
| | N95aD | 91 |
| | N95aH | 92 |
| | N95aK | 93 |
| | N95aL | 94 |
| | N95aW | 159 |
| | N95aY | 95 |
| | V97A | 96 |
| | V97S | 160 |
| | V97D | 161 |
| | V97K | 97 |

### Generation of Plasmids Encoding Antibody Variants by Site Directed Mutagenesis

Plasmids encoding antibody chains requiring single amino acid changes were prepared by mutagenic primer-directed replication of the plasmid strands using a high fidelity DNA polymerase. This process used supercoiled double-stranded plasmid DNA as the template and two complementary synthetic oligonucleotide primers both containing the desired mutation. The oligonucleotide primers, each complementary to opposite strands of the plasmid, were extended during the PCR cycling without primer displacement, resulting in copies of the mutated plasmid containing staggered nicks. Following PCR cycling, the PCR reaction was treated using the restriction enzyme DpnI. DpnI preferentially cuts the original vector DNA, leaving the newly synthesized strands intact.

To generate variants incorporating multiple amino acid substitutions that required mutagenic oligonucleotide primers of over forty bases in length, either the above process was repeated to introduce the changes sequentially or the genes were synthesized de novo by assembly of synthetic oligonucleotides. All construct DNA sequences were confirmed by Sanger DNA sequencing prior to use.

### Generation of Plasmids Encoding Antibody Variants by Polynucleotide Synthesis

Variable region amino acid sequences were backtranslated into DNA sequences using GeneOptimizer® technology prior to synthesis of the resulting DNA de novo by assembly of synthetic oligonucleotides (GeneArt, Germany). Polynucleotides encoding synthesized heavy chain variable regions paired with human IgG4 constant region comprising the core hinge stabilizing substitution S228P (SEQ ID NO: 118) were subcloned into an expression vector. Polynucleotides encoding synthesized lambda light chain variable region genes were subcloned into an expression vector encoding a human lambda light chain constant region (SEQ ID NO: 134).

### Binding of BKO-4A8 Variants to CXCR2

Each variant heavy chain was co-expressed with the parental light chain and vice versa. The resulting antibodies were purified and tested in CXCR2 binding assays relative to parental antibody BKO-4A8 as described herein. Antibody variants that had similar levels of binding to parental BKO-4A8 are shown in Table 8.

TABLE 8
BKO-4A8 point variants with similar
CXCR2 binding to parental BKO-4A8

| | | Variable region | Fold change in |
|---|---|---|---|
| Antibody | | Sequence ID | binding relative to |
| Chain | Substitution | NO: | BKO-4A8 |
| Heavy CDR1 | S32Q | 53 | 1.71 |
| | S32H | 54 | 0.86 |
| | S32L | 55 | 1.33 |
| | S32W | 56 | 0.83 |
| | S32Y | 57 | 1.33 |
| | T33A | 58 | 1.30 |
| | M34Q | 59 | 1.00 |
| | M34D | 60 | 1.60 |
| | M34H | 61 | 0.80 |
| | M34W | 62 | 1.17 |
| Heavy CDR2 | I51H | 63 | 1.80 |
| | G52aD | 64 | 1.18 |
| | R53S | 65 | 1.00 |
| | R53Q | 66 | 0.64 |
| | G54D | 67 | 1.00 |
| | N56S | 68 | 1.0 |
| Heavy CDR3 | I94K^{#} | 69 | 0.7 |
| | M96A | 70 | 1.5 |
| | M96Q | 71 | 1.5 |
| | M96K | 72 | 1.0 |
| | G101D | 73 | 1.0 |
| | Y102S | 74 | 1.5 |
| | Y102K | 75 | 1.3 |
| Light CDR2 | E50D | 76 | 2.0 |
| | N52D | 77 | 1.7 |
| | N52S | 78 | 1.0 |
| | K53A | 79 | 1.4 |
| | K53D | 80 | 1.4 |
| | K53H | 81 | 1.4 |
| | R54Q | 82 | 1.6 |
| Light CDR3 | Y91A | 83 | 1.7 |
| | N94A | 84 | 1.0 |
| | N94S | 85 | 1.0 |
| | N94K | 86 | 1.33 |
| | N94L | 87 | 0.88 |
| | N94W | 88 | 0.63 |
| | N94Y | 89 | 0.50 |
| | N95aQ | 90 | 1.0 |
| | N95aD | 91 | 2.3 |
| | N95aH | 92 | 1.3 |
| | N95aK | 93 | 1.0 |
| | N95aL | 94 | 1.7 |
| | N95aY | 95 | 1.7 |
| | V97A | 96 | 1.7 |
| | V97K | 97 | 1.0 |

^{#}is a framework residue which flanks Heavy CDR3

An alignment of the variable heavy chain sequences and variable light chain sequences of the above antibody variants are shown in FIG. 4A and FIG. 4B, respectively. The consensus variable heavy chain (“consensus VH”; SEQ ID NO: 167) and consensus variable light chain (“consensus VL”; SEQ ID NO: 168) are also provided in those figures.

BKO-4A8 Point Variants with Similar Potency to BKO-4A8 in Inhibiting CXCL1 or CXCL8 Activation of CXCR2

Sixteen antibody variants showing similar levels of CXCR2 binding to parental BKO-4A8 were tested in potency assays using CXCL1 or CXCL8 as an agonist as described in the General Methods. Twelve antibodies had similar potency to parental BKO-4A8 as shown in Table 9. Amino acid sequence alignments of these variants relative to the parental heavy and light chain sequences are provided in FIGS. 4A and 4B.

TABLE 9
BKO-4A8 Point Variants with Similar Potency to BKO-4A8 in
Inhibiting CXCL1- or CXCL8-mediated Activation of CXCR2

| | | | Tango Assay | |
|---|---|---|---|---|
| Antibody | | Variable region | IC₅₀ (nM) | |
| Chain | Substitution | SEQ ID NO: | CXCL1 | CXCL8 |
| BKO-4A8 | — | 17 | 0.177 | 0.112 |
| Heavy CDR1 | M34Q | 59 | 0.242 | 0.0776 |
| | M34H | 61 | 0.186 | 0.107 |
| Heavy CDR2 | N56S | 68 | 0.158 | 0.129 |
| Heavy CDR3 | I94K^{#} | 69 | 0.078 | 0.071 |
| | M96A | 70 | 0.140 | 0.126 |
| | M96Q | 71 | 0.330 | 0.140 |
| | M96K | 72 | 0.135 | 0.091 |
| Light CDR2 | N52S | 78 | 0.214 | 0.126 |
| Light CDR3 | N94S | 85 | 0.149 | 0.169 |
| | N94K | 86 | 0.123 | 0.113 |
| | N94Y | 89 | 0.164 | 0.105 |
| | N95aQ | 90 | 0.213 | 0.084 |

^{#}is a framework residue which flanks Heavy CDR3

### Combinatorial Variants of BKO-4A8

Three non-germline framework amino acids were substituted back to those seen in the closest human germline sequence—heavy chain R75K and I94K and light chain D41G. The substitution A40P was also introduced in framework 2 of the heavy chain.

A panel of combinatorial variants comprising two or more amino acid substitutions was designed to examine whether it was possible to further optimize these sequences. Table 10 describes a total of two light chain variants and eleven heavy chain variants which were produced. Amino acid sequence alignments with the parental BKO-4A8 variable heavy chain and variable light chain are illustrated in FIGS. 5A and 5B, respectively. The consensus variable heavy chain (“consensus VH”; SEQ ID NO: 226) and consensus variable light chain (“consensus VL”; SEQ ID NO: 227) are also provided in those figures.

TABLE 10
Combinatorial Variants of BKO-4A8

| | | Substitutions (vs BKO-4A8) |
|---|---|---|
| | | (Relative to parental HC variable region of |
| | | SEQ ID NO: 17 or parental LC variable |
| Chain | Variant: | region of SEQ ID NO: 18) |
| Heavy | 1 | M34Q, N56S |
| | | (SEQ ID NO: 98) |
| | 2 | M34Q, A40P, N56S |
| | | (SEQ ID NO: 99) |
| | 3 | M34Q, A40P, N56S, R75K |
| | | (SEQ ID NO: 100) |
| | 4 | M34Q, A40P, N56S, M96K |
| | | (SEQ ID NO: 101) |
| | 5 | M34Q, A40P, N56S, R75K, M96K |
| | | (SEQ ID NO: 102) |
| | 6 | M34Q, A40P, N56S, R75K, I94K, M96K |
| | | (SEQ ID NO: 103) |
| | 7 | M34Q, A40P, N56S, I94K, M96K |
| | | (SEQ ID NO: 104) |
| | 8 | M34Q, N56S, M96K |
| | | (SEQ ID NO: 105) |
| | 9 | M34Q, N56S, R75K, M96K |
| | | (SEQ ID NO: 106) |
| | 10 | I94K, M96K |
| | | (SEQ ID NO: 107) |
| | 101 | M34Q, A40P, N56S, R75K, M96A |
| | | (SEQ ID NO: 108) |
| Light | b | N52S, N94S |
| | | (SEQ ID NO: 109) |
| | c | D41G, N52S, N94S |
| | | (SEQ ID NO: 110) |

Each heavy chain was co-expressed with each light chain to produce a sequence optimized antibody variant. Antibodies were purified and tested for CXCR2 binding activity as described in General Methods. Most of the combinatorial antibody variants retained similar CXCR2 binding activity to parental antibody BKO-4A8, as shown in Table 11.

TABLE 11
Summary of CXCR2 binding data EC₅₀ values using combinatorial variants

| | | | CXCR2 | Fold |
|---|---|---|---|---|
| Antibody | | | binding | Improvement |
| Name | VH change(s) | VL change(s) | EC₅₀ (nM) | over 4A8 WT |
| 1b | M34Q, N56S | N52S, N94S | 0.3 | 1.33 |
| | (SEQ ID NO: 98) | (SEQ ID NO: 109) | | |
| 1c | | D41G, N52S, N94S | 0.3 | 1.33 |
| | | (SEQ ID NO: 110) | | |
| 4A8 control | N/A | N/A | 0.4 | N/A |
| for VH1 | | | | |
| 2b | M34Q, A40P, N56S | N52S, N94S | 0.4 | 1.25 |
| | (SEQ ID NO: 99) | (SEQ ID NO: 109) | | |
| 2c | | D41G, N52S, N94S | 0.3 | 1.67 |
| | | (SEQ ID NO: 110) | | |
| 4A8 control | N/A | N/A | 0.5 | N/A |
| for VH2 | | | | |
| 3b | M34Q, A40P, N56S, | N52S, N94S | 0.1 | 4 |
| | R75K | (SEQ ID NO: 109) | | |
| 3c | (SEQ ID NO: 100) | D41G, N52S, N94S | 0.2 | 2 |
| | | (SEQ ID NO: 110) | | |
| 4A8 control | N/A | N/A | 0.4 | N/A |
| for VH3 | | | | |
| 4b | M34Q, A40P, N56S, | N52S, N94S | 0.4 | 1.25 |
| | M96K | (SEQ ID NO: 109) | | |
| 4c | (SEQ ID NO: 101) | D41G, N52S, N94S | 0.4 | 1.25 |
| | | (SEQ ID NO: 110) | | |
| 4A8 control | N/A | N/A | 0.5 | N/A |
| for VH4 | | | | |
| 5b | M34Q, A40P, N56S, | N52S, N94S | 0.4 | 1.25 |
| | R75K, M96K | (SEQ ID NO: 109) | | |
| 5c | (SEQ ID NO: 102) | D41G, N52S, N94S | 0.4 | 1.25 |
| | | (SEQ ID NO: 110) | | |
| 4A8 control | N/A | N/A | 0.5 | N/A |
| for VH5 | | | | |
| 6b | M34Q, A40P, N56S, | N52S, N94S | 0.4 | 1.00 |
| | R75K, I94K, M96K | (SEQ ID NO: 109) | | |
| 6c | (SEQ ID NO: 103) | D41G, N52S, N94S | 0.9 | 0.44 |
| | | (SEQ ID NO: 110) | | |
| 4A8 control | N/A | N/A | 0.4 | N/A |
| for VH6 | | | | |
| 7b | M34Q, A40P, N56S, | N52S, N94S | 0.8 | 0.63 |
| | I94K, M96K | (SEQ ID NO: 109) | | |
| 7c | (SEQ ID NO: 104) | D41G, N52S, N94S | 1 | 0.50 |
| | | (SEQ ID NO: 110) | | |
| 4A8 control | N/A | N/A | 0.5 | N/A |
| for VH7 | | | | |
| 8b | M34Q, N56S, M96K | N52S, N94S | N/A | N/A |
| | (SEQ ID NO: 105) | (SEQ ID NO: 109) | | |
| 8c | | D41G, N52S, N94S | N/A | N/A |
| | | (SEQ ID NO: 110) | | |
| 4A8 control | N/A | N/A | 0.6 | N/A |
| for VH8 | | | | |
| 9b | M34Q, N56S, R75K, | N52S, N94S | 0.3 | 3.00 |
| | M96K | (SEQ ID NO: 109) | | |
| 9c | (SEQ ID NO: 106) | D41G, N52S, N94S | 6.4 | 0.14 |
| | | (SEQ ID NO: 110) | | |
| 4A8 control | N/A | N/A | 0.9 | N/A |
| for VH9 | | | | |
| 10b | I94K, M96K | N52S, N94S | 0.3 | 1.67 |
| | (SEQ ID NO: 107) | (SEQ ID NO: 109) | | |
| 10c | | D41G, N52S, N94S | 0.5 | 1.00 |
| | | (SEQ ID NO: 110) | | |
| 4A8 control | N/A | N/A | 0.5 | N/A |
| for VH10 | | | | |

A further six heavy chain combinational variants comprising four or more amino acid substitutions were produced as provided in Table 12. Amino acid sequence alignments with the parental BKO-4A8 heavy chain are illustrated in FIG. 5A. Each heavy chain was co-expressed with light chain c (SEQ ID NO:110) to produce an antibody variant. Antibodies were purified and tested for CXCR2 binding activity as described in General Methods. CXCR2 binding activity was reduced in antibody 102c and antibody 106c.

TABLE 12
Summary of CXCR2 binding data EC₅₀
values using combinatorial variants

| | | | | Max |
|---|---|---|---|---|
| | | | | binding |
| | | CXCR2 | | (MFI |
| | | binding | Max | Percent |
| Antibody | | EC₅₀ | binding | of 4A8 |
| Name | VH change(s) | (nM) | (MFI) | WT) |
| 101c | M34Q, A40P, N56S, R75K, | 3.05 | 55048 | 89% |
| | M96A | | | |
| | (SEQ ID NO: 108) | | | |
| 102c | M34Q, A40P, N56S, R75K, | 8.70 | 32980 | 53% |
| | M96E | | | |
| | (SEQ ID NO: 162) | | | |
| 103c | M34Q, A40P, R53S, R55G, | 5.15 | 44445 | 72% |
| | N56S, R75K | | | |
| | (SEQ ID NO: 163) | | | |
| 4A8 | N/A | 1.58 | 62074 | N/A |
| Control for | | | | |
| 101c-103c | | | | |
| 104c | M34Q, A40P, R53S, R55G, | 6.45 | 45097 | 54% |
| | N56S, R75K, M96K | | | |
| | (SEQ ID NO: 164) | | | |
| 105c | M34Q, A40P, R53S, R55G, | 5.73 | 42741 | 52% |
| | N56S, R75K, M96A | | | |
| | (SEQ ID NO: 165) | | | |
| 106c | M34Q, A40P, R53S, R55G, | >100 | 11633 | N/A |
| | N56S, R75K, M96E | | | |
| | (SEQ ID NO: 166) | | | |
| 4A8 | N/A | 3.82 | 82797 | N/A |
| Control for | | | | |
| 104c-106c | | | | |

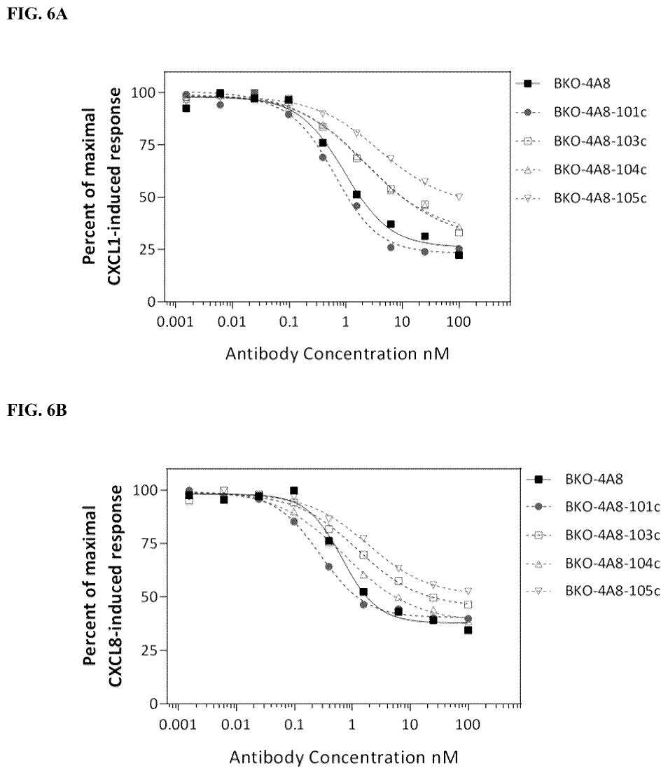

Antibodies were tested in potency assays using CXCL1 or CXCL8 as an agonist as described in the General Methods. Combinatorial antibody variants 103c, 104c, and 105c demonstrated reduced potency when compared to the parental antibody, while variant 101c demonstrated improved potency compared to the parental antibody as shown in Table 13 and FIGS. 6A and 6B.

TABLE 13
Summary of CXCR2 potency data EC₅₀ values using combinatorial variants

| | | CXCL8 | CXCL1 | CXCL8 Fold | CXCL1 Fold |
|---|---|---|---|---|---|
| Antibody | VH change(s) | IC₅₀ | IC₅₀ | Improvement | Improvement |
| Name | (VH SEQ ID NO) | (nM) | (nM) | over 4A8 WT | over 4A8 WT |
| 101c | M34Q, A40P, N56S, | 0.28 | 0.63 | 2.39 | 1.49 |
| | R75K, M96A | | | | |
| | (SEQ ID NO: 108) | | | | |
| 103c | M34Q, A40P, R53S, | 1.25 | 2.40 | 0.53 | 2.55 |
| | R55G, N56S, R75K | | | | |
| | (SEQ ID NO: 163) | | | | |
| 104c | M34Q, A40P, R53S, | 0.75 | 2.17 | 0.88 | 0.43 |
| | R55G, N56S, R75K, | | | | |
| | M96K | | | | |
| | (SEQ ID NO: 164) | | | | |
| 105c | M34Q, A40P, R53S, | 1.85 | 4.00 | 0.36 | 0.24 |
| | R55G, N56S, R75K, | | | | |
| | M96A | | | | |
| | (SEQ ID NO: 165) | | | | |
| 4A8 | N/A | 0.66 | 94 | N/A | N/A |
| Control | | | | | |

Antibody 101c comprising heavy chain variable region “101” and light chain variable region “c” was selected for evaluation in cell-based potency assays due to its favorable observed properties. Antibody 101c was renamed BKO-4A8-101c. BKO-4A8-101c comprised five heavy-and three light-chain optimizing substitutions. The comparison of BKO-4A8 with BKO-4A8-101c in cell-based potency assays measuring their ability to inhibit CXCL1 or CXCL8 mediated activation of CXCR2 suggested the sequence changes to optimize BKO-4A8-101c also increased its potency relative to parental BKO-4A8, as illustrated in FIGS. 6A and 6B.

### Example 5—Characterization of Anti-CXCR2 Antagonist Activity

### Characterization of BKO-4A8-101c Inhibition of Ligand Mediated β-Arrestin Recruitment

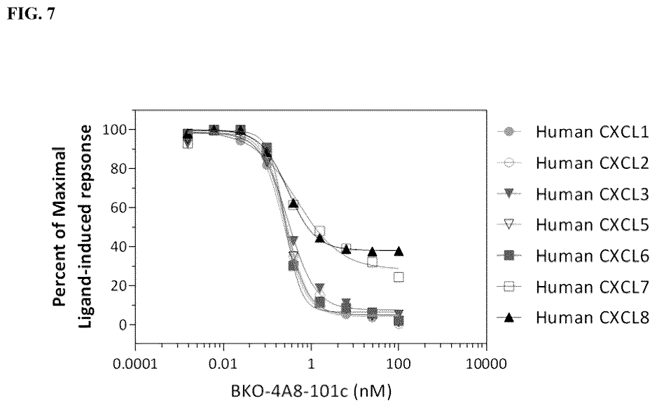

The Human CXCR2 Tango™ cell line was used to assess the ability of antibody BKO-4A8-101c to inhibit β-arrestin recruitment to agonist-activated CXCR2. All human ELR+chemokine CXCR2 ligands were tested in antagonist dose response assays using calculated EC₅₀ values of agonist. BKO-4A8-101c was able to inhibit CXCR2-mediated β-arrestin signaling induced by all ELR+CXCL chemokines, with comparable IC₅₀ values obtained for all agonists tested (Table 14). BKO-4A8-101c completely inhibited human CXCR2 activation by human CXCL1, 2, 3, 5, and 6 in a dose-dependent manner, but only partially inhibited CXCL7 and CXCL8 over the same dose range. Representative data from four independent experiments is shown in FIG. 7.

Without wishing to be bound by any proposed mechanism of action, it is proposed that the selective antagonist activity observed provides a therapeutic window to enable substantially complete inhibition of CXCL1- and CXCL5-mediated migration of neutrophils from the circulation into tissue without substantially affecting the migration of neutrophils mediated by CXCL8 from the bone marrow to the circulation. Partial inhibition of CXCL8-mediated β-arrestin in the reporter assay demonstrates that the β-arrestin-mediated receptor internalization pathway is functional.

TABLE 14
BKO-4A8-101c CXCR2 antagonist activity in a ligand-mediated
β-arrestin reporter assay (n = 7-13)

| | | | Maximal |
|---|---|---|---|
| | IC₅₀ nM | | Inhibition % |
| Hu Ligands | Mean | Range | Mean |
| Hu CXCL1 | 0.27 | 0.08-0.42 | 98 |
| Hu CXCL2 | 0.36 | 0.23-0.44 | 100 |
| Hu CXCL3 | 0.39 | 0.30-0.46 | 96 |
| Hu CXCL5 | 0.30 | 0.11-0.47 | 98 |
| Hu CXCL6 | 0.28 | 0.16-0.38 | 98 |
| Hu CXCL7 | 0.44 | 0.29-0.62 | 76 |
| Hu CXCL8 | 0.34 | 0.18-0.66 | 78 |

### Characterization of BKO-4A8-101c Inhibition of Ligand-Mediated Calcium Flux

One of the signaling pathways downstream of CXCR2 activation that has a role in cell chemotaxis is characterized by calcium mobilization (flux). The ability of BKO-4A8-101c to inhibit human CXCR2 ligand-induced calcium flux was tested in the commercially available HTS002C—CHEMISCREEN™ human CXCR2 chemokine receptor calcium-optimized cell line. All human ELR+chemokine CXCR2 ligands were tested in antagonist dose response assays using calculated agonist EC₅₀ values.

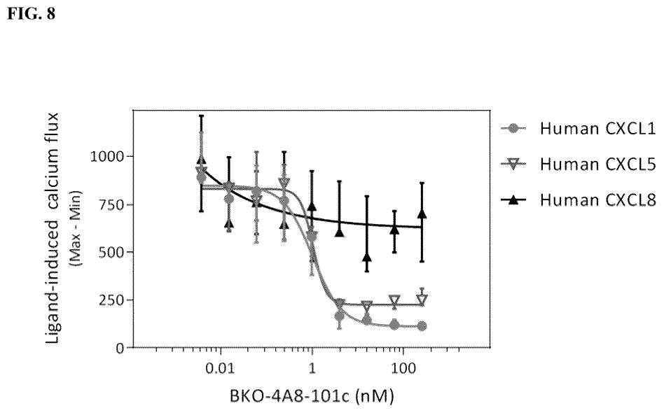

BKO-4A8-101c strongly inhibited calcium flux induced by human CXCL1, 2, 3, 5, and 6 in a dose-dependent manner, but only weakly inhibited CXCL7 and marginally inhibited CXCL8 over the same dose range, as shown in Table 15. Representative data is shown for CXCL1, CXCL5, and CXCL8 (FIG. 8).

The neutrophil chemotactic response is known to be mediated via CXCR2-activated calcium mobilization. Without wishing to be limited to any proposed mode of action, the selective antagonist activity provided by antibodies disclosed herein potentially provides a therapeutic window to enable substantially complete inhibition of the CXCL1- and CXCL5-mediated migration of neutrophils into lungs without substantially impacting CXCL8-mediated migration of neutrophils from bone marrow into the circulation. This may allow for blockade of neutrophil-mediated pathology at sites of chronic inflammation without necessarily impairing baseline neutrophil-mediated antimicrobial functions.

TABLE 15
Summary of mean IC₅₀ values of BKO-4A8-101c
for human ELR+ chemokines on human CXCR2
in a calcium flux assay (N = 4-9)

| | | | Maximal |
|---|---|---|---|
| | IC₅₀ nM | | Inhibition % |
| Hu Ligands | Mean | Range | Mean |
| Hu CXCL1 | 1.55 | 0.84-2.31 | 91 |
| Hu CXCL2 | 1.62 | 0.75-2.30 | 88 |
| Hu CXCL3 | 0.63 | 0.25-1.15 | 52 |
| Hu CXCL5 | 1.03 | N.A.^{a} | 81 |
| Hu CXCL6 | 1.71 | 1.57-2.00 | 81 |
| Hu CXCL7 | 2.14 | 0.47-5.5 | 43 |
| Hu CXCL8 | N.D.^{b} | — | 9 |

^{a}Insufficient data.
^{b}N.D. No inhibition or data that did not fit a four point dose response curve fit analysis.

### Example 6—In Vitro Binding and Functional Activity of Anti-CXCR2 Antibodies is Independent of Fc Region

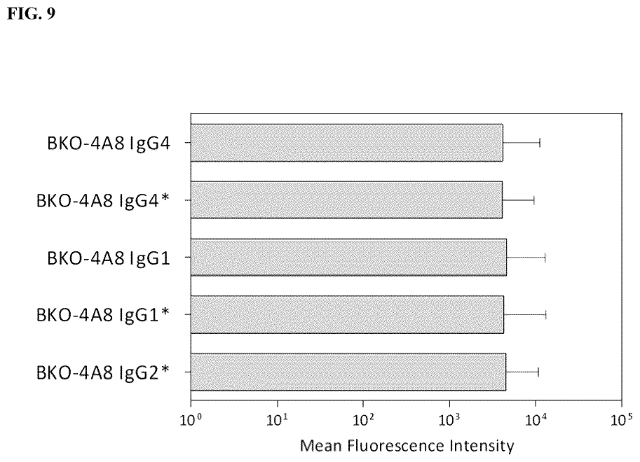

Variable regions from the BKO-4A8 heavy chain were formatted onto different human IgG constant regions as provided in the Table 16. The ability of purified antibodies to bind to human CXCR2 was routinely assessed on Expi293F™ cells transiently transfected to express human CXCR2 (SEQ ID NO: 125). Binding of BKO-4A8 and variants thereof was detected by incubation of fluorochrome-conjugate anti-human IgG light chain lambda antibody. The binding activity of antibodies tested, quantified as mean fluorescent intensity, was independent of the antibody Fc region as illustrated in FIG. 9.

TABLE 16
Heavy chain variants of BKO-4A8

| | Heavy Chain |
|---|---|
| BKO-4A8 heavy chain variant | SEQ ID NO |
| BKO-4A8 IgG4* | 115 |
| BKO-4A8 IgG4 | 117 |
| BKO-4A8 IgG2* | 119 |
| BKO-4A8 IgG1* | 121 |
| BKO-4A8 IgG1 | 123 |

*refers to a modified Fc

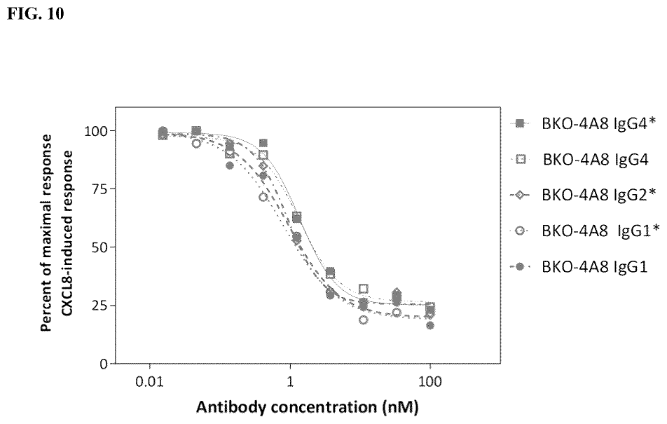

The commercially available reporter cell line Tango™ CXCR2-bla U2OS (ThermoFisher Scientific) was used to assess the ability of the anti-CXCR2 antibodies to inhibit β-arrestin recruitment to agonist-activated CXCR2. Agonists were provided at their assay EC₅₀ concentration for antagonist assays. The dose response curves demonstrated that functional activity of BKO-4A8 was not impacted by the sequence modification in the Fc region, with comparable antagonist activity evident for all antibodies evaluated, as illustrated in FIG. 10.

### Example 7—Specificity of Anti-CXCR2 Antibody BKO-4A8-101c

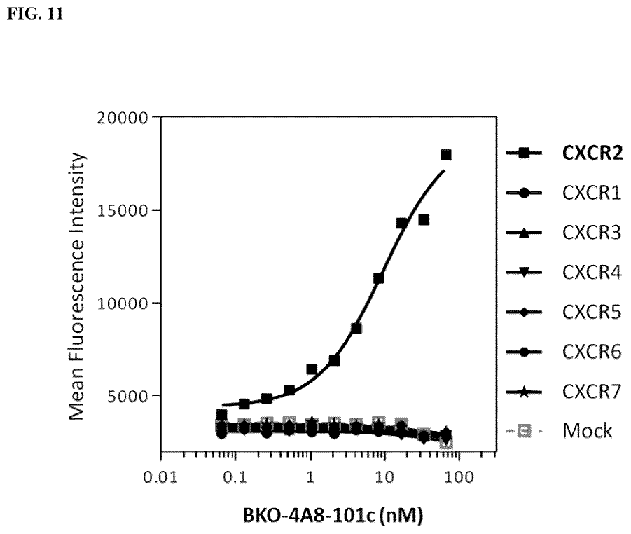

The specificity of the anti-CXCR2 antibody BKO-4A8-101c was tested by assessing binding activity on Expi293F™ cells transiently transfected to express closely related human CXCR family members as illustrated in FIG. 11. Sequences of the tested human CXCR family members are provided in Table 19. Binding of BKO-4A8-101c was detected by incubation of fluorochrome-conjugate anti-human IgG antibody. The anti-CXCR2 antibody BKO-4A8-101c was found to strongly and exclusively bind human CXCR2.

### Example 8—Flow Cytometry Binding Assays Using Primary Blood Cells

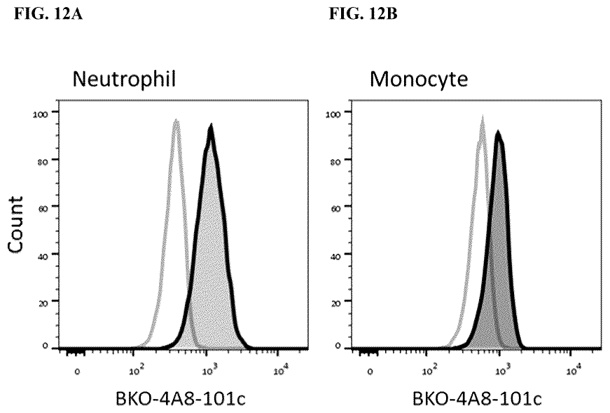

To further characterize the anti-CXCR2 antibody BKO-4A8-101c, the ability to bind CXCR2 on neutrophils was tested on anticoagulated human blood. Binding was measured using BKO-4A8-101c directly conjugated to fluorophore APC. Matched isotype control antibodies conjugated to APC were included for comparison. Cells were incubated with lineage-specific antibodies and 2 μg/mL of APC conjugated anti-CXCR2 antibody or isotype control. The level of fluorescence on the cell surface was measured by flow cytometry. Cellular debris and non-viable cells were excluded based on light scatter characteristics and incorporation of Zombie Violet fixable viability dye (BioLegend, 423113). Hematopoietic cell subsets were identified based on CD45 expression together with characteristic size (forward scatter, FSC) and granularity (side scatter, SSC) in association with expression of phenotypic markers as follows: T lymphocytes=CD3; B lymphocytes=CD20; Monocytes=CD14; and natural killer lymphocytes=CD56 (or CD3−CD20−CD16+lymphocytes). Granulocytes were identified according to size and granularity and the absence of binding of lineage specific markers: CD3, CD19, CD20, CD56, and CD14. Neutrophils were further distinguished by high levels of expression of CD16 and CD177, while eosinophils were identified based on Siglec-8 expression. Using this method BKO-4A8-101c bound neutrophils (FIG. 12A) and monocytes (FIG. 12B) from human blood. A commercial anti-CXCR2 antibody (BioLegend 5E8/CXCR2) was used as a positive control in the experiment.

### Example 9—The Ability of Purified Antibodies to Inhibit Agonist-Induced CXCR2-Mediated Functional Response Relative to Comparator Antibodies and Small Molecules

### Characterization of BKO-4A8-101c Inhibition of Ligand Mediated β-Arrestin Recruitment Relative to Comparator Reagents

The ability of BKO-4A8-101c to block ligand-activated CXCR2 signaling was compared with comparator antibodies and small molecules (see General Methods) in the Tango™ CXCR2 β-arrestin recruitment assay (Thermo), using the human ligands CXCL1, CXCL5, and CXCL8 at calculated EC₅₀ values.

BKO-4A8-101c inhibited CXCR2-mediated β-arrestin functional activity elicited by a panel of known CXCR2 ligands, with comparable IC₅₀ values for different ligands. The antibody demonstrated ligand selective inhibition of CXCR2-dependent β-arrestin recruitment with complete inhibition of CXCL1- and CXCL5 and partial inhibition of CXCL8-induced β-arrestin recruitment (range 70-80%) as shown in Table 17.

The potency of BKO-4A8-101c inhibition of CXCL1 and CXCL5-mediated β-arrestin signaling was similar or higher than that observed for other comparator antibodies and small molecules. Based on IC₅₀ values, BKO-4A8-101c was shown to be 2- to 39-fold more potent in inhibiting CXCL1- or CXCL5-ligand mediated β-arrestin activation than comparator CXCR2 antagonist antibodies or small molecules, but only an incomplete inhibitor of CXCL8-induced activation of CXCR2. Incomplete inhibition of CXCL8-mediated β-arrestin reporter activity demonstrates that the β-arrestin-mediated receptor internalization pathway is functional.

TABLE 17
Summary of mean IC₅₀ Values of CXCR2 antagonists for human ELR+
chemokines on human CXCR2 in a β-arrestin recruitment assay

| | CXCL1 | | CXCL5 | | CXCL8 | |
|---|---|---|---|---|---|---|
| | | Maximal | | Maximal | | Maximal |
| | IC₅₀ | Inhibition | IC₅₀ | Inhibition | IC₅₀ | Inhibition |
| Compound | (nM) | % | (nM) | % | (nM) | % |
| BKO-4A8- | 0.38 | 97 | 0.34 | 98 | 0.34 | 78 |
| 101c | | | | | | |
| Antagonist 1 | 1.20 | 98 | 0.80 | 98 | 0.58 | 97 |
| Antagonist 2 | 1.14 | 93 | 0.56 | 96 | 0.82 | 74 |
| Antagonist 3 | 14.80 | 85 | 8.12 | 96 | N.D.^{a} | 82 |
| Antagonist 4 | 1.47 | 96 | 0.75 | 99 | 1.01 | 94 |
| Antagonist 5 | 2.77 | 99 | 1.75 | 99 | 1.23 | 99 |
| Antagonist 6 | 0.71 | 99 | 0.29 | 97 | 1.22 | 98 |

^{a}N.D. No inhibition or data that did not fit a four point dose response curve fit analysis.

### Characterization of BKO-4A8-101c Inhibition of Calcium Mobilization

The ability of antagonists to inhibit CXCR2 activation of calcium mobilization induced by CXCL1 and CXCL8 was assessed in the commercially available HTS002C—CHEMISCREEN™ human CXCR2 chemokine receptor calcium-optimized stable cell line (Eurofins Pharma Discovery Services). CXC ligands were provided at their assay EC₅₀ concentration (see Table 3) for antagonist assays. The Fluo-4 NW calcium assay kit (Life Technologies) was used according to manufacturer's protocol for in-cell measurement of calcium mobilization, which was read using a FLIPR Tetra high-throughput cellular screening system (Molecular Devices). The peak response minus the basal response from each well was used to determine inhibition dose response curves fitting a four-parameter logistic equation and IC₅₀ values.

The ability of BKO-4A8-101c to block ligand-induced calcium flux was compared with comparator antibodies and small molecules (Table 18) using the human ligands CXCL1 and CXCL8 at calculated EC₅₀ values. The IC₅₀ values from at least 3 independent replicates are shown in Table 18. BKO-4A8-101c was an equivalent or more potent CXCL1 antagonist than comparator antibodies and small molecules and strongly inhibited calcium flux induced by human CXCL1. In contrast, BKO-4A8-101c and other comparator antibodies did not substantially inhibit calcium flux induced by CXCL8, while the comparator small molecule CXCR2 antagonists proved to be able to completely inhibit CXCL8 induced calcium flux in this assay. The neutrophil chemotactic response mediated via CXCR2 is dependent on calcium mobilization. Without wishing to be bound by any proposed mechanism of action, the selective antagonist activity observed with BKO-4A8-101c provides a therapeutic window to enable substantially complete inhibition of CXCL1- and CXCL5-mediated migration of neutrophils into tissue, without substantially impacting CXCL8-mediated migration from the bone marrow. This ligand selectivity may also affect the chemokine gradients that drive neutrophil chemotactic responses.

TABLE 18
Summary of IC₅₀ values of human CXCR2 antagonists for human
ELR+ chemokines on human CXCR2 in a calcium flux assay

| | CXCL1 | | CXCL8 | |
|---|---|---|---|---|
| | | Maximal | | Maximal |
| Compound | IC₅₀ (nM) | Inhibition % | IC₅₀ (nM) | Inhibition % |
| BKO-4A8- | 1.47 | 93 | N.D.^{a} | 9 |
| 101c | | | | |
| Antagonist 1 | N.D.^{a} | 28 | N.D.^{a} | 8 |
| Antagonist 2 | 1.02 | 79 | N.D.^{a} | 3 |
| Antagonist 3 | 30.74 | 37 | N.D.^{a} | 18 |
| Antagonist 4 | 1.29 | 93 | N.D.^{a} | 1 |
| Antagonist 5 | 48.94 | 95 | 115.12 | 97 |
| Antagonist 6 | 8.46 | 96 | 16.09 | 98 |

^{a}N.D. No inhibition or data that did not fit a four point dose response curve fit analysis.

### Example 10—Anti-CXCR2-Mediated Inhibition of Lung Neutrophilia in a Mouse Model of Severe Asthma

Sensitized mice challenged with intranasal house dust mite extract (HDM) develop an inflammatory profile with mixed pulmonary eosinophilia and neutrophilia associated with goblet cell hyperplasia. A severe asthma model has previously been reported using the small molecule CXCR2 antagonist SCH527123 at 10 and 30 mg/kg doses (as discussed in YOUNG, A., et al., The Effect of the CXCR1/2 Antagonist SCH257123 in a Mouse Model of Severe Asthma. Experimental Biology 2016 Meeting, 2016 San Diego, USA: The FASEB Journal, 1202.10) known to work in mouse models of neutrophilic inflammation (as discussed in CHAPMAN, R. W., et al., A novel, orally active CXCR1/2 receptor antagonist, Sch527123, inhibits neutrophil recruitment, mucus production, and goblet cell hyperplasia in animal models of pulmonary inflammation. J Pharmacol Exp Ther, (2007) 322, 486-93). Pre-treatment with 10 and 30 mg/kg SCH527123 only inhibited neutrophil cell numbers by a maximum of 30% in the BAL, which was not significantly different when compared to the house dust mite (HDM)-vehicle treated group (as discussed in Young et al., supra).

The anti-CXCR2 antibody BKO-4A8-mIgG1 (SEQ ID NOs: 113 and 114) was generated by formatting the variable heavy and light chains of BKO-4A8 onto an effector function-reduced mouse IgG1 constant region. Female hCXCR2 knock-in mice were subjected to sensitization with 50 μg HDM in complete Freund's adjuvant (CFA) administered by subcutaneous injection on day 0 and intranasal challenge with 50 mg HDM without CFA on day 14. Animals were treated with vehicle or BKO-4A8-mIgG1 (10 mg/kg) via intraperitoneal injection on days 5 and 12. Inflammatory responses were characterized at endpoint on day 16 by total and differential cell counts in bronchoalveolar lavage (BAL) fluid of the right lung. The left lung was fixed in 10% formalin for histopathology and mucus production assessment.

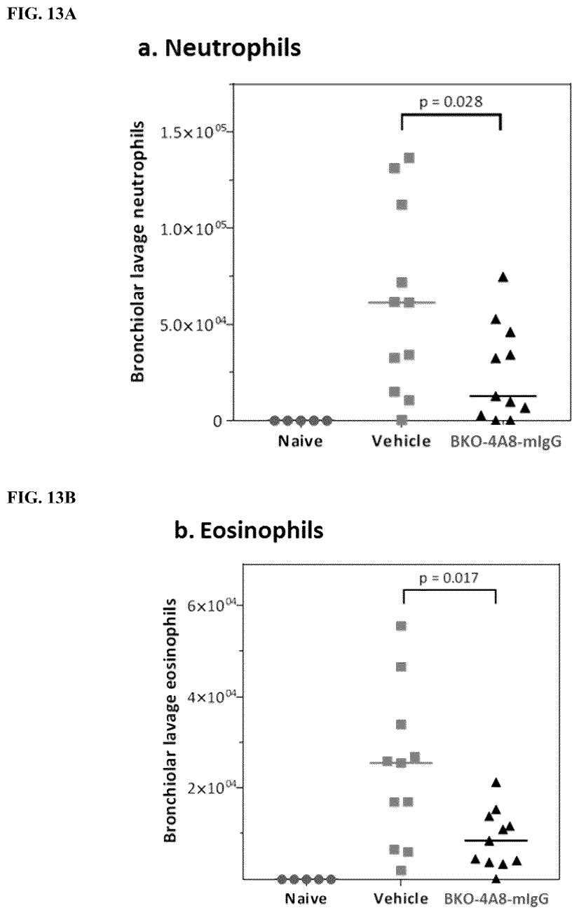

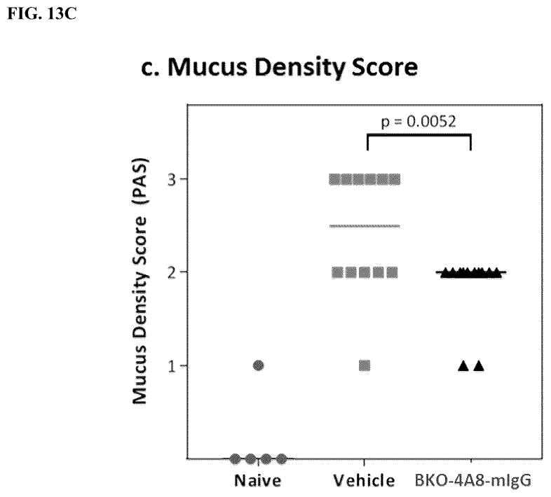

Anti-CXCR2 antibody BKO-4A8-mIgG1 treatment prior to HDM challenge resulted in a reduction in disease severity, including a significant (>60%) reduction in BAL eosinophil (p=0.017) and neutrophil (p=0.028) counts and reduced goblet cell hyperplasia (p=0.0052), when compared to the vehicle treated control group, as shown in FIGS. 13A, 13B, and 13C. Statistical evaluation was performed using a Mann-Whitney nonparametric unpaired t test with outliers identified using Grubbs' test (Alpha=0.01) to discern statistically significant differences between the vehicle and treatment groups. All statistical analyses were performed using GraphPad Prism™ 7.01.

The reduction in BAL eosinophil numbers in this model is surprising. The ability of BKO-4A8-mIgG1 to suppress eosinophilic migration supports its utility in treating diseases of eosinophilia, such as eosinophilic asthma, in addition to neutrophilic conditions.

### Example 11—LPS Induced Acute Lung Inflammation in Cynomolgus Monkey

Biologics naïve (i.e. not previously administered exogenous biologics) male cynomolgus monkeys were randomized into groups receiving vehicle or the anti-CXCR2 antibody BKO-4A8-101c administered by intravenous administration at a dose of 1 mg/kg on days 0, 14, and 28. One hour post-treatment of test antibodies on day 0, animals were exposed to aerosolized bacterial lipopolysaccharide (LPS) by inhalation of 20 μg/L for 5 mins (total dose 20 μg/kg). This is a well-established model of acute lung inflammation. Inflammatory responses were characterized at 24 hours after LPS exposure by total and differential cell counts of bronchoalveolar lavage fluid of the left lung and compared to matched counts from naïve animals at pre-treatment day 14. Blood was collected at various time points for differential cell count and serum collected for pharmacokinetic analysis.

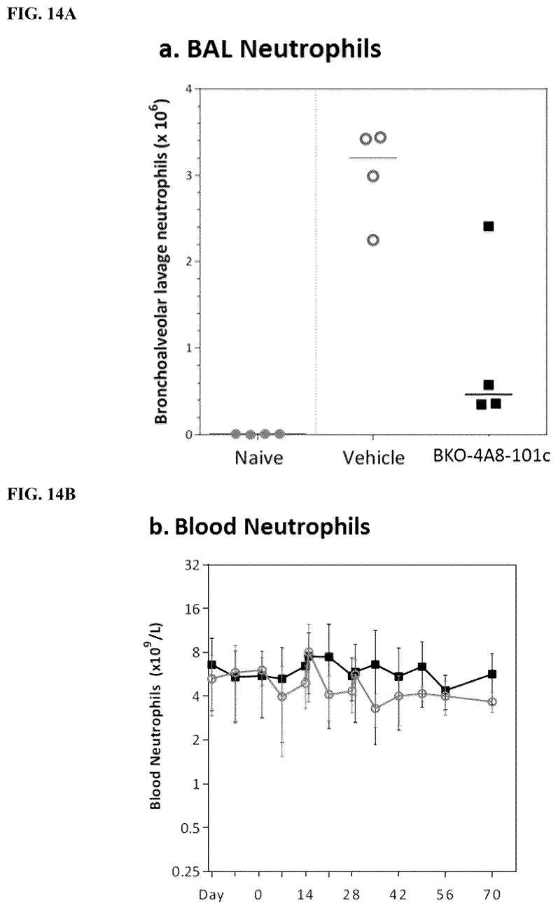

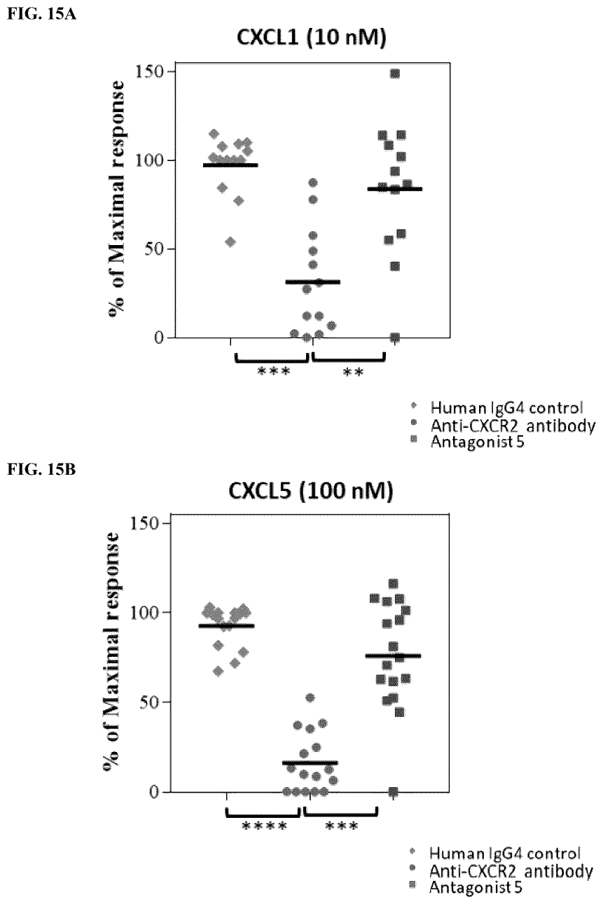

A single aerosol LPS treatment induced an influx of neutrophils into the lung in vehicle treated animals. Pre-treatment with 1 mg/kg of the anti-CXCR2 antibody BKO-4A8-101c markedly inhibited LPS-induced pulmonary neutrophilia in cynomolgus monkeys, as illustrated in FIG. 14A. Treatment was well tolerated with no loss in body weight. Treatment with BKO-4A8-101c did not induce measurable changes in blood neutrophil counts following repeat antibody dosing, as illustrated in FIG. 14B.

CXCR2 signaling is involved in neutrophil movement both out of the bone marrow and into peripheral tissues in response to chemokines produced by tissue-resident cells following stress, injury, or infection. CXCR2 binds multiple chemokines implicated in neutrophil recruitment and chronic inflammation, including CXCL1, CXCL5, and CXCL8. These chemokines are also elevated in patients with severe neutrophilic asthma and COPD. BKO-4A8-101c was shown herein to be a potent and specific antagonist of CXCR2-mediated signaling. Without wishing to be limited by any proposed mechanism of action, based on its in vitro profile, and its potency in the cynomolgus monkey, the anti-inflammatory activity of BKO-4A8-101c appears to be mediated via antagonism of CXCL1 and CXCL5-mediated CXCR2 signaling. These data support the concept that the CXCR2 receptor is the predominant chemokine receptor controlling neutrophil migration into the lungs under inflammatory conditions, and are consistent with the lack of marked efficacy of a humanized neutralizing anti-CXCL8 antibody administered to COPD patients (as discussed in MAHLER, D. A., et al., Efficacy and safety of a monoclonal antibody recognizing interleukin-8 in COPD: a pilot study. Chest, (2004) 126, 926-34), an approach that would only partially inhibit the CXCR1/CXCR2 inflammatory axis. A component of the anti-inflammatory activity of BKO-4A8-101c may be mediated via endothelial and epithelial cells, because CXCR2 expression on these cell types has also been implicated in neutrophil recruitment and lung injury (as discussed in REUTERSHAN, J., et al., Critical role of endothelial CXCR2 in LPS-induced neutrophil migration into the lung. J Clin Invest, (2006) 116, 695-702). The demonstrated in-vivo efficacy of BKO-4A8-101c in inhibiting lung neutrophil migration in response to LPS challenge without affecting circulating neutrophil numbers or any other measured safety parameters may be a consequence of its exquisite specificity and selective antagonist activity.

### Example 12—Anti-CXCR2 Antibody Efficiently Occupies the CXCR2 Receptor and Selectively Suppresses CXCR2-Mediated Neutrophil Responses

Receptor occupancy assays measure binding of a specific molecule or drug to a receptor expressed on a specific cell. This is a quantitative assay that can be used to evaluate receptor binding and other pharmacodynamic characteristics.

Receptor occupancy was examined in a human CXCR2 expressing cell line and human neutrophils enriched from whole blood. At least 85% of the CXCR2 receptors were occupied at 2 μg/mL of antibody. This amount was sufficient to suppress CXCL1-induced calcium flux by more than 85% in HTS002C—CHEMISCREEN™ human CXCR2 chemokine receptor calcium-optimized cells (results not shown).

While 2 μg/mL of antibody was sufficient to effectively antagonize CXCR2-mediated-signaling, it did not interfere with neutrophil functions. End-target chemoattractants C5a and fMLF bind to C5a receptor (CD88) and FPR1, respectively. Both of these agents induce chemotaxis and the expression of CD11b, a widely accepted marker of neutrophil activation in response to infection and inflammation. CXCR2 antibody did not suppress neutrophil CD11b upregulation or chemotaxis in response to C5a and fMLF (results not shown).

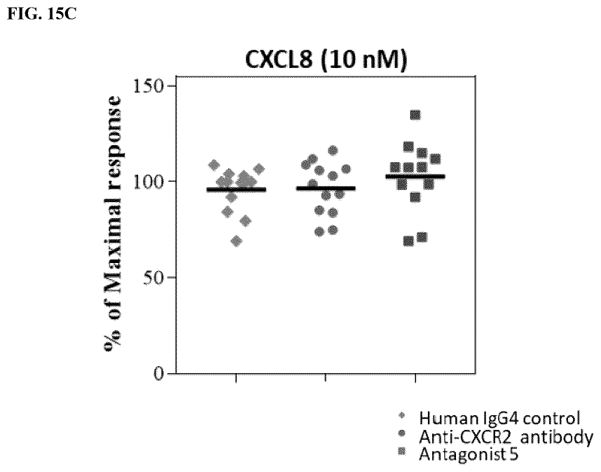

10 μg/ml (70 nM) CXCR2 antibody potently and specifically antagonized CXCR2-mediated responses to CXCL1 (p<0.0002, FIG. 15A) and CXCL5 (p<0.0001, FIG. 15B) in human neutrophils isolated (to 95%) from whole blood. 70 nM CXCR2 antibody demonstrated greater potency in this assay than the small molecule Antagonist 5 (danirixin) used at 1400 nM. This compares with previous reports that Agonist 5 inhibited CXCL1-induced ex vivo neutrophil surface expression of CD11b with an IC₅₀ of 69 ng/mL [156 nM], and IC₉₀ of 620 ng/ml (range 158-1080 ng/ml) [1400 nM, (range 356-2443 nM)] (Miller et al. “The pharmacokinetics and pharmacodynamics of danirixin (GSK1325756)—a selective CXCR2 antagonist—in healthy adult subjects” BMC Pharmacology and Toxicology 2015; 16).

Neither CXCR2 nor Antagonist 5 significantly impacted the neutrophil response to CXCL8 in this assay (FIG. 15C). CXCL8 binds to both CXCR1 and CXCR2.

These data demonstrate that CXCR2 antibody is a potent and selective inhibitor of CXCR2 on human neutrophils.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

TABLE 19
Sequences

| | | | |
|---|---|---|---|
| Sequence | | Sequence | |
| Identi- | | Identi- | |
| fier | Sequence | fier | Sequence |

BKO-1A1

| | | | |
|---|---|---|---|
| SEQ ID | QLQLQESGPGLVKPSETLSLTCTVSGGSIRTSS | SEQ ID | SSELTQDPAVSVALGQTVRITCQGDSLRYY |
| NO: 1 | YYWGWIRQPPGKGLEYIGSIYYSGTTYYNPS | NO: 2 | YASWYQQKPGQAPVLVIYDENSRPSGIPDR |
| (BKO_ | LKSRVTMSVDTSKNQFSLKMSSVTAADSAV | (BKO_ | 1 FSGSSSGNTASLSITGTQAEDEADYYCNSR |
| 1A1_VH) | YYCARHGRVREVPPFDYWGQGTLVTVSS | A1_VL) | DTSGNHWAFGGGTKLTVL |

BKO-1B10

| | | | |
|---|---|---|---|
| SEQ ID | QLQLQESGPGLVKPSETLSLTCTVSGGSIRTSS | SEQ ID | SSELTQDPAVSVALGQTVRITCQGDSLRYY |
| NO: 3 | YYWGWIRQPPGKGLEYIGSIYYSGTTYYNPS | NO: 4 | YASWYQQKPGQAPVLVIYDENSRPSGIPDR |
| (BKO_ | LKSRVTMSVDTSKNQFSLKLSSVTAADTAVY | (BKO_ | FSGSSSGNTASLRITGTQAEDEADYYCNSR |
| 1B10_VH) | YCARHGRVREVPPFDYWGQGTLVTVSS | 1B10_VL) | DTSGNHWAFGGGTKLTVL |

BKO-1D1

| | | | |
|---|---|---|---|
| SEQ ID | EVQLLESGGGLVQPGGSLRLSCAASKLTFKN | SEQ ID | QSALTQPPSASGSPGQSVTISCTGTSSDVGG |
| NO: 5 | SAMSWVRQAPGKGLEWVSAITGSGGRTYYA | NO: 6 | YNYVSWYQQHPGKAPKLMIYEVNKRPSGV |
| (BKO_ | DSVKGRFTISRDNSKNTLYLQMNSLRAEDTA | (BKO_ | PARFSGSKSGNTASLTVSGLQAEDEADYYC |
| 1D1_VH) | IYYCAIQMGYWGQGILVTVSS | 1D1_VL) | SSYAGSNNFGVFGGGTKLTVL |

BKO-1H3

| | | | |
|---|---|---|---|
| SEQ ID | EVQLLESGGGLVQPGGSLRLSCAASGFTLSRS | SEQ ID | QSALTQPASVSGSPGQSITISCTGTSSDVGG |
| NO: 7 | STSWVRQTPGKGLEWVSAISGSGGRTYYADS | NO: 8 | YNYVSWYQQHPGKAPKLMIYDVSNRPSGV |
| (BKO_ | VKGRFTISRDNSKNTLYLQMNSLRAEDTAVY | (BKO_ | SNRFSGSKSGNTASLTISGLQAEDEADYYC |
| 1H3_VH) | YCAIQLGYWGQGILVTVSS | 1H3_VL) | SSYTSSSTWVFGGGTKLTVL |

BKO-2D8

| | | | |
|---|---|---|---|
| SEQ ID | EVQLLESGGGLVQPGGSLRLSCAASGYTFTSS | SEQ ID | QSALTQPPSASGSPGQSVTISCTGTSSDIGG |
| NO: 9 | TMSWVRQAPGKGLEWVTAISGRGGRTYYAD | NO: 10 | YNYVSWYQQHPGKAPKLVIYEVNMRPSGV |
| (BKO_ | SVKGRFTISRDNSKNTLYLQMNSLRAEDTAV | (BKO_ | PARFSGSKSGNTASLTVSGLQAEDEADYYC |
| 2D8_VH) | YYCAIQLGNWGQGILVTVSS | 2D8_VL) | SSYAGNDNFGVFGGGTKLSVL |

BKO-3A9_b

| | | | |
|---|---|---|---|
| SEQ ID | QVQVQQSGPGLVKPSQTLSLTCAISGDSVSSN | SEQ ID | QSALTQPASASGSPGQSITISCTGTSSDVGN |
| NO: 11 | SAAWNWIRQSPSRGLEWLGRTYYRSKWYND | NO: 12 | YNRVSWYQQHPGKVPKLMIYEGSKRPSGIS |
| (BKO_ | YAVSLKRRITIRPDTSRNHFSLHLSSVTPEDTA | (BKO_ | NRFSGSKSGNTASLTISGLQPEDEADYFCCS |
| 3A9_VH) | VYYCVRAYCGGGSCLDYWGQGTLVTVSS | 3A9_L3_ | YAGSNTLVFGGGTKLTVL |
| | | E03_VL) | |

BKO-3D6

| | | | |
|---|---|---|---|
| SEQ ID | EVQLVESGGDLVQPGRSLRLSCAASGFTFDD | SEQ ID | SYELTQPPSVSVSPGQTASITCSGDKLGDKY |
| NO: 13 | YAMHWVRQAPGKGLKWVSGITWNSGNKRY | NO: 14 | ACWYQQKPGQSPVLVIYQDSKRPSGIPERF |
| (BKO_ | ADSVKGRFTISRDNAKNSLYLQMNSLRAEDT | (BKO_ | SGSNSGNTATLTISGTQAMDEADYYCQAW |
| 3D6_VH) | ALYYCAKDMKGSGTYFPAFDYWGQGTLVT | 3D6_L6_ | DSSTVVFGGGTKLTVL |
| | VSS | G06_VL | |
| | | (BKO_ | |
| | | 5H4_VL)) | |

BKO-3F4

| | | | |
|---|---|---|---|
| SEQ ID | EVQLLESGGGLVQPGGSLRLSCAASGLTFSSY | SEQ ID | QSALTQPPSASGSPGQSVTMSCTGTSSDVG |
| NO: 15 | AMSWVRQAPGKGLEWVSAISGSGGKIYYAD | NO: 16 | GYNYVTWYQQHPGKAPKLMIYEVSKRPSG |
| (BKO_ | SVKGRFTISRDNSKNTLYLQMNSLRAEDTAV | (BKO_ | VPARFSGSKSGNTASLTVSGLQAEDEADYY |
| 3F4_VH) | YYCAIQVGYWGQGTLVTVSS | 3F4_ | CSSYAGPNNFGVFGGGTKLTVL |
| | | L11_A | |
| | | 11_VL) | |

BKO-4A8

| | | | |
|---|---|---|---|
| SEQ ID | EVQLLESGGGLVQPGGSLRLSCAASGFTFSSS | SEQ ID | QSALTQPPSASGSPGQSVTISCIGTSSDVGG |
| NO: 17 | TMSWVRQAPGKGLEWVSAISGRGRNTYYAD | NO: 18 | YNYVSWYQQHPDKAPKLMIYEVNKRPSGV |
| (BKO_ | SVKGRFTISRDNSRNTLYLQMNSLRAEDTAV | (BKO_ | PDRFSGSKSGNTASLTVSGLQAEDEADYYC |
| 4A8_VH) | YYCAIQMGYWGQGILVTVSS | 4A8_VL) | SSYAGNNNFGVFGGGTKLTVL |

BKO-4F10

| | | | |
|---|---|---|---|
| SEQ ID | QVQLVQSGAEVKKPGASVKVSCKASGYTFT | SEQ ID | QSALTQPASVSGSPGQSITISCTGTSSDVGG |
| NO: 19 | GYYIHWVRQAPGQGLEWMGRFNPNNGGTN | NO: 20 | YNYVSWYQQHPGKAPKLMIYDVSNRPSGI |
| (BKO_ | YAQRFQGRVTMTRDTSISTAYMELSRLRSDD | (BKO_ | SNRFSGSKSGNTASLTISGLQAEDEADYYC |
| 4F10_VH) | TAVYYCARGPTIRLWFDNWFDSWGQGTLVT | 4F10_VL) | SSYTSSSTWVFGGGTKLTVL |
| | VSS | | |

BKO-5E8

| | | | |
|---|---|---|---|
| SEQ ID | EVQLLESGGGLVQPGGSLRLSCAASGFTFSSY | SEQ ID | SYELTQPPSVSVSPGQTANITCSGDTLGDKF |
| NO: 21 | AMSWVRQAPGRGLEWVSAIRGSGAGTYYA | NO: 22 | ACWYQQKPGQSPVLVIYQDTKRPSGIPERF |
| (BKO_ | DSMKGRFTISRDNSKDTLYLLMNSLRAEDTA | (BKO_ | SGSKSGITATLTISGTQAMDEADFYCQAWN |
| 5E8_H5_ | VYYCSKLEAVSGTGKYFQHWGQGTLVTVSS | 5E8_L3_ | SRGVVFGGGTRLTVL |
| C05_VH) | | C03_VL) | |

BKO-5G11

| | | | |
|---|---|---|---|
| SEQ ID | EVQLLESGGGLVQPGGSLRLSCAVSGFTFSN | SEQ ID | QSALTQPPSASGSPGQSVTISCTGTSSDVGG |
| NO: 23 | YAMTWVRQAPGKGLEWVSAISGRGSRTYYA | NO: 24 | YNYVSWYQQHPGKAPKLMIFEVSKRPSGV |
| (BKO_ | DSVKGRFTISRDTSKNTLYLQMNSLRAEDTA | (BKO_ | PDRFSGSKSGNTASLTVSGLQAEDEADYYC |
| 5G11_VH) | VYYCAKMDYWGQGTLVTVSS | 5G11_VL) | SSYAGSNNFGVFGGGTKLTVL |

BKO-5G6_c

| | | | |
|---|---|---|---|
| SEQ ID | QVQLVQSGAEVTKPGASVKVSCKASGYTFT | SEQ ID | QSALTQPASVSGSPGQSITISCTGTSSDVGG |
| NO: 25 | GYYIHWVRQAPGQGLEWMGRFNPNNGGTN | NO: 26 | YNYVSWYQQHPGKAPKLMIYDVSNRPSGI |
| (BKO_ | YAQRFQGRVTMTRDTSISTAYMELSRLRSDD | (BKO_ | SNRFSGSKSGNTASLTISGLQAEDEADYYC |
| 5G6_VH) | TAVYYCARGPTIRLWFDNWFDSWGQGTLVT | 5G6_ | SSYTSSSTWVFGGGTTLTVL |
| | VSS | L12_E | |
| | | 12_VL) | |

BKO-6A1_b

| | | | |
|---|---|---|---|
| SEQ ID | QVQLKQWGAGLLKPSETLSLTCAVYGGSFSG | SEQ ID | SSELTQDPAVSVALGQTVRITCQGDSLRSY |
| NO: 27 | YYWTWIRQPPGKGLEWIGEINHSGSTNYNPS | NO: 28 | YASWYQQKPGQAPVLVIYGKNNRPSGIPD |
| (BKO_ | LKSRVTMSVDTSKNQFSLKLRSVTAADTAVY | (BKO_ | RFSGSSSGNTASLTITGAQAEDEADYYCNS |
| 6A1_ | YCARGEVRGLITLYWYFDVWGRGSLVTVSS | 6A1_ | RDSSGNHVVFGGGTKLTVL |
| H4_A04_VH) | | E10_VL) | |

BKO-6A2_a

| | | | |
|---|---|---|---|
| SEQ ID | QVQLVESGGGVVQPGRSLRLSCAASGFTFST | SEQ ID | SYELTQPPSVSVSPGQTASITCSGDKLGDKY |
| NO: 29 | YDIHWVRQAPGKGLEWVAVIWYDGSNKYY | NO: 30 | ACWYQQKPGQSPVLVIYQDSKRPSGIPERF |
| (BKO_ | ADSVKGRFTISRDNSKNTLYLQMNSLRAEDT | (BKO_ | SGSNSGNTATLTISGTQAMDEADYYCQAW |
| 6A2_ | AVYYCARDEGYNYGYGGYWGQGTLVTVSS | 6A2_L6_ | DSSTVVFGGGTKLTVL |
| H1_B | | A06_VL) | |
| 01_VH) | | | |

BKO-7C11

| | | | |
|---|---|---|---|
| SEQ ID | QVQLQQWGAGLLKPSETLSLTCAVYGGSFSG | SEQ ID | SSELTQGPAVSVALGQTVRITCQGNSLRFY |
| NO: 31 | YYWSWIRQPPGKGLEWIGEINHSRNTNYNPS | NO: 32 | YASWYQQRPGQAPILVIYDKNNRPSGIPDR |
| (BKO_ | LKSRVTISVDTSKNQFSLKLSSVTAADTAVY | (BKO_ | FSGSSSGNTASLTITGAQAEDEADYYCNSR |
| 7C11_ | YCARGEVRGVFTLYWYFDVWGRGTLVTVSS | 7C11_ | DSSGYYMIFGGGTKLTVL |
| H6_B | | G01_ | |
| 06_VH) | | VL) | |

BKO-7G10_a

| | | | |
|---|---|---|---|
| SEQ ID | EVQLLESGGGLVQPGGSLRLSCAVSGFTFSN | SEQ ID | QSALTQPPSASGSPGQSVTISCTGTSSDVGG |
| NO: 33 | YAMTWVRQAPGKGLEWVSAISGRGSRTYYA | NO: 34 | YNYVSWYQQHPGKAPKLMIFEVSKRPSGV |
| (BKO_ | DSVKGRFTISRDTSKNTLYLQMNSLRAEDTA | (BKO_ | PDRFSGSKSGNTASLTVSGLQAEDEADYYC |
| 7G10_ | VYYCAKMDYWGQGTLVTVSS | 7G10_ | SSYAGSNNFGVFGGGTKLTVL |
| H1_ | | L6_E | |
| B01_VH) | | 06_VL) | |

BKO-7H8_b

| | | | |
|---|---|---|---|
| SEQ ID | EVQLLESGGGLVQPGGSLRLSCAASGYTFTSS | SEQ ID | QSALTQPPSASGSPGQSVTISCTGTSSDIGG |
| NO: 35 | TMSWVRQAPGKGLEWVTAISGRGGRTYYAD | NO: 36 | YNYVSWYQQHPGKAPKLVIYEVNMRPSGV |
| (BKO_ | SVKGRFTISRDNSKNTLYLQMNSLRAEDTAV | (BKO_ | PARFSGSKSGNTASLTVSGLQAEDEADYYC |
| 7H8_ | YYCAIQLGNWGQGILVTVSS | 7H8_ | SSYAGNDNFGVFGGGTKLSVL |
| H3_C | | L10_F | |
| 03_VH) | | 10_VL) | |

BKO-8B6

| | | | |
|---|---|---|---|
| SEQ ID | EVQLLESGGGLVQPGGSLRLSCAASGFTFSSN | SEQ ID | QSALTQPPSASGSPGQSVTISCTGTSSDVGG |
| NO: 37 | AMSWVRQAPGKGLEWVSAISNSGRSTYYAD | NO: 38 | YNYVSWYQQHPGKAPKLMMYEVSKRPSG |
| (BKO_ | SVKGRFTISRDSSKNTLYLLMNSLRAEDSAV | (BKO_ | VPDRFSGSKSGNTASLTVSGLQAEDEADYY |
| 8B6_VH) | YYCAIKLGYWGQGSLVTVSS | 8B6_VL) | CSSYAGSDNFGVFGGGTRLTVL |

BKO-8C4

| | | | |
|---|---|---|---|
| SEQ ID | QLQLQESGPGLVKPSETLSLTCTVSGGSIRTSS | SEQ ID | SSELTQDPAVSVALGQTVRITCQGDSLRYY |
| NO: 39 | YYWGWIRQPPGKGLEYIGSIYYSGTTYYNPS | NO: 40 | YASWYQQKPGQAPVLVIYDENSRPSGIPDR |
| (BKO_ | LKSRVTMSVDTSKNQFSLKLSSVTAADTAVY | (BKO_ | FSGSSSGNTASLRITGTQAEDEADYYCNSR |
| 8C4_VH) | YCARHGRVREVPPFDYWGQGTLVTVSS | 8C4_VL) | DTSGNHWAFGGGTKLTVL |

BKO-8G3_b

| | | | |
|---|---|---|---|
| SEQ ID | EVQLLESGGGLVQPGGSLRLSCAASGFTFSSY | SEQ ID | QSALTQPPSASGSPGQSVTISCTGTSSDVGG |
| NO: 41 | AMSWVRQAPGKGLEWVSAITGSGGSTYYAD | NO: 42 | YNYVSWYQQHPGKVPKLVIYEVSKRPSGV |
| (BKO_ | SVKGRFTISRDKSKNTLYLQMNSLRAEDTAV | (BKO_ | PDRFSGSKSGNTASLTVSGLQAEDEADYYC |
| 8G3_ | YYCAIRLGYWGQGSLVTVSS | 8G3_ | SSYAGSNNFGVFGGGTKLTVL |
| H4_D | | L1_G0 | |
| 04_VH) | | 1_VL) | |

BKO-8H10

| | | | |
|---|---|---|---|
| SEQ ID | QVQLVQSGAEVKKPGASVKVSCKASGYTFT | SEQ ID | QSALTQPPSASGSPGQSVTISFTGTSRDVGD |
| NO: 43 | GYYIHWVRQAPGQGLEWMGRIKPDSGGTNY | NO: 44 | YNYVSWYQQHPGKAPKLMIYEVNKRPSGV |
| (BKO_ | AQKFQGRVTMTRDTSITTAYMELSRLRSDDT | (BKO_ | PDRFSGSKSGNTASLTVSGLQAEDEADYYC |
| 8H10_VH) | AVYYCARGGSGWDYWGQGTLVTVSS | 8H10_VL) | SSYAGSNTYVFGTGTKVTVL |

BKO-8H8_b

| | | | |
|---|---|---|---|
| SEQ ID | EVQLLESGGGLVQPGGSLRLSCAASGLTVSS | SEQ ID | QSALTQPPSASGSPGQSVTMSCTGTSSDVG |
| NO: 45 | YAMSWVRQAPGKGLEWVSAISGSGGKIYYA | NO: 46 | GYNYVTWYQQHPGKAPKLVIYEVSKRPSG |
| (BKO_ | DSVKGRFTISRDNSKNTLYLQMNSLSAEDTA | (BKO_ | VPVRFSGSKSGNTASLTVSGLQAEDEADYY |
| 8H8_ | VYYCAIQVGYWGQGTLVTVSS | 8H8_ | CSSYAGPNNFGIFGGGTKLTVL |
| H5_E0 | | L7_H0 | |
| 5_VH) | | 8_VL) | |

BKO-9A8

| | | | |
|---|---|---|---|
| SEQ ID | EVQLLESGGGLVQTGGSLRLSCAASGFTFSSN | SEQ ID | QSALTQPPSASGSPGQSVTISCTGTSSDVGA |
| NO: 47 | TMSWVRQAPGKGLEWVSAISGSGGRTYYVD | NO: 48 | YNYVSWYQQHPGKAPKLMIYEVTKRPSGV |
| (BKO_ | SVKGRFTISRDNSKNTLYLQMHSLRAEDTAV | (BKO_ | PDRFSGSKSGNTASLTVSGLQAEDEADYYC |
| 9A8_ | YYCAIQLGSWGQGILVTVSS | 9A8_ | SSYAGNNNFGVFGGGTKLTVL |
| H3_F0 | | L1_H0 | |
| 3_VH) | | 2_VL) | |

BKO-9C3_a

| | | | |
|---|---|---|---|
| SEQ ID | EVQVLESGGGLVQPGGSLRLSCAASGFTFRS | SEQ ID | QSALTQPPSASGSPGQSVIISCTGTSSDVGG |
| NO: 49 | YAMSWVRQAPGKGLEWVSAISGSGGSTYYA | NO: 50 | YNYVSWYQQHPGKVPKLMIYEVSKRPSGV |
| (BKO_ | DSVKGRFTISRDKSKNTLYLQMNSLRAEDTA | (BKO_ | PDRFSGSKSGNTASLTVSGLQAEDEADYYC |
| 9C3_ | VYYCAIQLGYWGQGTLVTVSS | 9C3_ | TSYAGSNNFGVFGGGTKLTVL |
| H8_G | | L1_F0 | |
| 08_VH) | | 1_VL) | |

BKO-10G10

| | | | |
|---|---|---|---|
| SEQ ID | QLQLQESGPGLVKPSETLSLTCTVSGGSIRRSS | SEQ ID | QSALTQPASVSGSPGQSITISCTGTSSDVGG |
| NO: 51 | YYWGWIRQPPGKGLEWIGSFYNSGNTYYKP | NO: 52 | YNYVSWCQQHPGKAPKIMIFDVSNRPSGVS |
| (BKO_ | SLKSRVAISVDTPKNQFSLKLSSVTAADTAVY | (BKO_ | NRFSGSKSGNTASLTISGLQAEDEADYYCS |
| 10G10_ | YCARGYSSGGFDPWGQGTLVTVSS | 10G10_ | SYTSSSTWVFGGGTRLTVL |
| VH) | | VL) | |

BKO-4A8 single aa variants

| | | | |
|---|---|---|---|
| SEQ ID | EVQLLESGGGLVQPGGSLRLSCAASGFTFSSQ | SEQ ID | EVQLLESGGGLVQPGGSLRLSCAASGFTFS |
| NO: 53 | TMSWVRQAPGKGLEWVSAISGRGRNTYYAD | NO: 54 | SHTMSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQMGYWGQGILVTVSS | VH | DTAVYYCAIQMGYWGQGILVTVSS |
| S32Q) | | S32H) | |
| SEQ ID | EVQLLESGGGLVQPGGSLRLSCAASGFTFSSL | SEQ ID | EVQLLESGGGLVQPGGSLRLSCAASGFTFS |
| NO: 55 | TMSWVRQAPGKGLEWVSAISGRGRNTYYAD | NO: 56 | SWTMSWVRQAPGKGLEWVSAISGRGRNT |
| (4A8 | SVKGRFTISRDNSRNTLYLQMNSLRAEDTAV | (4A8 | YYADSVKGRFTISRDNSRNTLYLQMNSLRA |
| VH | YYCAIQMGYWGQGILVTVSS | VH | EDTAVYYCAIQMGYWGQGILVTVSS |
| S32L) | | S32W) | |
| SEQ ID | EVQLLESGGGLVQPGGSLRLSCAASGFTFSSY | SEQ ID | EVQLLESGGGLVQPGGSLRLSCAASGFTFS |
| NO: 57 | TMSWVRQAPGKGLEWVSAISGRGRNTYYAD | NO: 58 | SSAMSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQMGYWGQGILVTVSS | VH | DTAVYYCAIQMGYWGQGILVTVSS |
| S32Y) | | T33A) | |
| SEQ ID | EVQLLESGGGLVQPGGSLRLSCAASGFTFSSS | SEQ ID | EVQLLESGGGLVQPGGSLRLSCAASGFTFS |
| NO: 59 | TQSWVRQAPGKGLEWVSAISGRGRNTYYAD | NO: 60 | SSTDSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQMGYWGQGILVTVSS | VH | DTAVYYCAIQMGYWGQGILVTVSS |
| M34Q) | | M34D) | |
| SEQ ID | EVQLLESGGGLVQPGGSLRLSCAASGFTFSSS | SEQ ID | EVQLLESGGGLVQPGGSLRLSCAASGFTFS |
| NO: 61 | THSWVRQAPGKGLEWVSAISGRGRNTYYAD | NO: 62 | SSTWSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQMGYWGQGILVTVSS | VH | DTAVYYCAIQMGYWGQGILVTVSS |
| M34H) | | M34W) | |
| SEQ ID | EVQLLESGGGLVQPGGSLRLSCAASGFTFSSS | SEQ ID | EVQLLESGGGLVQPGGSLRLSCAASGFTFS |
| NO: 63 | TMSWVRQAPGKGLEWVSAHSGRGRNTYYA | NO: 64 | SSTMSWVRQAPGKGLEWVSAISDRGRNTY |
| (4A8 | DSVKGRFTISRDNSRNTLYLQMNSLRAEDTA | (4A8 | YADSVKGRFTISRDNSRNTLYLQMNSLRAE |
| VH | VYYCAIQMGYWGQGILVTVSS | VH | DTAVYYCAIQMGYWGQGILVTVSS |
| I51H) | | G52aD) | |
| SEQ ID | EVQLLESGGGLVQPGGSLRLSCAASGFTFSSS | SEQ ID | EVQLLESGGGLVQPGGSLRLSCAASGFTFS |
| NO: 65 | TMSWVRQAPGKGLEWVSAISGSGRNTYYAD | NO: 66 | SSTMSWVRQAPGKGLEWVSAISGQGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQMGYWGQGILVTVSS | VH | DTAVYYCAIQMGYWGQGILVTVSS |
| R53S) | | R53Q) | |
| SEQ ID | EVQLLESGGGLVQPGGSLRLSCAASGFTFSSS | SEQ ID | EVQLLESGGGLVQPGGSLRLSCAASGFTFS |
| NO: 67 | TMSWVRQAPGKGLEWVSAISGRDRNTYYAD | NO: 68 | SSTMSWVRQAPGKGLEWVSAISGRGRSTY |
| (4A8 | SVKGRFTISRDNSRNTLYLQMNSLRAEDTAV | (4A8 | YADSVKGRFTISRDNSRNTLYLQMNSLRAE |
| VH | YYCAIQMGYWGQGILVTVSS | VH | DTAVYYCAIQMGYWGQGILVTVSS |
| G54D) | | N56S) | |
| SEQ ID | EVQLLESGGGLVQPGGSLRLSCAASGFTFSSS | SEQ ID | EVQLLESGGGLVQPGGSLRLSCAASGFTFS |
| NO: 69 | TMSWVRQAPGKGLEWVSAISGRGRNTYYAD | NO: 70 | SSTMSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAKQMGYWGQGILVTVSS | VH | DTAVYYCAIQAGYWGQGILVTVSS |
| I94K) | | M96A) | |
| SEQ ID | EVQLLESGGGLVQPGGSLRLSCAASGFTFSSS | SEQ ID | EVQLLESGGGLVQPGGSLRLSCAASGFTFS |
| NO: 71 | TMSWVRQAPGKGLEWVSAISGRGRNTYYAD | NO: 72 | SSTMSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQQGYWGQGILVTVSS | VH | DTAVYYCAIQKGYWGQGILVTVSS |
| M96Q) | | M96K) | |
| SEQ ID | EVQLLESGGGLVQPGGSLRLSCAASGFTFSSS | SEQ ID | EVQLLESGGGLVQPGGSLRLSCAASGFTFS |
| NO: 73 | TMSWVRQAPGKGLEWVSAISGRGRNTYYAD | NO: 74 | SSTMSWVRQAPGKGLEWVSAISGRGRNTY |
| (4A8 | SVKGRFTISRDNSRNTLYLQMNSLRAEDTAV | (4A8 | YADSVKGRFTISRDNSRNTLYLQMNSLRAE |
| VH | YYCAIQMDYWGQGILVTVSS | VH | DTAVYYCAIQMGSWGQGILVTVSS |
| G101D) | | Y102S) | |
| SEQ ID | EVQLLESGGGLVQPGGSLRLSCAASGFTFSSS | SEQ ID | QSALTQPPSASGSPGQSVTISCIGTSSDVGG |
| NO: 75 | TMSWVRQAPGKGLEWVSAISGRGRNTYYAD | NO: 76 | YNYVSWYQQHPDKAPKLMIYDVNKRPSG |
| (4A8 | SVKGRFTISRDNSRNTLYLQMNSLRAEDTAV | (4A8 | VPDRFSGSKSGNTASLTVSGLQAEDEADYY |
| VH | YYCAIQMGKWGQGILVTVSS | VL | CSSYAGNNNFGVFGGGTKLTVL |
| Y102K) | | E50D) | |
| SEQ ID | QSALTQPPSASGSPGQSVTISCIGTSSDVGGY | SEQ ID | QSALTQPPSASGSPGQSVTISCIGTSSDVGG |
| NO: 77 | NYVSWYQQHPDKAPKLMIYEVDKRPSGVPD | NO: 78 | YNYVSWYQQHPDKAPKLMIYEVSKRPSGV |
| (4A8 | RFSGSKSGNTASLTVSGLQAEDEADYYCSSY | (4A8 | PDRFSGSKSGNTASLTVSGLQAEDEADYYC |
| VL | AGNNNFGVFGGGTKLTVL | VL | SSYAGNNNFGVFGGGTKLTVL |
| N52D) | | N52S) | |
| SEQ ID | QSALTQPPSASGSPGQSVTISCIGTSSDVGGY | SEQ ID | QSALTQPPSASGSPGQSVTISCIGTSSDVGG |
| NO: 79 | NYVSWYQQHPDKAPKLMIYEVNARPSGVPD | NO: 80 | YNYVSWYQQHPDKAPKLMIYEVNDRPSGV |
| (4A8 | RFSGSKSGNTASLTVSGLQAEDEADYYCSSY | (4A8 | PDRFSGSKSGNTASLTVSGLQAEDEADYYC |
| VL | AGNNNFGVFGGGTKLTVL | VL | SSYAGNNNFGVFGGGTKLTVL |
| K53A) | | K53D) | |
| SEQ ID | QSALTQPPSASGSPGQSVTISCIGTSSDVGGY | SEQ ID | QSALTQPPSASGSPGQSVTISCIGTSSDVGG |
| NO: 81 | NYVSWYQQHPDKAPKLMIYEVNHRPSGVPD | NO: 82 | YNYVSWYQQHPDKAPKLMIYEVNKQPSG |
| (4A8 | RFSGSKSGNTASLTVSGLQAEDEADYYCSSY | (4A8 | VPDRFSGSKSGNTASLTVSGLQAEDEADYY |
| VL | AGNNNFGVFGGGTKLTVL | VL | CSSYAGNNNFGVFGGGTKLTVL |
| K53H) | | R54Q) | |
| SEQ ID | QSALTQPPSASGSPGQSVTISCIGTSSDVGGY | SEQ ID | QSALTQPPSASGSPGQSVTISCIGTSSDVGG |
| NO: 83 | NYVSWYQQHPDKAPKLMIYEVNKRPSGVPD | NO: 84 | YNYVSWYQQHPDKAPKLMIYEVNKRPSGV |
| (4A8 | RFSGSKSGNTASLTVSGLQAEDEADYYCSSA | (4A8 | PDRFSGSKSGNTASLTVSGLQAEDEADYYC |
| VL | AGNNNFGVFGGGTKLTVL | VL | SSYAGANNFGVFGGGTKLTVL |
| Y91A) | | N94A) | |
| SEQ ID | QSALTQPPSASGSPGQSVTISCIGTSSDVGGY | SEQ ID | QSALTQPPSASGSPGQSVTISCIGTSSDVGG |
| NO: 85 | NYVSWYQQHPDKAPKLMIYEVNKRPSGVPD | NO: 86 | YNYVSWYQQHPDKAPKLMIYEVNKRPSGV |
| (4A8 | RFSGSKSGNTASLTVSGLQAEDEADYYCSSY | (4A8 | PDRFSGSKSGNTASLTVSGLQAEDEADYYC |
| VL | AGSNNFGVFGGGTKLTVL | VL | SSYAGKNNFGVFGGGTKLTVL |
| N945) | | N94K) | |
| SEQ ID | QSALTQPPSASGSPGQSVTISCIGTSSDVGGY | SEQ ID | QSALTQPPSASGSPGQSVTISCIGTSSDVGG |
| NO: 87 | NYVSWYQQHPDKAPKLMIYEVNKRPSGVPD | NO: 88 | YNYVSWYQQHPDKAPKLMIYEVNKRPSGV |
| (4A8 | RFSGSKSGNTASLTVSGLQAEDEADYYCSSY | (4A8 | PDRFSGSKSGNTASLTVSGLQAEDEADYYC |
| VL | AGLNNFGVFGGGTKLTVL | VL | SSYAGWNNFGVFGGGTKLTVL |
| N94L) | | N94W) | |
| SEQ ID | QSALTQPPSASGSPGQSVTISCIGTSSDVGGY | SEQ ID | QSALTQPPSASGSPGQSVTISCIGTSSDVGG |
| NO: 89 | NYVSWYQQHPDKAPKLMIYEVNKRPSGVPD | NO: 90 | YNYVSWYQQHPDKAPKLMIYEVNKRPSGV |
| (4A8 | RFSGSKSGNTASLTVSGLQAEDEADYYCSSY | (4A8 | PDRFSGSKSGNTASLTVSGLQAEDEADYYC |
| VL | AGYNNFGVFGGGTKLTVL | VL | SSYAGNNQFGVFGGGTKLTVL |
| N94Y) | | N95aQ) | |
| SEQ ID | QSALTQPPSASGSPGQSVTISCIGTSSDVGGY | SEQ ID | QSALTQPPSASGSPGQSVTISCIGTSSDVGG |
| NO: 91 | NYVSWYQQHPDKAPKLMIYEVNKRPSGVPD | NO: 92 | YNYVSWYQQHPDKAPKLMIYEVNKRPSGV |
| (4A8 | RFSGSKSGNTASLTVSGLQAEDEADYYCSSY | (4A8 | PDRFSGSKSGNTASLTVSGLQAEDEADYYC |
| VL | AGNNDFGVFGGGTKLTVL | VL | SSYAGNNHFGVFGGGTKLTVL |
| N95aD) | | N95aH) | |
| SEQ ID | QSALTQPPSASGSPGQSVTISCIGTSSDVGGY | SEQ ID | QSALTQPPSASGSPGQSVTISCIGTSSDVGG |
| NO: 93 | NYVSWYQQHPDKAPKLMIYEVNKRPSGVPD | NO: 94 | YNYVSWYQQHPDKAPKLMIYEVNKRPSGV |
| (4A8 | RFSGSKSGNTASLTVSGLQAEDEADYYCSSY | (4A8 | PDRFSGSKSGNTASLTVSGLQAEDEADYYC |
| VL | AGNNKFGVFGGGTKLTVL | VL | SSYAGNNLFGVFGGGTKLTVL |
| N95aK) | | N95aL) | |
| SEQ ID | QSALTQPPSASGSPGQSVTISCIGTSSDVGGY | SEQ ID | QSALTQPPSASGSPGQSVTISCIGTSSDVGG |
| NO: 95 | NYVSWYQQHPDKAPKLMIYEVNKRPSGVPD | NO: 96 | YNYVSWYQQHPDKAPKLMIYEVNKRPSGV |
| (4A8 | RFSGSKSGNTASLTVSGLQAEDEADYYCSSY | (4A8 | PDRFSGSKSGNTASLTVSGLQAEDEADYYC |
| VL | AGNNYFGVFGGGTKLTVL | VL | SSYAGNNNFGAFGGGTKLTVL |
| N95aY) | | V97A) | |
| SEQ ID | QSALTQPPSASGSPGQSVTISCIGTSSDVGGY | | |
| NO: 97 | NYVSWYQQHPDKAPKLMIYEVNKRPSGVPD | | |
| (4A8 | RFSGSKSGNTASLTVSGLQAEDEADYYCSSY | | |
| VL | AGNNNFGKFGGGTKLTVL | | |
| V97K) | | | |

BKO-4A8 combinatorial variants

| | | | |
|---|---|---|---|
| SEQ ID | EVQLLESGGGLVQPGGSLRLSCAASGFTFSSS | SEQ ID | EVQLLESGGGLVQPGGSLRLSCAASGFTFS |
| NO: 98 | TQSWVRQAPGKGLEWVSAISGRGRSTYYAD | NO: 99 | SSTQSWVRQPPGKGLEWVSAISGRGRSTYY |
| (4A8 VH | SVKGRFTISRDNSRNTLYLQMNSLRAEDTAV | (4A8 VH | ADSVKGRFTISRDNSRNTLYLQMNSLRAED |
| Variant | YYCAIQMGYWGQGILVTVSS | Variant | TAVYYCAIQMGYWGQGILVTVSS |
| 1 | | 2 | |
| (M34Q_ | | (M34Q_ | |
| N56S)) | | A40P_ | |
| | | N56S)) | |
| SEQ ID | EVQLLESGGGLVQPGGSLRLSCAASGFTFSSS | SEQ ID | EVQLLESGGGLVQPGGSLRLSCAASGFTFS |
| NO: 100 | TQSWVRQPPGKGLEWVSAISGRGRSTYYADS | NO: 101 | SSTQSWVRQPPGKGLEWVSAISGRGRSTYY |
| (4A8 VH | VKGRFTISRDNSKNTLYLQMNSLRAEDTAVY | (4A8 VH | ADSVKGRFTISRDNSRNTLYLQMNSLRAED |
| Variant | YCAIQMGYWGQGILVTVSS | Variant | TAVYYCAIQKGYWGQGILVTVSS |
| 3 | | 4 | |
| (M34Q_ | | (M34Q_ | |
| A40P_ | | A40P_ | |
| N56S_ | | N56S_ | |
| R75K)) | | M96K)) | |
| SEQ ID | EVQLLESGGGLVQPGGSLRLSCAASGFTFSSS | SEQ ID | EVQLLESGGGLVQPGGSLRLSCAASGFTFS |
| NO: 102 | TQSWVRQPPGKGLEWVSAISGRGRSTYYADS | NO: 103 | SSTQSWVRQPPGKGLEWVSAISGRGRSTYY |
| (4A8 VH | VKGRFTISRDNSKNTLYLQMNSLRAEDTAVY | (4A8 VH | ADSVKGRFTISRDNSKNTLYLQMNSLRAED |
| Variant | YCAIQKGYWGQGILVTVSS | Variant | TAVYYCAKQKGYWGQGILVTVSS |
| 5 | | 6 | |
| (M34Q_ | | (M34Q_ | |
| A40P_ | | A40P_ | |
| N56S_ | | N56S_ | |
| R75K_ | | R75K_ | |
| M96K)) | | I94K_ | |
| | | M96K)) | |
| SEQ ID | EVQLLESGGGLVQPGGSLRLSCAASGFTFSSS | SEQ ID | EVQLLESGGGLVQPGGSLRLSCAASGFTFS |
| NO: 104 | TQSWVRQPPGKGLEWVSAISGRGRSTYYADS | NO: 105 | SSTQSWVRQAPGKGLEWVSAISGRGRSTY |
| (4A8 VH | VKGRFTISRDNSRNTLYLQMNSLRAEDTAVY | (4A8 VH | YADSVKGRFTISRDNSRNTLYLQMNSLRAE |
| Variant | YCAKQKGYWGQGILVTVSS | Variant | DTAVYYCAIQKGYWGQGILVTVSS |
| 7 | | 8 | |
| (M34Q_ | | (M34Q_ | |
| A40P_ | | N56S_ | |
| N56S_ | | M96K)) | |
| 194K_ | | | |
| M96K)) | | | |
| SEQ ID | EVQLLESGGGLVQPGGSLRLSCAASGFTFSSS | SEQ ID | EVQLLESGGGLVQPGGSLRLSCAASGFTFS |
| NO: 106 | TQSWVRQAPGKGLEWVSAISGRGRSTYYAD | NO: 107 | SSTMSWVRQAPGKGLEWVSAISGRGRNTY |
| (4A8 VH | SVKGRFTISRDNSKNTLYLQMNSLRAEDTAV | (4A8 VH | YADSVKGRFTISRDNSRNTLYLQMNSLRAE |
| Variant | YYCAIQKGYWGQGILVTVSS | Variant | DTAVYYCAKQKGYWGQGILVTVSS |
| 9 | | 10 | |
| (M34Q_ | | I94K_ | |
| N56S_ | | M96K) | |
| R75K_ | | | |
| M96K)) | | | |
| SEQ ID | EVQLLESGGGLVQPGGSLRLSCAASGFTFSSS | SEQ ID | QSALTQPPSASGSPGQSVTISCIGTSSDVGG |
| NO: 108 | TQSWVRQPPGKGLEWVSAISGRGRSTYYADS | NO: 109 | YNYVSWYQQHPDKAPKLMIYEVSKRPSGV |
| (4A8 VH | VKGRFTISRDNSKNTLYLQMNSLRAEDTAVY | (4A8 VL | PDRFSGSKSGNTASLTVSGLQAEDEADYYC |
| Variant | YCAIQAGYWGQGILVTVSS | variant | SSYAGSNNFGVFGGGTKLTVL |
| 101 | | b | |
| M34Q_ | | N52S_ | |
| A40P_ | | N94S) | |
| N56S_ | | | |
| R75K_ | | | |
| M96A) | | | |
| SEQ ID | QSALTQPPSASGSPGQSVTISCIGTSSDVGGY | SEQ ID | GAAGTTCAGCTGCTTGAATCTGGCGGAG |
| NO: 110 | NYVSWYQQHPGKAPKLMIYEVSKRPSGVPD | NO: 111 | GACTGGTTCAGCCTGGCGGATCTCTGAGA |
| (4A8 VL | RFSGSKSGNTASLTVSGLQAEDEADYYCSSY | (4A8 VH | CTGTCTTGTGCCGCCAGCGGCTTCACCTT |
| variant | AGSNNFGVFGGGTKLTVL | M34Q_ | TAGCAGCAGCACACAGAGCTGGGTCCGA |
| c | | A40P_ | CAGCCTCCTGGCAAAGGACTGGAATGGG |
| D41G_ | | N56S_ | TGTCCGCCATCTCTGGCAGAGGCAGAAG |
| N52S_ | | R75K_ | CACCTACTACGCCGACTCTGTGAAGGGCA |
| N94S) | | M96A) | GATTCACCATCAGCCGGGACAACAGCAA |
| | | | GAACACCCTGTACCTGCAGATGAACAGC |
| | | | CTGAGAGCCGAGGACACCGCCGTGTACT |
| | | | ATTGTGCCATCCAGGCCGGCTATTGGGGC |
| | | | CAGGGAATACTCGTGACAGTGTCCTCA |
| SEQ ID | CAGTCTGCTCTGACACAGCCTCCTAGCGCC | | |
| NO: 112 | TCTGGCTCTCCTGGCCAGAGCGTGACCATC | | |
| (4A8 VL | AGCTGTATCGGCACCAGCAGCGACGTGGG | | |
| D41G_ | CGGCTACAACTACGTGTCCTGGTATCAGCA | | |
| N52S_ | GCACCCCGgTAAGGCCCCCAAGCTGATGAT | | |
| N94S) | CTACGAAGTGTCCAAGCGGCCCAGCGGCGT | | |
| | GCCCGATAGATTCAGCGGCAGCAAGAGCG | | |
| | GCAACACCGCCAGCCTCACAGTGTCTGGAC | | |
| | TGCAGGCCGAGGACGAGGCCGACTACTAC | | |
| | TGTAGCAGCTACGCCGGCAgCAACAACTTC | | |
| | GGCGTGTTCGGCGGAGGCACCAAGCTGAC | | |
| | AGTCCTA | | |

BKO-4A8-mIgG1

| | | | |
|---|---|---|---|
| SEQ ID | EVQLLESGGGLVQPGGSLRLSCAASGFTFSSS | SEQ ID | QSALTQPPSASGSPGQSVTISCIGTSSDVGG |
| NO: 113 | TMSWVRQAPGKGLEWVSAISGRGRNTYYAD | NO: 114 | YNYVSWYQQHPDKAPKLMIYEVNKRPSGV |
| (BKO- | SVKGRFTISRDNSRNTLYLQMNSLRAEDTAV | (BKO- | PDRFSGSKSGNTASLTVSGLQAEDEADYYC |
| 4A8- | YYCAIQMGYWGQGILVTVSSAKTTPPSVYPL | 4A8- | SSYAGNNNFGVFGGGTKLTVLGQPKSSPSV |
| mIgG1 | APGSAAQTNSMVTLGCLVKGYFPEPVTVTW | mIgG1 | TLFPPSSEELETNKATLVCTITDFYPGVVTV |
| VH) | NSGSLSSGVHTFPAVLQSDLYTLSSSVTVPSS | VL) | DWKVDGTPVTQGMETTQPSKQSNNKYMA |
| | PRPSETVTCNVAHPASSTKVDKKIVPRDCGC | | SSYLTLTARAWERHSSYSCQVTHEGHTVE |
| | KPCICTVPEVSSVFIFPPKPKDVLTITLTPKVTC | | KSLSRADCS |
| | VVVAISKDDPEVQFSWFVDDVEVHTAQTQP | | |
| | REEQFNSTFRSVSELPIMHQDWLNGKEFKCR | | |
| | VNSAAFPAPIEKTISKTKGRPKAPQVYTIPPPK | | |
| | EQMAKDKVSLTCMITDFFPEDITVEWQWNG | | |
| | QPAENYKNTQPIMNTNGSYFVYSKLNVQKS | | |
| | NWEAGNTFTCSVLHEGLHNHHTEKSLSHSPG | | |

BKO-4A8 heavy chain variant

| | | | |
|---|---|---|---|
| SEQ ID | EVQLLESGGGLVQPGGSLRLSCAASGFTFSSS | SEQ ID | ASTKGPSVFPLAPCSRSTSESTAALGCLVKD |
| NO: 115 | TMSWVRQAPGKGLEWVSAISGRGRNTYYAD | NO: 116 | YFPEPVTVSWNSGALTSGVHTFPAVLQSSG |
| (BKO- | SVKGRFTISRDNSRNTLYLQMNSLRAEDTAV | (IgG4*) | LYSLSSVVTVPSSSLGTKTYTCNVDHKPSN |
| 4A8 | YYCAIQMGYWGQGILVTVSSASTKGPSVFPL | | TKVDKRVESKYGPPCPPCPAPEFLGGPSVFL |
| IgG4*) | APCSRSTSESTAALGCLVKDYFPEPVTVSWN | | FPPKPKDTLYITREPEVTCVVVDVSQEDPEV |
| | SGALTSGVHTFPAVLQSSGLYSLSSVVTVPSS | | QFNWYVDGVEVHNAKTKPREEQFNSTYRV |
| | SLGTKTYTCNVDHKPSNTKVDKRVESKYGPP | | VSVLTVLHQDWLNGKEYKCKVSNKGLPSS |
| | CPPCPAPEFLGGPSVFLFPPKPKDTLYITREPE | | IEKTISKAKGQPREPQVYTLPPSQEEMTKNQ |
| | VTCVVVDVSQEDPEVQFNWYVDGVEVHNA | | VSLTCLVKGFYPSDIAVEWESNGQPENNYK |
| | KTKPREEQFNSTYRVVSVLTVLHQDWLNGK | | TTPPVLDSDGSFFLYSRLTVDKSRWQEGNV |
| | EYKCKVSNKGLPSSIEKTISKAKGQPREPQVY | | FSCSVMHEALHNHYTQKSLSLSLG |
| | TLPPSQEEMTKNQVSLTCLVKGFYPSDIAVE | | |
| | WESNGQPENNYKTTPPVLDSDGSFFLYSRLT | | |
| | VDKSRWQEGNVFSCSVMHEALHNHYTQKSL | | |
| | SLSLG | | |
| SEQ ID | EVQLLESGGGLVQPGGSLRLSCAASGFTFSSS | SEQ ID | ASTKGPSVFPLAPCSRSTSESTAALGCLVKD |
| NO: 117 | TMSWVRQAPGKGLEWVSAISGRGRNTYYAD | NO: 118 | YFPEPVTVSWNSGALTSGVHTFPAVLQSSG |
| (BKO- | SVKGRFTISRDNSRNTLYLQMNSLRAEDTAV | (IgG4) | LYSLSSVVTVPSSSLGTKTYTCNVDHKPSN |
| 4A8 | YYCAIQMGYWGQGILVTVSSASTKGPSVFPL | | TKVDKRVESKYGPPCPPCPAPEFLGGPSVFL |
| IgG4) | APCSRSTSESTAALGCLVKDYFPEPVTVSWN | | FPPKPKDTLMISRTPEVTCVVVDVSQEDPE |
| | SGALTSGVHTFPAVLQSSGLYSLSSVVTVPSS | | VQFNWYVDGVEVHNAKTKPREEQFNSTYR |
| | SLGTKTYTCNVDHKPSNTKVDKRVESKYGPP | | VVSVLTVLHQDWLNGKEYKCKVSNKGLPS |
| | CPPCPAPEFLGGPSVFLFPPKPKDTLMISRTPE | | SIEKTISKAKGQPREPQVYTLPPSQEEMTKN |
| | VTCVVVDVSQEDPEVQFNWYVDGVEVHNA | | QVSLTCLVKGFYPSDIAVEWESNGQPENNY |
| | KTKPREEQFNSTYRVVSVLTVLHQDWLNGK | | KTTPPVLDSDGSFFLYSRLTVDKSRWQEGN |
| | EYKCKVSNKGLPSSIEKTISKAKGQPREPQVY | | VFSCSVMHEALHNHYTQKSLSLSLG |
| | TLPPSQEEMTKNQVSLTCLVKGFYPSDIAVE | | |
| | WESNGQPENNYKTTPPVLDSDGSFFLYSRLT | | |
| | VDKSRWQEGNVFSCSVMHEALHNHYTQKSL | | |
| | SLSLG | | |
| SEQ ID | EVQLLESGGGLVQPGGSLRLSCAASGFTFSSS | SEQ ID | ASTKGPSVFPLAPCSRSTSESTAALGCLVKD |
| NO: 119 | TMSWVRQAPGKGLEWVSAISGRGRNTYYAD | NO: 120 | YFPEPVTVSWNSGALTSGVHTFPAVLQSSG |
| (BKO- | SVKGRFTISRDNSRNTLYLQMNSLRAEDTAV | (IgG2*) | LYSLSSVVTVPSSNFGTQTYTCNVDHKPSN |
| 4A8 | YYCAIQMGYWGQGILVTVSSASTKGPSVFPL | | TKVDKTVERKCCVECPPCPAPPVAGPSVFL |
| IgG2*) | APCSRSTSESTAALGCLVKDYFPEPVTVSWN | | FPPKPKDTLMISRTPEVTCVVVDVSHEDPE |
| | SGALTSGVHTFPAVLQSSGLYSLSSVVTVPSS | | VQFNWYVDGVEVHNAKTKPREEQFNSTFR |
| | NFGTQTYTCNVDHKPSNTKVDKTVERKCCV | | VVSVLTVVHQDWLNGKEYKCKVSNKGLP |
| | ECPPCPAPPVAGPSVFLFPPKPKDTLMISRTPE | | SSIEKTISKTKGQPREPQVYTLPPSREEMTK |
| | VTCVVVDVSHEDPEVQFNWYVDGVEVHNA | | NQVSLTCLVKGFYPSDIAVEWESNGQPENN |
| | KTKPREEQFNSTFRVVSVLTVVHQDWLNGK | | YKTTPPMLDSDGSFFLYSKLTVDKSRWQQ |
| | EYKCKVSNKGLPSSIEKTISKTKGQPREPQVY | | GNVFSCSVMHEALHNHYTQKSLSLSPG |
| | TLPPSREEMTKNQVSLTCLVKGFYPSDIAVE | | |
| | WESNGQPENNYKTTPPMLDSDGSFFLYSKLT | | |
| | VDKSRWQQGNVFSCSVMHEALHNHYTQKSL | | |
| | SLSPG | | |
| SEQ ID | EVQLLESGGGLVQPGGSLRLSCAASGFTFSSS | SEQ ID | ASTKGPSVFPLAPSSKSTSGGTAALGCLVK |
| NO: 121 | TMSWVRQAPGKGLEWVSAISGRGRNTYYAD | NO: 122 | DYFPEPVTVSWNSGALTSGVHTFPAVLQSS |
| (BKO- | SVKGRFTISRDNSRNTLYLQMNSLRAEDTAV | (IgG1*) | GLYSLSSVVTVPSSSLGTQTYICNVNHKPSN |
| 4A8 | YYCAIQMGYWGQGILVTVSSASTKGPSVFPL | | TKVDKKVEPKSCDKTHTCPPCPAPELAGAP |
| IgG1*) | APSSKSTSGGTAALGCLVKDYFPEPVTVSWN | | SVFLFPPKPKDTLMISRTPEVTCVVVDVSHE |
| | SGALTSGVHTFPAVLQSSGLYSLSSVVTVPSS | | DPEVKFNWYVDGVEVHNAKTKPREEQYN |
| | SLGTQTYICNVNHKPSNTKVDKKVEPKSCDK | | STYRVVSVLTVLHQDWLNGKEYKCKVSN |
| | THTCPPCPAPELAGAPSVFLFPPKPKDTLMISR | | KALPAPIEKTISKAKGQPREPQVYTLPPSRD |
| | TPEVTCVVVDVSHEDPEVKFNWYVDGVEVH | | ELTKNQVSLTCLVKGFYPSDIAVEWESNGQ |
| | NAKTKPREEQYNSTYRVVSVLTVLHQDWLN | | PENNYKTTPPVLDSDGSFFLYSKLTVDKSR |
| | GKEYKCKVSNKALPAPIEKTISKAKGQPREPQ | | WQQGNVFSCSVMHEALHNHYTQKSLSLSP |
| | VYTLPPSRDELTKNQVSLTCLVKGFYPSDIAV | | G |
| | EWESNGQPENNYKTTPPVLDSDGSFFLYSKL | | |
| | TVDKSRWQQGNVFSCSVMHEALHNHYTQKS | | |
| | LSLSPG | | |
| SEQ ID | EVQLLESGGGLVQPGGSLRLSCAASGFTFSSS | SEQ ID | ASTKGPSVFPLAPSSKSTSGGTAALGCLVK |
| NO: 123 | TMSWVRQAPGKGLEWVSAISGRGRNTYYAD | NO: 124 | DYFPEPVTVSWNSGALTSGVHTFPAVLQSS |
| (BKO- | SVKGRFTISRDNSRNTLYLQMNSLRAEDTAV | (IgG1) | GLYSLSSVVTVPSSSLGTQTYICNVNHKPSN |
| 4A8 | YYCAIQMGYWGQGILVTVSSASTKGPSVFPL | | TKVDKKVEPKSCDKTHTCPPCPAPELLGGP |
| IgG1) | APSSKSTSGGTAALGCLVKDYFPEPVTVSWN | | SVFLFPPKPKDTLMISRTPEVTCVVVDVSHE |
| | SGALTSGVHTFPAVLQSSGLYSLSSVVTVPSS | | DPEVKFNWYVDGVEVHNAKTKPREEQYN |
| | SLGTQTYICNVNHKPSNTKVDKKVEPKSCDK | | STYRVVSVLTVLHQDWLNGKEYKCKVSN |
| | THTCPPCPAPELLGGPSVFLFPPKPKDTLMISR | | KALPAPIEKTISKAKGQPREPQVYTLPPSRD |
| | TPEVTCVVVDVSHEDPEVKFNWYVDGVEVH | | ELTKNQVSLTCLVKGFYPSDIAVEWESNGQ |
| | NAKTKPREEQYNSTYRVVSVLTVLHQDWLN | | PENNYKTTPPVLDSDGSFFLYSKLTVDKSR |
| | GKEYKCKVSNKALPAPIEKTISKAKGQPREPQ | | WQQGNVFSCSVMHEALHNHYTQKSLSLSP |
| | VYTLPPSRDELTKNQVSLTCLVKGFYPSDIAV | | G |
| | EWESNGQPENNYKTTPPVLDSDGSFFLYSKL | | |
| | TVDKSRWQQGNVFSCSVMHEALHNHYTQKS | | |
| | LSLSPG | | |

CXCR Sequences

| | | | |
|---|---|---|---|
| SEQ ID | MEDFNMESDSFEDFWKGEDLSNYSYSSTLPP | SEQ ID | MGEFKVDKFNIEDFFSGDLDIFNYSSGMPSI |
| NO: 125 | FLLDAAPCEPESLEINKYFVVIIYALVFLLSLL | NO: 126 | LPDAVPCHSENLEINSYAVVVIYVLVTLLSL |
| (Human | GNSLVMLVILYSRVGRSVTDVYLLNLALADL | (Mouse | VGNSLVMLVILYNRSTCSVTDVYLLNLAIA |
| CXCR2) | LFALTLPIWAASKVNGWIFGTFLCKVVSLLK | CXCR2) | DLFFALTLPVWAASKVNGWTFGSTLCKIFS |
| | EVNFYSGILLLACISVDRYLAIVHATRTLTQK | | YVKEVTFYSSVLLLACISMDRYLAIVHATS |
| | RYLVKFICLSIWGLSLLLALPVLLFRRTVYSS | | TLIQKRHLVKFVCIAMWLLSVILALPILILR |
| | NVSPACYEDMGNNTANWRMLLRILPQSFGFI | | NPVKVNLSTLVCYEDVGNNTSRLRVVLRIL |
| | VPLLIMLFCYGFTLRTLFKAHMGQKHRAMR | | PQTFGFLVPLLIMLFCYGFTLRTLFKAHMG |
| | VIFAVVLIFLLCWLPYNLVLLADTLMRTQVIQ | | QKHRAMRVIFAVVLVFLLCWLPYNLVLFT |
| | ETCERRNHIDRALDATEILGILHSCLNPLIYAF | | DTLMRTKLIKETCERRDDIDKALNATEILGF |
| | IGQKFRHGLLKILAIHGLISKDSLPKDSRPSFV | | LHSCLNPIIYAFIGQKFRHGLLKIMATYGLV |
| | GSSSGHTSTTL | | SKEFLAKEGRPSFVSSSSANTSTTL |
| SEQ ID | MQSFNFEDFWENEDFSNYSYSSDLPPSLPDV | SEQ ID | MVLEVSDHQVLNDAEVAALLENFSSSYDY |
| NO: 127 | APCRPESLEINKYFVVIIYALVFLLSLLGNSLV | NO: 128 | GENESDSCCTSPPCPQDFSLNFDRAFLPALY |
| (cynomolgus | MLVILHSRVGRSITDVYLLNLAMADLLFALT | (human | SLLFLLGLLGNGAVAAVLLSRRTALSSTDT |
| CXCR2) | LPIWAAAKVNGWIFGTFLCKVVSLLKEVNFY | CXCR3) | FLLHLAVADTLLVLTLPLWAVDAAVQWVF |
| | SGILLLACISVDRYLAIVHATRTLTQKRYLVK | | GSGLCKVAGALFNINFYAGALLLACISFDR |
| | FVCLSIWSLSLLLALPVLLFRRTVYLTYISPVC | | YLNIVHATQLYRRGPPARVTLTCLAVWGL |
| | YEDMGNNTAKWRMVLRILPQTFGFILPLLIM | | CLLFALPDFIFLSAHHDERLNATHCQYNFP |
| | LFCYGFTLRTLFKAHMGQKHRAMRVIFAVV | | QVGRTALRVLQLVAGFLLPLLVMAYCYAH |
| | LIFLLCWLPYHLVLLADTLMRTRLINETCQRR | | ILAVLLVSRGQRRLRAMRLVVVVVVAFAL |
| | NNIDQALDATEILGILHSCLNPLIYAFIGQKFR | | CWTPYHLVVLVDILMDLGALARNCGRESR |
| | HGLLKILATHGLISKDSLPKDSRPSFVGSSSGH | | VDVAKSVTSGLGYMHCCLNPLLYAFVGVK |
| | TSTTL | | FRERMWMLLLRLGCPNQRGLQRQPSSSRR |
| | | | DSSWSETSEASYSGL |
| SEQ ID | MEGISIYTSDNYTEEMGSGDYDSMKEPCFRE | SEQ ID | MNYPLTLEMDLENLEDLFWELDRLDNYND |
| NO: 129 | ENANFNKIFLPTIYSIIFLTGIVGNGLVILVMG | NO: 130 | TSLVENHLCPATEGPLMASFKAVFVPVAYS |
| (human | YQKKLRSMTDKYRLHLSVADLLFVITLPFWA | (human | LIFLLGVIGNVLVLVILERHRQTRSSTETFLF |
| CXCR4) | VDAVANWYFGNFLCKAVHVIYTVNLYSSVLI | CXCR5) | HLAVADLLLVFILPFAVAEGSVGWVLGTFL |
| | LAFISLDRYLAIVHATNSQRPRKLLAEKVVY | | CKTVIALHKVNFYCSSLLLACIAVDRYLAIV |
| | VGVWIPALLLTIPDFIFANVSEADDRYICDRF | | HAVHAYRHRRLLSIHITCGTIWLVGFLLALP |
| | YPNDLWVVVFQFQHIMVGLILPGIVILSCYCII | | EILFAKVSQGHHNNSLPRCTFSQENQAETH |
| | ISKLSHSKGHQKRKALKTTVILILAFFACWLP | | AWFTSRFLYHVAGFLLPMLVMGWCYVGV |
| | YYIGISIDSFILLEIIKQGCEFENTVHKWISITEA | | VHRLRQAQRRPQRQKAVRVAILVTSIFFLC |
| | LAFFHCCLNPILYAFLGAKFKTSAQHALTSVS | | WSPYHIVIFLDTLARLKAVDNTCKLNGSLP |
| | RGSSLKILSKGKRGGHSSVSTESESSSFHSS | | VAITMCEFLGLAHCCLNPMLYTFAGVKFRS |
| | | | DLSRLLTKLGCTGPASLCQLFPSWRRSSLSE |
| | | | SENATSLTTF |
| SEQ ID | MAEHDYHEDYGFSSFNDSSQEEHQDFLQFSK | SEQ ID | MDLHLFDYSEPGNFSDISWPCNSSDCIVVD |
| NO: 131 | VFLPCMYLVVFVCGLVGNSLVLVISIFYHKL | NO: 132 | TVMCPNMPNKSVLLYTLSFIYIFIFVIGMIA |
| (human | QSLTDVFLVNLPLADLVFVCTLPFWAYAGIH | (human | NSVVVWVNIQAKTTGYDTHCYILNLAIADL |
| CXCR6) | EWVFGQVMCKSLLGIYTINFYTSMLILTCITV | CXCR7) | WVVLTIPVWVVSLVQHNQWPMGELTCKV |
| | DRFIVVVKATKAYNQQAKRMTWGKVTSLLI | | THLIFSINLFGSIFFLTCMSVDRYLSITYFTNT |
| | WVISLLVSLPQIIYGNVFNLDKLICGYHDEAIS | | PSSRKKMVRRVVCILVWLLAFCVSLPDTYY |
| | TVVLATQMTLGFFLPLLTMIVCYSVIIKTLLH | | LKTVTSASNNETYCRSFYPEHSIKEWLIGM |
| | AGGFQKHRSLKIIFLVMAVFLLTQMPFNLMK | | ELVSVVLGFAVPFSIIAVFYFLLARAISASSD |
| | FIRSTHWEYYAMTSFHYTIMVTEAIAYLRAC | | QEKHSSRKIIFSYVVVFLVCWLPYHVAVLL |
| | LNPVLYAFVSLKFRKNFWKLVKDIGCLPYLG | | DIFSILHYIPFTCRLEHALFTALHVTQCLSLV |
| | VSHQWKSSEDNSKTFSASHNVEATSMFQL | | HCCVNPVLYSFINRNYRYELMKAFIFKYSA |
| | | | KTGLTKLIDASRVSETEYSALEQSTK |
| SEQ ID | MSNITDPQMWDFDDLNFTGMPPADEDYSPC | | |
| NO: 133 | MLETETLNKYVVIIAYALVFLLSLLGNSLVM | | |
| (Human | LVILYSRVGRSVTDVYLLNLALADLLFALTLP | | |
| CXCR1) | IWAASKVNGWIFGTFLCKVVSLLKEVNFYSG | | |
| | ILLLACISVDRYLAIVHATRTLTQKRHLVKFV | | |
| | CLGCWGLSMNLSLPFFLFRQAYHPNNSSPVC | | |
| | YEVLGNDTAKWRMVLRILPHTFGFIVPLFVM | | |
| | LFCYGFTLRTLFKAHMGQKHRAMRVIFAVV | | |
| | LIFLLCWLPYNLVLLADTLMRTQVIQESCERR | | |
| | NNIGRALDATEILGFLHSCLNPIIYAFIGQNFR | | |
| | HGFLKILAMHGLVSKEFLARHRVTSYTSSSV | | |
| | NVSSNL | | |

Light chain constant regions

| | | | |
|---|---|---|---|
| SEQ ID | GQPKAAPSVTLFPPSSEELQANKATLVCLISD | SEQ ID | RTVAAPSVFIFPPSDEQLKSGTASVVCLLNN |
| NO: 134 | FYPGAVTVAWKADSSPVKAGVETTTPSKQS | NO: 135 | FYPREAKVQWKVDNALQSGNSQESVTEQD |
| (Human | NNKYAASSYLSLTPEQWKSHRSYSCQVTHEG | (Human | SKDSTYSLSSTLTLSKADYEKHKVYACEVT |
| Lambda | STVEKTVAPTECS | Kappa | HQGLSSPVTKSFNRGEC |
| light | | light | |
| chain | | chain | |
| constant | | constant | |
| region) | | region) | |

Additional BKO-4A8 single aa variants

| | | | |
|---|---|---|---|
| SEQ ID | EVQLLESGGGLVQPGGSLRLSCAASGFTFSSD | SEQ ID | EVQLLESGGGLVQPGGSLRLSCAASGFTFS |
| NO: 136 | TMSWVRQAPGKGLEWVSAISGRGRNTYYAD | NO: 137 | SSTMQWVRQAPGKGLEWVSAISGRGRNTY |
| (4A8 VH | SVKGRFTISRDNSRNTLYLQMNSLRAEDTAV | (4A8 VH | YADSVKGRFTISRDNSRNTLYLQMNSLRAE |
| S32D) | YYCAIQMGYWGQGILVTVSS | S35Q) | DTAVYYCAIQMGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 138 | TMDWVRQAPGKGLEWVSAISGRGRNTYYA | NO: 139 | SSTMKWVRQAPGKGLEWVSAISGRGRNTY |
| (4A8 VH | DSVKGRFTISRDNSRNTLYLQMNSLRAEDTA | (4A8 VH | YADSVKGRFTISRDNSRNTLYLQMNSLRAE |
| S35D) | VYYCAIQMGYWGQGILVTVSS | S35K) | DTAVYYCAIQMGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 140 | TMSWVRQAPGKGLEWVSSISGRGRNTYYAD | NO: 141 | SSTMSWVRQAPGKGLEWVSAISGRGQNTY |
| (4A8 VH | SVKGRFTISRDNSRNTLYLQMNSLRAEDTAV | (4A8 VH | YADSVKGRFTISRDNSRNTLYLQMNSLRAE |
| A50S) | YYCAIQMGYWGQGILVTVSS | R55Q) | DTAVYYCAIQMGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 142 | TMSWVRQAPGKGLEWVSAISGRGDNTYYAD | NO: 143 | SSTMSWVRQAPGKGLEWVSAISGRGHNTY |
| (4A8 VH | SVKGRFTISRDNSRNTLYLQMNSLRAEDTAV | (4A8 VH | YADSVKGRFTISRDNSRNTLYLQMNSLRAE |
| R55D) | YYCAIQMGYWGQGILVTVSS | R55H) | DTAVYYCAIQMGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 144 | TMSWVRQAPGKGLEWVSAISGRGRNTYYAD | NO: 145 | SSTMSWVRQAPGKGLEWVSAISGRGRNTY |
| (4A8 VH | SVKGRFTISRDNSRNTLYLQMNSLRAEDTAV | (4A8 VH | YADSVKGRFTISRDNSRNTLYLQMNSLRAE |
| M96S) | YYCAIQSGYWGQGILVTVSS | M96D) | DTAVYYCAIQDGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 146 | TMSWVRQAPGKGLEWVSAISGRGRNTYYAD | NO: 147 | SSTMSWVRQAPGKGLEWVSAISGRGRNTY |
| (4A8 VH | SVKGRFTISRDNSRNTLYLQMNSLRAEDTAV | (4A8 VH | YADSVKGRFTISRDNSRNTLYLQMNSLRAE |
| M96H) | YYCAIQHGYWGQGILVTVSS | M96L) | DTAVYYCAIQLGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 148 | TMSWVRQAPGKGLEWVSAISGRGRNTYYAD | NO: 149 | SSTMSWVRQAPGKGLEWVSAISGRGRNTY |
| (4A8 VH | SVKGRFTISRDNSRNTLYLQMNSLRAEDTAV | (4A8 VH | YADSVKGRFTISRDNSRNTLYLQMNSLRAE |
| M96W) | YYCAIQWGYWGQGILVTVSS | M96Y) | DTAVYYCAIQYGYWGQGILVTVSS |
| SEQ ID | EVQLLESGGGLVQPGGSLRLSCAASGFTFSSS | SEQ ID | EVQLLESGGGLVQPGGSLRLSCAASGFTFS |
| NO: 150 | TMSWVRQAPGKGLEWVSAISGRGRNTYYAD | NO: 151 | SSTMSWVRQAPGKGLEWVSAISGRGRNTY |
| (4A8 VH | SVKGRFTISRDNSRNTLYLQMNSLRAEDTAV | (4A8 VH | YADSVKGRFTISRDNSRNTLYLQMNSLRAE |
| Y102Q) | YYCAIQMGQWGQGILVTVSS | Y102D) | DTAVYYCAIQMGDWGQGILVTVSS |
| SEQ ID | QSALTQPPSASGSPGQSVTISCIGTSSDVGGY | SEQ ID | QSALTQPPSASGSPGQSVTISCIGTSSDVGG |
| NO: 152 | NYVSWYQQHPDKAPKLMIYEDNKRPSGVPD | NO: 153 | YNYVSWYQQHPDKAPKLMIYEYNKRPSGV |
| (4A8 VL | RFSGSKSGNTASLTVSGLQAEDEADYYCSSY | (4A8 VL | PDRFSGSKSGNTASLTVSGLQAEDEADYYC |
| V51D) | AGNNNFGVFGGGTKLTVL | V51Y) | SSYAGNNNFGVFGGGTKLTVL |
| SEQ ID | QSALTQPPSASGSPGQSVTISCIGTSSDVGGY | SEQ ID | QSALTQPPSASGSPGQSVTISCIGTSSDVGG |
| NO: 154 | NYVSWYQQHPDKAPKLMIYEVNKDPSGVPD | NO: 155 | YNYVSWYQQHPDKAPKLMIYEVNKRPSGV |
| (4A8 VL | RFSGSKSGNTASLTVSGLQAEDEADYYCSSY | (4A8 VL | PDRFSGSKSGNTASLTVSGLQAEDEADYYC |
| R54D) | AGNNNFGVFGGGTKLTVL | Y91S) | SSSAGNNNFGVFGGGTKLTVL |
| SEQ ID | QSALTQPPSASGSPGQSVTISCIGTSSDVGGY | SEQ ID | QSALTQPPSASGSPGQSVTISCIGTSSDVGG |
| NO: 156 | NYVSWYQQHPDKAPKLMIYEVNKRPSGVPD | NO: 157 | YNYVSWYQQHPDKAPKLMIYEVNKRPSGV |
| (4A8 VL | RFSGSKSGNTASLTVSGLQAEDEADYYCSSH | (4A8 VL | PDRFSGSKSGNTASLTVSGLQAEDEADYYC |
| Y91H) | AGNNNFGVFGGGTKLTVL | N94H) | SSYAGHNNFGVFGGGTKLTVL |
| SEQ ID | QSALTQPPSASGSPGQSVTISCIGTSSDVGGY | SEQ ID | QSALTQPPSASGSPGQSVTISCIGTSSDVGG |
| NO: 158 | NYVSWYQQHPDKAPKLMIYEVNKRPSGVPD | NO: 159 | YNYVSWYQQHPDKAPKLMIYEVNKRPSGV |
| (4A8 VL | RFSGSKSGNTASLTVSGLQAEDEADYYCSSY | (4A8 VL | PDRFSGSKSGNTASLTVSGLQAEDEADYYC |
| N95aS) | AGNNSFGVFGGGTKLTVL | N95aW) | SSYAGNNWFGVFGGGTKLTVL |
| SEQ ID | QSALTQPPSASGSPGQSVTISCIGTSSDVGGY | SEQ ID | QSALTQPPSASGSPGQSVTISCIGTSSDVGG |
| NO: 160 | NYVSWYQQHPDKAPKLMIYEVNKRPSGVPD | NO: 161 | YNYVSWYQQHPDKAPKLMIYEVNKRPSGV |
| (4A8 VL | RFSGSKSGNTASLTVSGLQAEDEADYYCSSY | (4A8 VL | PDRFSGSKSGNTASLTVSGLQAEDEADYYC |
| V97S) | AGNNNFGSFGGGTKLTVL | V97D) | SSYAGNNNFGDFGGGTKLTVL |

Additional BKO-4A8 combinatorial variants

| | | | |
|---|---|---|---|
| SEQ ID | EVQLLESGGGLVQPGGSLRLSCAASGFTFSSS | SEQ ID | EVQLLESGGGLVQPGGSLRLSCAASGFTFS |
| NO: 162 | TQSWVRQPPGKGLEWVSAISGRGRSTYYADS | NO: 163 | SSTQSWVRQPPGKGLEWVSAISGSGGSTYY |
| 4A8 VH | VKGRFTISRDNSKNTLYLQMNSLRAEDTAVY | 4A8 VH | ADSVKGRFTISRDNSKNTLYLQMNSLRAED |
| Variant | YCAIQEGYWGQGILVTVSS | Variant | TAVYYCAIQMGYWGQGILVTVSS |
| 102 | | 103 | |
| M34Q_ | | M34Q_ | |
| A40P_ | | A40P_ | |
| N56S_ | | R53S_ | |
| R75K_ | | R55G_ | |
| M96E | | N56S_ | |
| | | R75K | |
| SEQ ID | EVQLLESGGGLVQPGGSLRLSCAASGFTFSSS | SEQ ID | EVQLLESGGGLVQPGGSLRLSCAASGFTFS |
| NO: 164 | TQSWVRQPPGKGLEWVSAISGSGGSTYYADS | NO: 165 | SSTQSWVRQPPGKGLEWVSAISGSGGSTYY |
| 4A8 VH | VKGRFTISRDNSKNTLYLQMNSLRAEDTAVY | 4A8 VH | ADSVKGRFTISRDNSKNTLYLQMNSLRAED |
| Variant | YCAIQKGYWGQGILVTVSS | Variant | AVYYCAIQAGYWGQGILVTVSS |
| 104 | | 105 | |
| M34Q_ | | M34Q_ | |
| A40P_ | | A40P_ | |
| R53S_ | | R53S_ | |
| R55G_ | | R55G_ | |
| N56S_ | | N56S_ | |
| R75K_ | | R75K_ | |
| M96K | | M96A | |
| SEQ ID | EVQLLESGGGLVQPGGSLRLSCAASGFTFSSS | | |
| NO: 166 | TQSWVRQPPGKGLEWVSAISGSGGSTYYADS | | |
| 4A8 VH | VKGRFTISRDNSKNTLYLQMNSLRAEDTAVY | | |
| Variant | YCAIQEGYWGQGILVTVSS | | |
| 106 | | | |
| M34Q_ | | | |
| A40P_ | | | |
| R53S_ | | | |
| R55G_ | | | |
| N56S_ | | | |
| R75K_ | | | |
| M96E | | | |

Consensus Sequences

| | | | |
|---|---|---|---|
| SEQ ID | EVQLLESGGGLVQPGGSLRLSCAASGFTFSS | SEQ ID | QSALTQPPSASGSPGQSVTISCIGTSSDVGG |
| NO: 167 | X₁X₂X₃SWVRQAPGKGLEWVSAX₄SX₅X₆X₇ | NO: 168 | YNYVSWYQQHPDKAPKLMIYX₁₃VX₁₄X₁₅ |
| (4A8 | RX₈TYYADSVKGRFTISRDNSRNTLYLQMNS | (4A8 | X₁₆PSGVPDRFSGSKSGNTASLTVSGLQAED |
| Consensus | LRAEDTAVYYCAX₉QX₁₀X₁₁X₁₂WGQGILVTV | Consensus | EADYYCSSX₁₇AGX₁₈NX₁₉FGX₂₀FGGGTK |
| VH) | SS | VL) | LTVL |
| | Wherein: | | Wherein: |
| | X₁ is S, Q, H, L, W, or Y; X₂ is | | X₁₃ is E or D; X₁₄ is N, D, or |
| | T or A; and X₃ is M, Q, D, H, or | | S; X₁₅ is K, A, D, or H; and |
| | W | | X₁₆ is R or Q |
| | X₄ is I or H; X₅ is G or D; X₆ | | X₁₇ is Y or A; X₁₈ is N, A, S, |
| | is R, S, or Q; X₇ is G or D; and | | K, L, W, or Y; X₁₉ is N, Q, D, |
| | X₈ is N or S | | H, K, L, or Y; and X₂₀ is V, |
| | X₉ is I or K, X₁₀ is M, A, Q, or | | A, or K |
| | K; X₁₁ is G or D; X₁₂ is Y, S, | | |
| | or K | | |
| SEQ ID | SX₁X₂X₃S wherein: X₁ is S, Q, H, | SEQ ID | AX₄SX₅X₆X₇RX₈TYYADSVKG wherein: |
| NO: 169 | L, W, or Y; X₂ is T or A; and | NO: 170 | X₄ is I or H; X₅ is G or D; X₆ |
| (Consensus | X₃ is M, Q, D, H, or W | (Consensus | is R, S, or Q; X₇ is G or D; |
| VH CDR1) | | VH CDR2) | and X₈ is N or S |
| SEQ ID | QX₁₀X₁₁X₁₂ wherein X₁₀ is M, A, | SEQ ID | IGTSSDVGGYNYVS |
| NO: 171 | Q, or K; X₁₁ is G or D; and X₁₂ | NO: 172 | |
| (Consensus | is Y, S, or K | (Consensus | |
| VH CDR3) | | VL CDR1) | |
| SEQ ID | X₁₃VX₁₄X₁₅X₁₆PS wherein: X₁₃ is | SEQ ID | SSX₁₇AGX₁₈NX₁₉FGX₂₀ wherein: |
| NO: 173 | E or D; X₁₄ is N, D, or S; X₁₅ | NO: 174 | X₁₇ is Y or A; X₁₈ is N, A, S, |
| (Consensus | is K, A, D, or H; and X₁₆ is R | (Consensus | K, L, W, or Y; X₁₉ is N, Q, D, |
| VL CDR2) | or Q | VL CDR3) | H, K, L, or Y; and X₂₀ is V, A, |
| | | | or K |

CDR Sequences

| | | | |
|---|---|---|---|
| SEQ ID | SSTMS | SEQ ID | SQTMS |
| NO: 175 | | NO: 176 | |
| (4A8 | | (4A8 | |
| VHCDR1) | | 532Q | |
| | | VHCDR1) | |
| SEQ ID | SHTMS | SEQ ID | SLTMS |
| NO: 177 | | NO: 178 | |
| (4A8 | | (4A8 | |
| 532H | | 532L | |
| VHCDR1) | | VHCDR1) | |
| SEQ ID | SWTMS | SEQ ID | SYTMS |
| NO: 179 | | NO: 180 | |
| (4A8 | | (4A8 | |
| S32W | | S32Y | |
| VHCDR1) | | VHCDR1) | |
| SEQ ID | SSAMS | SEQ ID | SSTQS |
| NO: 181 | | NO: 182 | |
| (4A8 | | (4A8 | |
| T33A | | M34Q | |
| VHCDR1) | | VHCDR1) | |
| SEQ ID | SSTDS | SEQ ID | SSTHS |
| NO: 183 | | NO: 184 | |
| (4A8 | | (4A8 | |
| M34D | | M34H | |
| VHCDR1) | | VHCDR1) | |
| SEQ ID | SSTWS | SEQ ID | AISGRGRNTYYADSVKG |
| NO: 185 | | NO: 186 | |
| (4A8 | | (4A8 | |
| M34W | | VHCDR2) | |
| VHCDR1) | | | |
| SEQ ID | AHSGRGRNTYYADSVKG | SEQ ID | AISDRGRNTYYADSVKG |
| NO: 187 | | NO: 188 | |
| 4A8 | | 4A8 | |
| I51H | | G52aD | |
| VHCDR2 | | VHCDR2 | |
| SEQ ID | AISGSGRNTYYADSVKG | SEQ ID | AISGQGRNTYYADSVKG |
| NO: 189 | | NO: 190 | |
| 4A8 | | 4A8 | |
| R535 | | R53Q | |
| VHCDR2 | | VHCDR2 | |
| SEQ ID | AISGRDRNTYYADSVKG | SEQ ID | AISGRGRSTYYADSVKG |
| NO: 191 | | NO: 192 | |
| 4A8 | | 4A8 | |
| G54D | | N56S | |
| VHCDR2 | | VHCDR2 | |
| SEQ ID | AISGSGGSTYYADSVKG | SEQ ID | QMGY |
| NO: 193 | | NO: 194 | |
| 4A8 | | 4A8 | |
| 103, | | VHCDR3 | |
| 104, | | | |
| 105 | | | |
| VHCDR2 | | | |
| SEQ ID | QAGY | SEQ ID | QQGY |
| NO: 195 | | NO: 196 | |
| 4A8 | | 4A8 | |
| M96A | | M96Q | |
| VHCDR3 | | VHCDR3 | |
| SEQ ID | QKGY | SEQ ID | QMDY |
| NO: 197 | | NO: 198 | |
| 4A8 | | 4A8 | |
| M96K | | G101D | |
| VHCDR3 | | VHCDR3 | |
| SEQ ID | QMGS | SEQ ID | QMGK |
| NO: 199 | | NO: 200 | |
| 4A8 | | 4A8 | |
| Y102S | | Y102K | |
| VHCDR3 | | VHCDR3 | |
| SEQ ID | IGTSSDVGGYNYVS | SEQ ID | EVNKRPS |
| NO: 201 | | NO: 202 | |
| 4A8 | | 4A8 | |
| VLCDR1 | | VLCDR2 | |
| SEQ ID | DVNKRPS | SEQ ID | EVDKRPS |
| NO: 203 | | NO: 204 | |
| 4A8 | | 4A8 | |
| E50D | | N52D | |
| VLCDR2 | | VLCDR2 | |
| SEQ ID | EVSKRPS | SEQ ID | EVNARPS |
| NO: 205 | | NO: 206 | |
| 4A8 | | 4A8 | |
| N52A | | K53A | |
| VLCDR2 | | VLCDR2 | |
| SEQ ID | EVNDRPS | SEQ ID | EVNHRPS |
| NO: 207 | | NO: 208 | |
| 4A8 | | 4A8 | |
| K53D | | K53H | |
| VLCDR2 | | VLCDR2 | |
| SEQ ID | EVNKQPS | SEQ ID | SSYAGNNNFGV |
| NO: 209 | | NO: 210 | |
| 4A8 | | 4A8 | |
| R54Q | | VLCDR3 | |
| VLCDR2 | | | |
| SEQ ID | SSAAGNNNFGV | SEQ ID | SSYAGANNFGV |
| NO: 211 | | NO: 212 | |
| 4A8 | | 4A8 | |
| Y91A | | N94A | |
| VLCDR3 | | VLCDR3 | |
| SEQ ID | SSYAGSNNFGV | | |
| NO: 213 | | | |
| 4A8 | | | |
| N94S | | | |
| VLCDR3 | | | |
| SEQ ID | SSYAGKNNFGV | SEQ ID | SSYAGLNNFGV |
| NO: 214 | | NO: 215 | |
| 4A8 | | 4A8 | |
| N94K | | N94L | |
| VLCDR3 | | VLCDR3 | |
| SEQ ID | SSYAGWNNFGV | SEQ ID | SSYAGYNNFGV |
| NO: 216 | | NO: 217 | |
| 4A8 | | 4A8 | |
| N94W | | N94Y | |
| VLCDR3 | | VLCDR3 | |
| SEQ ID | SSYAGNNQFGV | SEQ ID | SSYAGNNDFGV |
| NO: 218 | | NO: 219 | |
| 4A8 | | 4A8 | |
| N95aQ | | N95aD | |
| VLCDR3 | | VLCDR3 | |
| SEQ ID | SSYAGNNHFGV | SEQ ID | SSYAGNNKFGV |
| NO: 220 | | NO: 221 | |
| 4A8 | | 4A8 | |
| N95aH | | N95aK | |
| VLCDR3 | | VLCDR3 | |
| SEQ ID | SSYAGNNLFGV | SEQ ID | SSYAGNNYFGV |
| NO: 222 | | NO: 223 | |
| 4A8 | | A48 | |
| N95aL | | N95aY | |
| VLCDR3 | | VLCDR3 | |
| SEQ ID | SSYAGNNNFGA | SEQ ID | SSYAGNNNFGK |
| NO: 224 | | NO: 225 | |
| 4A8 | | 4A8 | |
| V97A | | V97K | |
| VLCDR3 | | VLCDR3 | |

Consensus Sequences

| | | | |
|---|---|---|---|
| SEQ ID | EVQLLESGGGLVQPGGSLRLSCAASGFTFSSS | SEQ ID | QSALTQPPSASGSPGQSVTISCIGTSSDVGG |
| NO: 226 | TX₂₁SWVRQX₂₂PGKGLEWVSAISGX₂₃GX₂₄ | NO: 227 | YNYVSWYQQHPX₂₉KAPKLMIYEVX₃₀KRPS |
| (4A8 | X₂₅TYYADSVKGRFTISRDNSX₂₆NTLYLQMN | (4A8 | GVPDRFSGSKSGNTASLTVSGLQAEDEADY |
| Consensus | SLRAEDTAVYYCAX₂₇QX₂₈GYWGQGILVTVSS | Consensus | YCSSYAGX₃₁NNFGVFGGGTKLTVL |
| VH) | Wherein: | VL) | Wherein: |
| | X₂₁ iS M or Q; | | X₂₉ is D or G; |
| | X₂₂ is A or P; | | X₃₀ is N or S; |
| | X₂₃ is R or S; | | X₃₁ is N or S |
| | X₂₄ is R or G; and | | |
| | X₂₅ is N or S; | | |
| | X₂₆ is R or K; | | |
| | X₂₇ is I or K; | | |
| | X₂₈ is M, K, or A | | |
| SEQ ID | SSTX₂₁S wherein: X₂₁ is M or Q | SEQ ID | AISGX₂₃GX₂₄X₂₅TYYADSVKG |
| NO: 228 | | NO: 229 | wherein: X₂₃ is R or S; |
| (Consensus | | (Consensus | X₂₄ is R or G; and |
| VH CDR1) | | VH CDR2) | X₂₅ is N or S |
| SEQ ID | QX₂₈GY wherein: X₂₈ is M, K, or A | SEQ ID | IGTSSDVGGYNYVS |
| NO: 230 | | NO: 201 | |
| (Consensus | | (Consensus | |
| VH CDR3) | | VL CDR1) | |
| SEQ ID | EVX₃₀KRPS wherein: X₃₀ is N or S | SEQ ID | SSYAGX₃₁NNFGV wherein: |
| NO: 231 | | NO: 232 | X₃₁ is N or S |
| (Consensus | | (Consensus | |
| VL CDR2) | | VL CDR3) | |

Polynucleotide sequences

| | | | |
|---|---|---|---|
| BKO- | GAAGTGCAGCTGCTGGAATCTGGCGGAGGACTGG | 4A8 VH | GAAGTGCAGCTGCTGGAATCTGGCGGAGGACTG |
| 4A8 VH | TGCAGCCTGGCGGCAGCCTGAGACTGTCTTGTGC | Variant | GTGCAGCCTGGCGGCAGCCTGAGACTGTCTTGT |
| SEQ ID | CGCCAGCGGCTTCACCTTCAGCAGCAGCACAATG | 1 | GCCGCCAGCGGCTTCACCTTCAGCAGCAGCACA |
| NO: 233 | AGCTGGGTCCGACAGGCCCCTGGCAAGGGACTGG | SEQ ID | CAGAGCTGGGTCCGACAGGCCCCTGGCAAGGGA |
| | AATGGGTGTCCGCCATCAGCGGCAGAGGCCGGAA | NO: 234 | CTGGAATGGGTGTCCGCCATCAGCGGCAGAGGC |
| | CACCTACTACGCCGACAGCGTGAAGGGCCGGTTC | | CGGAGTACCTACTACGCCGACAGCGTGAAGGGC |
| | ACCATCAGCCGGGACAACAGCAGAAACACCCTGT | | CGGTTCACCATCAGCCGGGACAACAGCAGAAAC |
| | ACCTGCAGATGAACAGCCTGCGGGCCGAGGACAC | | ACCCTGTACCTGCAGATGAACAGCCTGCGGGCC |
| | CGCCGTGTACTACTGTGCCATCCAGATGGGCTAC | | GAGGACACCGCCGTGTACTACTGTGCCATCCAG |
| | TGGGGCCAGGGCATTCTCGTGACAGTGTCCTCA | | ATGGGCTACTGGGGCCAGGGCATTCTCGTGACA |
| | | | GTGTCCTCA |
| 4A8 VH | GAAGTGCAGCTGCTGGAATCTGGCGGAGGACTGG | 4A8 VH | GAAGTGCAGCTGCTGGAATCTGGCGGAGGACTG |
| Variant | TGCAGCCTGGCGGCAGCCTGAGACTGTCTTGTGC | Variant | GTGCAGCCTGGCGGCAGCCTGAGACTGTCTTGT |
| 2 | CGCCAGCGGCTTCACCTTCAGCAGCAGCACACAG | 3 | GCCGCCAGCGGCTTCACCTTCAGCAGCAGCACA |
| SEQ ID | AGCTGGGTCCGACAGCCTCCTGGCAAGGGACTGG | SEQ ID | CAGAGCTGGGTCCGACAGCCTCCTGGCAAGGGA |
| NO: 235 | AATGGGTGTCCGCCATCAGCGGCAGAGGCCGGAG | NO: 236 | CTGGAATGGGTGTCCGCCATCAGCGGCAGAGGC |
| | TACCTACTACGCCGACAGCGTGAAGGGCCGGTTC | | CGGAGTACCTACTACGCCGACAGCGTGAAGGGC |
| | ACCATCAGCCGGGACAACAGCAGAAACACCCTGT | | CGGTTCACCATCAGCCGGGACAACAGCAAAAAC |
| | ACCTGCAGATGAACAGCCTGCGGGCCGAGGACAC | | ACCCTGTACCTGCAGATGAACAGCCTGCGGGCC |
| | CGCCGTGTACTACTGTGCCATCCAGATGGGCTAC | | GAGGACACCGCCGTGTACTACTGTGCCATCCAG |
| | TGGGGCCAGGGCATTCTCGTGACAGTGTCCTCA | | ATGGGCTACTGGGGCCAGGGCATTCTCGTGACA |
| | | | GTGTCCTCA |
| 4A8 VH | GAAGTGCAGCTGCTGGAATCTGGCGGAGGACTGG | 4A8 VH | GAAGTGCAGCTGCTGGAATCTGGCGGAGGACTG |
| Variant | TGCAGCCTGGCGGCAGCCTGAGACTGTCTTGTGC | Variant | GTGCAGCCTGGCGGCAGCCTGAGACTGTCTTGT |
| 4 | CGCCAGCGGCTTCACCTTCAGCAGCAGCACACAG | 5 | GCCGCCAGCGGCTTCACCTTCAGCAGCAGCACA |
| SEQ ID | AGCTGGGTCCGACAGCCTCCTGGCAAGGGACTGG | SEQ ID | CAGAGCTGGGTCCGACAGCCTCCTGGCAAGGGA |
| NO: 237 | AATGGGTGTCCGCCATCAGCGGCAGAGGCCGGAG | NO: 238 | CTGGAATGGGTGTCCGCCATCAGCGGCAGAGGC |
| | TACCTACTACGCCGACAGCGTGAAGGGCCGGTTC | | CGGAGTACCTACTACGCCGACAGCGTGAAGGGC |
| | ACCATCAGCCGGGACAACAGCAGAAACACCCTGT | | CGGTTCACCATCAGCCGGGACAACAGCAAAAAC |
| | ACCTGCAGATGAACAGCCTGCGGGCCGAGGACAC | | ACCCTGTACCTGCAGATGAACAGCCTGCGGGCC |
| | CGCCGTGTACTACTGTGCCATCCAGAAGGGCTAC | | GAGGACACCGCCGTGTACTACTGTGCCATCCAG |
| | TGGGGCCAGGGCATTCTCGTGACAGTGTCCTCA | | AAGGGCTACTGGGGCCAGGGCATTCTCGTGACA |
| | | | GTGTCCTCA |
| 4A8 VH | GAAGTGCAGCTGCTGGAATCTGGCGGAGGACTGG | 4A8 VH | GAAGTGCAGCTGCTGGAATCTGGCGGAGGACTG |
| Variant | TGCAGCCTGGCGGCAGCCTGAGACTG | Variant | GTGCAGCCTGGCGGCAGCCTGAGACTG |
| 6 | TCTTGTGCCGCCAGCGGCTTCACCTTCAGCAGCA | 7 | TCTTGTGCCGCCAGCGGCTTCACCTTCAGCAGC |
| SEQ ID | GCACACAGAGCTGGGTCCGACAGCCTCCTGGCAA | SEQ ID | AGCACACAGAGCTGGGTCCGACAGCCTCCTGGC |
| NO: 239 | GGGACTGGAATGGGTGTCCGCCATCAGCGGCAGA | NO: 240 | AAGGGACTGGAATGGGTGTCCGCCATCAGCGGC |
| | GGCCGGAGTACCTACTACGCCGACAGCGTGAAGG | | AGAGGCCGGAGTACCTACTACGCCGACAGCGTG |
| | GCCGGTTCACCATCAGCCGGGACAACAGCAAAAA | | AAGGGCCGGTTCACCATCAGCCGGGACAACAGC |
| | CACCCTGTACCTGCAGATGAACAGCCTGCGGGCC | | AGAAACACCCTGTACCTGCAGATGAACAGCCTG |
| | GAGGACACCGCCGTGTACTACTGTGCCAAGCAGA | | CGGGCCGAGGACACCGCCGTGTACTACTGTGCC |
| | AGGGCTACTGGGGCCAGGGCATTCTCGTGACAGT | | AAGCAGAAGGGCTACTGGGGCCAGGGCATTCTC |
| | GTCCTCA | | GTGACAGTGTCCTCA |
| 4A8 VH | GAAGTGCAGCTGCTGGAATCTGGCGGAGGACTGG | 4A8 VH | GAAGTGCAGCTGCTGGAATCTGGCGGAGGACTG |
| Variant | TGCAGCCTGGCGGCAGCCTGAGACTGTCTTGTGC | Variant | GTGCAGCCTGGCGGCAGCCTGAGACTGTCTTGT |
| 8 | CGCCAGCGGCTTCACCTTCAGCAGCAGCACACAG | 9 | GCCGCCAGCGGCTTCACCTTCAGCAGCAGCACA |
| SEQ ID | AGCTGGGTCCGACAGGCCCCTGGCAAGGGACTGG | SEQ ID | CAGAGCTGGGTCCGACAGGCCCCTGGCAAGGGA |
| NO: 241 | AATGGGTGTCCGCCATCAGCGGCAGAGGCCGGAG | NO: 242 | CTGGAATGGGTGTCCGCCATCAGCGGCAGAGGC |
| | TACCTACTACGCCGACAGCGTGAAGGGCCGGTTC | | CGGAGTACCTACTACGCCGACAGCGTGAAGGGC |
| | ACCATCAGCCGGGACAACAGCAGAAACACCCTGT | | CGGTTCACCATCAGCCGGGACAACAGCAAAAAC |
| | ACCTGCAGATGAACAGCCTGCGGGCCGAGGACAC | | ACCCTGTACCTGCAGATGAACAGCCTGCGGGCC |
| | CGCCGTGTACTACTGTGCCATCCAGAAGGGCTAC | | GAGGACACCGCCGTGTACTACTGTGCCATCCAG |
| | TGGGGCCAGGGCATTCTCGTGACAGTGTCCTCA | | AAGGGCTACTGGGGCCAGGGCATTCTCGTGACA |
| | | | GTGTCCTCA |
| 4A8 VH | GAAGTGCAGCTGCTGGAATCTGGCGGAGGACTGG | 4A8 VH | GAAGTTCAGCTGCTTGAATCTGGCGGAGGACTG |
| Variant | TGCAGCCTGGCGGCAGCCTGAGACTGTCTTGTGC | Variant | GTTCAGCCTGGCGGATCTCTGAGACTGTCTTGT |
| 10 | CGCCAGCGGCTTCACCTTCAGCAGCAGCACAATG | 101 | GCCGCCAGCGGCTTCACCTTTAGCAGCAGCACA |
| SEQ ID | AGCTGGGTCCGACAGGCCCCTGGCAAGGGACTGG | SEQ ID | CAGAGCTGGGTCCGACAGCCTCCTGGCAAAGGA |
| NO: 243 | AATGGGTGTCCGCCATCAGCGGCAGAGGCCGGAA | NO: 244 | CTGGAATGGGTGTCCGCCATCTCTGGCAGAGGC |
| | CACCTACTACGCCGACAGCGTGAAGGGCCGGTTC | | AGAAGCACCTACTACGCCGACTCTGTGAAGGGC |
| | ACCATCAGCCGGGACAACAGCAGAAACACCCTGT | | AGATTCACCATCAGCCGGGACAACAGCAAGAAC |
| | ACCTGCAGATGAACAGCCTGCGGGCCGAGGACAC | | ACCCTGTACCTGCAGATGAACAGCCTGAGAGCC |
| | CGCCGTGTACTACTGTGCCAAGCAGAAGGGCTAC | | GAGGACACCGCCGTGTACTATTGTGCCATCCAG |
| | TGGGGCCAGGGCATTCTCGTGACAGTGTCCTCA | | GCCGGCTATTGGGGCCAGGGAATACTCGTGACA |
| | | | GTGTCCTCA |
| 4A8 VH | GAAGTTCAGCTGCTTGAATCTGGCGGAGGACTGG | 4A8 VH | GAAGTTCAGCTGCTTGAATCTGGCGGAGGACTG |
| Variant | TTCAGCCTGGCGGATCTCTGAGACTGTCTTGTGC | Variant | GTTCAGCCTGGCGGATCTCTGAGACTGTCTTGT |
| 103 | CGCCAGCGGCTTCACCTTTAGCAGCAGCACACAG | 104 | GCCGCCAGCGGCTTCACCTTTAGCAGCAGCACA |
| SEQ ID | AGCTGGGTCCGACAGCCTCCTGGCAAAGGACTGG | SEQ ID | CAGAGCTGGGTCCGACAGCCTCCTGGCAAAGGA |
| NO: 245 | AATGGGTGTCCGCCATCTCTGGCAGCGGCGGCAG | NO: 246 | CTGGAATGGGTGTCCGCCATCTCTGGCAGCGGC |
| | CACATATTACGCCGATTCTGTGAAGGGCAGATTC | | GGCAGCACATATTACGCCGATTCTGTGAAGGGC |
| | ACCATCAGCCGGGACAACAGCAAGAACACCCTGT | | AGATTCACCATCAGCCGGGACAACAGCAAGAAC |
| | ACCTGCAGATGAACAGCCTGAGAGCCGAGGACAC | | ACCCTGTACCTGCAGATGAACAGCCTGAGAGCC |
| | CGCCGTGTACTATTGCGCCATCCAGATGGGCTAT | | GAGGACACCGCCGTGTACTATTGCGCCATCCAG |
| | TGGGGCCAGGGAATCCTCGTGACAGTGTCCTCA | | AAAGGCTATTGGGGCCAGGGCATCCTCGTGACA |
| | | | GTGTCCTCA |
| 4A8 VH | GAAGTTCAGCTGCTTGAATCTGGCGGAGGACTGG | Human | GCTAGCACCAAGGGACCCAGCGTGTTCCCCCTG |
| Variant | TTCAGCCTGGCGGATCTCTGAGACTGTCTTGTGC | IgG1 | GCCCCCAGCAGCAAGAGCACATCTGGCGGAACA |
| 105 | CGCCAGCGGCTTCACCTTTAGCAGCAGCACACAG | SEQ ID | GCCGCCCTGGGCTGCCTGGTGAAAGACTACTTC |
| SEQ ID | AGCTGGGTCCGACAGCCTCCTGGCAAAGGACTGG | NO: 248 | CCCGAGCCCGTGACCGTGAGCTGGAACAGCGGA |
| NO: 247 | AATGGGTGTCCGCCATCTCTGGCAGCGGCGGCAG | | GCCCTGACCAGCGGCGTGCACACCTTTCCAGCC |
| | CACATATTACGCCGATTCTGTGAAGGGCAGATTC | | GTGCTGCAGAGCAGCGGCCTGTACAGCCTGAGC |
| | ACCATCAGCCGGGACAACAGCAAGAACACCCTGT | | AGCGTGGTGACAGTGCCCTCTAGCAGCCTGGGC |
| | ACCTGCAGATGAACAGCCTGAGAGCCGAGGACAC | | ACCCAGACCTACATCTGCAACGTGAACCACAAG |
| | CGCCGTGTACTATTGTGCCATCCAGGCCGGCTAT | | CCCAGCAACACCAAGGTGGACAAAAAGGTGGAA |
| | TGGGGCCAGGGAATACTCGTGACAGTGTCCTCA | | CCCAAGAGCTGCGACAAGACCCACACCTGTCCC |
| | | | CCCTGCCCTGCCCCTGAACTGCTGGGCGGACCC |
| | | | TCCGTGTTCCTGTTCCCCCCAAAGCCCAAGGAC |
| | | | ACCCTGATGATCAGCCGGACCCCCGAAGTGACC |
| | | | TGCGTGGTGGTGGACGTGTCCCACGAGGACCCT |
| | | | GAAGTGAAGTTCAATTGGTACGTGGACGGCGTG |
| | | | GAAGTGCACAACGCCAAGACCAAGCCCAGAGAG |
| | | | GAACAGTACAACAGCACCTACCGGGTGGTGTCC |
| | | | GTGCTGACCGTGCTGCACCAGGACTGGCTGAAC |
| | | | GGCAAAGAGTACAAGTGCAAGGTGTCCAACAAG |
| | | | GCCCTGCCTGCTCCCATCGAGAAAACCATCAGC |
| | | | AAGGCCAAGGGCCAGCCCCGCGAGCCTCAGGTG |
| | | | TACACACTGCCCCCCAGCCGGGACGAGCTGACC |
| | | | AAGAACCAGGTGTCCCTGACCTGTCTGGTGAAA |
| | | | GGCTTCTACCCCAGCGATATCGCCGTGGAATGG |
| | | | GAGAGCAACGGCCAGCCCGAGAACAACTACAAG |
| | | | ACCACCCCCCCTGTGCTGGACAGCGACGGCTCA |
| | | | TTCTTCCTGTACAGCAAGCTGACCGTGGACAAG |
| | | | AGCCGGTGGCAGCAGGGCAACGTGTTCAGCTGC |
| | | | AGCGTGATGCACGAGGCCCTGCACAACCACTAC |
| | | | ACCCAGAAGTCCCTGAGCCTGAGCCCCGGC |
| Human | GCTAGCACCAAGGGACCCAGCGTGTTCCCCCTGG | Human | GCTAGCACCAAGGGCCCCAGCGTGTTCCCCCTG |
| IgG1* | CCCCCAGCAGCAAGAGCACATCTGGCGGAACAGC | IgG4 | GCCCCTTGTAGCAGAAGCACCAGCGAGAGCACA |
| SEQ ID | CGCCCTGGGCTGCCTGGTGAAAGACTACTTCCCC | SEQ ID | GCCGCCCTGGGCTGCCTGGTGAAAGACTACTTC |
| NO: 249 | GAGCCCGTGACCGTGAGCTGGAACAGCGGAGCCC | NO: 250 | CCCGAGCCCGTCACCGTGTCCTGGAACAGCGGA |
| | TGACCAGCGGCGTGCACACCTTTCCAGCCGTGCT | | GCCCTGACCAGCGGCGTGCACACCTTTCCAGCC |
| | GCAGAGCAGCGGCCTGTACAGCCTGAGCAGCGTG | | GTGCTGCAGAGCAGCGGCCTGTACAGCCTGAGC |
| | GTGACAGTGCCCTCTAGCAGCCTGGGCACCCAGA | | AGCGTGGTGACAGTGCCCTCCAGCAGCCTGGGC |
| | CCTACATCTGCAACGTGAACCACAAGCCCAGCAA | | ACCAAGACCTACACCTGTAACGTGGACCACAAG |
| | CACCAAGGTGGACAAAAAGGTGGAACCCAAGAGC | | CCCAGCAACACCAAGGTGGACAAGCGGGTGGAA |
| | TGCGACAAGACCCACACCTGTCCCCCCTGCCCTG | | TCTAAGTACGGCCCACCCTGCCCCCCCTGCCCT |
| | CCCCTGAACTGGCTGGCGCTCCCTCCGTGTTCCT | | GCCCCTGAATTTCTGGGCGGACCCTCCGTGTTC |
| | GTTCCCCCCAAAGCCCAAGGACACCCTGATGATC | | CTGTTCCCCCCAAAGCCCAAGGACACCCTGATG |
| | AGCCGGACCCCCGAAGTGACCTGCGTGGTGGTGG | | ATCAGCCGGACCCCCGAAGTGACCTGCGTGGTG |
| | ACGTGTCCCACGAGGACCCTGAAGTGAAGTTCAA | | GTGGACGTGTCCCAGGAAGATCCCGAGGTCCAG |
| | TTGGTACGTGGACGGCGTGGAAGTGCACAACGCC | | TTCAATTGGTACGTGGACGGCGTGGAAGTGCAC |
| | AAGACCAAGCCCAGAGAGGAACAGTACAACAGCA | | AACGCCAAGACCAAGCCCAGAGAGGAACAGTTC |
| | CCTACCGGGTGGTGTCCGTGCTGACCGTGCTGCA | | AACAGCACCTACCGGGTGGTGTCCGTGCTGACC |
| | CCAGGACTGGCTGAACGGCAAAGAGTACAAGTGC | | GTGCTGCACCAGGACTGGCTGAACGGCAAAGAG |
| | AAGGTGTCCAACAAGGCCCTGCCTGCTCCCATCG | | TACAAGTGCAAAGTCTCCAACAAGGGCCTGCCC |
| | AGAAAACCATCAGCAAGGCCAAGGGCCAGCCCCG | | AGCTCCATCGAGAAAACCATCAGCAAGGCCAAG |
| | CGAGCCTCAGGTGTACACACTGCCCCCCAGCCGG | | GGCCAGCCCCGCGAGCCTCAGGTGTACACACTG |
| | GACGAGCTGACCAAGAACCAGGTGTCCCTGACCT | | CCCCCCAGCCAGGAAGAGATGACCAAGAACCAG |
| | GTCTGGTGAAAGGCTTCTACCCCAGCGATATCGC | | GTGTCCCTGACCTGTCTGGTGAAAGGCTTCTAC |
| | CGTGGAATGGGAGAGCAACGGCCAGCCCGAGAAC | | CCCAGCGATATCGCCGTGGAATGGGAGAGCAAC |
| | AACTACAAGACCACCCCCCCTGTGCTGGACAGCG | | GGCCAGCCCGAGAACAACTACAAGACCACCCCC |
| | ACGGCTCATTCTTCCTGTACAGCAAGCTGACCGT | | CCTGTGCTGGACAGCGACGGCAGCTTCTTCCTG |
| | GGACAAGAGCCGGTGGCAGCAGGGCAACGTGTTC | | TACTCCCGGCTGACCGTGGACAAGAGCCGGTGG |
| | AGCTGCAGCGTGATGCACGAGGCCCTGCACAACC | | CAGGAAGGCAACGTCTTCAGCTGCAGCGTGATG |
| | ACTACACCCAGAAGTCCCTGAGCCTGAGCCCCGG | | CACGAGGCCCTGCACAACCACTACACCCAGAAG |
| | C | | TCCCTGAGCCTGAGCCTGGGC |
| Human | GCTAGCACCAAGGGCCCCAGCGTGTTCCCCCTGG | Human | GCTAGCACCAAGGGCCCCAGCGTGTTCCCTCTG |
| IgG4* | CCCCTTGTAGCAGAAGCACCAGCGAGAGCACAGC | IgG2* | GCCCCTTGTAGCAGAAGCACCAGCGAGTCTACA |
| SEQ ID | CGCCCTGGGCTGCCTGGTGAAAGACTACTTCCCC | SEQ ID | GCCGCCCTGGGCTGCCTCGTGAAGGACTACTTT |
| NO: 251 | GAGCCCGTCACCGTGTCCTGGAACAGCGGAGCCC | NO: 252 | CCCGAGCCCGTCACCGTGTCCTGGAACTCTGGC |
| | TGACCAGCGGCGTGCACACCTTTCCAGCCGTGCT | | GCTCTGACAAGCGGCGTGCACACCTTTCCAGCC |
| | GCAGAGCAGCGGCCTGTACAGCCTGAGCAGCGTG | | GTGCTGCAGAGCAGCGGCCTGTACTCTCTGAGC |
| | GTGACAGTGCCCTCCAGCAGCCTGGGCACCAAGA | | AGCGTCGTGACCGTGCCCAGCAGCAATTTCGGC |
| | CCTACACCTGTAACGTGGACCACAAGCCCAGCAA | | ACCCAGACCTACACCTGTAACGTGGACCACAAG |
| | CACCAAGGTGGACAAGCGGGTGGAATCTAAGTAC | | CCCAGCAACACCAAGGTGGACAAGACCGTGGAA |
| | GGCCCACCCTGCCCCCCCTGCCCTGCCCCTGAAT | | CGGAAGTGCTGCGTGGAATGCCCCCCTTGTCCT |
| | TTCTGGGCGGACCCTCCGTGTTCCTGTTCCCCCC | | GCCCCTCCAGTGGCTGGCCCTTCCGTGTTCCTG |
| | AAAGCCCAAGGACACCCTGTATATCACTCGGGAG | | TTCCCCCCAAAGCCCAAGGACACCCTGATGATC |
| | CCCGAAGTGACCTGCGTGGTGGTGGACGTGTCCC | | AGCCGGACCCCCGAAGTGACCTGCGTGGTGGTG |
| | AGGAAGATCCCGAGGTCCAGTTCAATTGGTACGT | | GATGTGTCCCACGAGGACCCCGAGGTGCAGTTC |
| | GGACGGCGTGGAAGTGCACAACGCCAAGACCAAG | | AATTGGTACGTGGACGGCGTGGAAGTGCACAAC |
| | CCCAGAGAGGAACAGTTCAACAGCACCTACCGGG | | GCCAAGACCAAGCCCAGAGAGGAACAGTTCAAC |
| | TGGTGTCCGTGCTGACCGTGCTGCACCAGGACTG | | AGCACCTTCCGGGTGGTGTCCGTGCTGACCGTG |
| | GCTGAACGGCAAAGAGTACAAGTGCAAAGTCTCC | | GTGCATCAGGACTGGCTGAACGGCAAAGAGTAC |
| | AACAAGGGCCTGCCCAGCTCCATCGAGAAAACCA | | AAGTGCAAGGTGTCCAACAAGGGCCTGCCCAGC |
| | TCAGCAAGGCCAAGGGCCAGCCCCGCGAGCCTCA | | TCCATCGAGAAAACCATCAGCAAGACCAAAGGC |
| | GGTGTACACACTGCCCCCCAGCCAGGAAGAGATG | | CAGCCCCGCGAGCCCCAGGTGTACACACTGCCT |
| | ACCAAGAACCAGGTGTCCCTGACCTGTCTGGTGA | | CCAAGCCGGGAAGAGATGACCAAGAATCAGGTG |
| | AAGGCTTCTACCCCAGCGATATCGCCGTGGAATG | | TCCCTGACCTGTCTCGTGAAAGGCTTCTACCCC |
| | GGAGAGCAACGGCCAGCCCGAGAACAACTACAAG | | TCCGATATCGCCGTGGAATGGGAGAGCAACGGC |
| | ACCACCCCCCCTGTGCTGGACAGCGACGGCAGCT | | CAGCCCGAGAACAACTACAAGACCACCCCCCCC |
| | TCTTCCTGTACTCCCGGCTGACCGTGGACAAGAG | | ATGCTGGACAGCGACGGCTCATTCTTCCTGTAC |
| | CCGGTGGCAGGAAGGCAACGTCTTCAGCTGCAGC | | AGCAAGCTGACAGTGGACAAGTCCCGGTGGCAG |
| | GTGATGCACGAGGCCCTGCACAACCACTACACCC | | CAGGGCAACGTGTTCAGCTGCAGCGTGATGCAC |
| | AGAAGTCCCTGAGCCTGAGCCTGGGC | | GAGGCCCTGCACAACCACTACACCCAGAAGTCC |
| | | | CTGAGCCTGAGCCCTGGC |
| BKO- | CAGTCTGCTCTGACACAGCCTCCTAGCGCCTCTG | 4A8 VL | CAGTCTGCTCTGACACAGCCTCCTAGCGCCTCT |
| 4A8 VL | GCTCTCCTGGCCAGAGCGTGACCATCAGCTGTAT | variant | GGCTCTCCTGGCCAGAGCGTGACCATCAGCTGT |
| SEQ ID | CGGCACCAGCAGCGACGTGGGCGGCTACAACTAC | b | ATCGGCACCAGCAGCGACGTGGGCGGCTACAAC |
| NO: 253 | GTGTCCTGGTATCAGCAGCACCCCGACAAGGCCC | SEQ ID | TACGTGTCCTGGTATCAGCAGCACCCCGACAAG |
| | CCAAGCTGATGATCTACGAAGTGAACAAGCGGCC | NO: 254 | GCCCCCAAGCTGATGATCTACGAAGTGTCCAAG |
| | CAGCGGCGTGCCCGATAGATTCAGCGGCAGCAAG | | CGGCCCAGCGGCGTGCCCGATAGATTCAGCGGC |
| | AGCGGCAACACCGCCAGCCTCACAGTGTCTGGAC | | AGCAAGAGCGGCAACACCGCCAGCCTCACAGTG |
| | TGCAGGCCGAGGACGAGGCCGACTACTACTGTAG | | TCTGGACTGCAGGCCGAGGACGAGGCCGACTAC |
| | CAGCTACGCCGGCAACAACAACTTCGGCGTGTTC | | TACTGTAGCAGCTACGCCGGCAGCAACAACTTC |
| | GGCGGAGGCACCAAGCTGACAGTCCTA | | GGCGTGTTCGGCGGAGGCACCAAGCTGACAGTC |
| | | | CTA |
| 4A8 VL | CAGTCTGCTCTGACACAGCCTCCTAGCGCCTCTG | lambda | GGTCAGCCCAAGGCCGCTCCCAGCGTGACCCTG |
| variant | GCTCTCCTGGCCAGAGCGTGACCATCAGCTGTAT | constant | TTCCCCCCAAGCAGCGAGGAACTGCAGGCCAAC |
| c | CGGCACCAGCAGCGACGTGGGCGGCTACAACTAC | light | AAGGCCACCCTGGTGTGCCTGATCAGCGACTTC |
| SEQ ID | GTGTCCTGGTATCAGCAGCACCCCGgTAAGGCCC | chain | TACCCTGGGGCCGTGACCGTGGCCTGGAAGGCC |
| NO: 255 | CCAAGCTGATGATCTACGAAGTGTCCAAGCGGCC | SEQ ID | GATAGCAGCCCTGTGAAGGCCGGCGTGGAAACC |
| | CAGCGGCGTGCCCGATAGATTCAGCGGCAGCAAG | NO: 256 | ACCACCCCCTCCAAGCAGAGCAACAACAAATAC |
| | AGCGGCAACACCGCCAGCCTCACAGTGTCTGGAC | | GCCGCCAGCAGCTACCTGTCCCTGACCCCCGAG |
| | TGCAGGCCGAGGACGAGGCCGACTACTACTGTAG | | CAGTGGAAGTCCCACCGGTCCTACAGCTGCCAG |
| | CAGCTACGCCGGCAgCAACAACTTCGGCGTGTTC | | GTGACACACGAGGGCAGCACCGTGGAAAAGACC |
| | GGCGGAGGCACCAAGCTGACAGTCCTA | | GTGGCCCCCACCGAGTGCAGC |

What is claimed:

1. A human antibody molecule that immunospecifically binds to human CXCR2, wherein the antibody molecule comprises:
the heavy chain CDR1, CDR2, and CDR3 of SEQ ID NO: 167 and the light chain CDR1, CDR2, and CDR3 of SEQ ID NO: 168; or
the heavy chain CDR1, CDR2, and CDR3 of SEQ ID NO: 226 and the light chain CDR1, CDR2, and CDR3 of SEQ ID NO: 227
and wherein the antibody molecule inhibits CXCL1-induced activation of CXCR2 or CXCL5-induced activation of CXCR2.

2. The human antibody molecule of claim 1, wherein:
the heavy chain CDR1 comprises the amino acid sequence of SEQ ID NO: 169, the heavy chain CDR2 comprises the amino acid sequence of SEQ ID NO: 170, the heavy chain CDR3 comprises the amino acid sequence of SEQ ID NO: 171, the light chain CDR1 comprises the amino acid sequence of SEQ ID NO: 172, the light chain CDR2 comprises the amino acid sequence of SEQ ID NO: 173, and the light chain CDR3 comprises the amino acid sequence of SEQ ID NO: 174; or
the heavy chain CDR1 comprises the amino acid sequence of SEQ ID NO: 228, the heavy chain CDR2 comprises the amino acid sequence of SEQ ID NO: 229, the heavy chain CDR3 comprises the amino acid sequence of SEQ ID NO: 230, the light chain CDR1 comprises the amino acid sequence of SEQ ID NO: 201, the light chain CDR2 comprises the amino acid sequence of SEQ ID NO: 231, and the light chain CDR3 comprises the amino acid sequence of SEQ ID NO: 232.

3. The human antibody molecule of claim 2, wherein:
the heavy chain variable region comprises the amino acid sequence of SEQ ID NO: 167 and the light chain variable region comprises the amino acid sequence of SEQ ID NO: 168; or
the heavy chain variable region comprises the amino acid sequence of SEQ ID NO: 226 and the light chain variable region comprises the amino acid sequence of SEQ ID NO: 227.

4. The human antibody molecule of claim 3, wherein:
a) the heavy chain variable region comprises the amino acid sequence of SEQ ID NO: 98 and the light chain variable region comprises the amino acid sequence of SEQ ID NO: 109 or 110;
b) the heavy chain variable region comprises the amino acid sequence of SEQ ID NO: 99 and the light chain variable region comprises the amino acid sequence of SEQ ID NO: 109 or 110;
c) the heavy chain variable region comprises the amino acid sequence of SEQ ID NO: 100 and the light chain variable region comprises the amino acid sequence of SEQ ID NO: 109 or 110;
d) the heavy chain variable region comprises the amino acid sequence of SEQ ID NO: 101 and the light chain variable region comprises the amino acid sequence of SEQ ID NO: 109 or 110;
e) the heavy chain variable region comprises the amino acid sequence of SEQ ID NO: 102 and the light chain variable region comprises the amino acid sequence of SEQ ID NO: 109 or 110;
f) the heavy chain variable region comprises the amino acid sequence of SEQ ID NO: 103 and the light chain variable region comprises the amino acid sequence of SEQ ID NO: 109 or 110;
g) the heavy chain variable region comprises the amino acid sequence of SEQ ID NO: 104 and the light chain variable region comprises the amino acid sequence of SEQ ID NO: 109 or 110;
h) the heavy chain variable region comprises the amino acid sequence of SEQ ID NO: 105 and the light chain variable region comprises the amino acid sequence of SEQ ID NO: 109 or 110;
i) the heavy chain variable region comprises the amino acid sequence of SEQ ID NO: 106 and the light chain variable region comprises the amino acid sequence of SEQ ID NO: 109 or 110;
j) the heavy chain variable region comprises the amino acid sequence of SEQ ID NO: 107 and the light chain variable region comprises the amino acid sequence of SEQ ID NO: 109 or 110;
k) the heavy chain variable region comprises the amino acid sequence of SEQ ID NO: 108 and the light chain variable region comprises the amino acid sequence of SEQ ID NO: 109 or 110;
l) the heavy chain variable region comprises the amino acid sequence of SEQ ID NO: 162 and the light chain variable region comprises the amino acid sequence of SEQ ID NO: 109 or 110;
m) the heavy chain variable region comprises the amino acid sequence of SEQ ID NO: 163 and the light chain variable region comprises the amino acid sequence of SEQ ID NO: 109 or 110;
n) the heavy chain variable region comprises the amino acid sequence of SEQ ID NO: 164 and the light chain variable region comprises the amino acid sequence of SEQ ID NO: 109 or 110;
o) the heavy chain variable region comprises the amino acid sequence of SEQ ID NO: 165 and the light chain variable region comprises the amino acid sequence of SEQ ID NO: 109 or 110; or
p) the heavy chain variable region comprises the amino acid sequence of SEQ ID NO: 166 and the light chain variable region comprises the amino acid sequence of SEQ ID NO: 109 or 110.

5. The human antibody molecule of claim 4, wherein:
a) the heavy chain variable region comprises the amino acid sequence of SEQ ID NO: 108 and the light chain variable region comprises the amino acid sequence of SEQ ID NO: 110;
b) the heavy chain variable region comprises the amino acid sequence of SEQ ID NO: 162 and the light chain variable region comprises the amino acid sequence of SEQ ID NO: 110;
c) the heavy chain variable region comprises the amino acid sequence of SEQ ID NO: 163 and the light chain variable region comprises the amino acid sequence of SEQ ID NO: 110;
d) the heavy chain variable region comprises the amino acid sequence of SEQ ID NO: 164 and the light chain variable region comprises the amino acid sequence of SEQ ID NO: 110;
e) the heavy chain variable region comprises the amino acid sequence of SEQ ID NO: 165 and the light chain variable region comprises the amino acid sequence of SEQ ID NO: 110; or
f) the heavy chain variable region comprises the amino acid sequence of SEQ ID NO: 166 and the light chain variable region comprises the amino acid sequence of SEQ ID NO: 110.

6. The human antibody molecule of claim 1, wherein the heavy chain CDR1 comprises the amino acid sequence of SEQ ID NO: 182, the heavy chain CDR2 comprises the amino acid sequence of SEQ ID NO: 192, the heavy chain CDR3 comprises the amino acid sequence of SEQ ID NO: 195, the light chain CDR1 comprises the amino acid sequence of SEQ ID NO: 201, the light chain CDR2 comprises the amino acid sequence of SEQ ID NO: 205, and the light chain CDR3 comprises the amino acid sequence of SEQ ID NO: 213.

7. The human antibody molecule of claim 5, wherein the heavy chain variable region comprises the amino acid sequence of SEQ ID NO: 108 and the light chain variable region comprises the amino acid sequence of SEQ ID NO: 110.

8. The human antibody molecule of claim 1, wherein the antibody comprises a human IgG1 heavy chain constant region.

9. The human antibody molecule of claim 8, wherein the human IgG1 heavy chain constant region comprises the amino acid sequence of SEQ ID NO: 122 or 124.

10. The human antibody molecule of claim 1, wherein the antibody comprises a human IgG2 heavy chain constant region.

11. The human antibody molecule of claim 10, wherein the human IgG2 heavy chain constant region comprises the amino acid sequence of SEQ ID NO: 120.

12. The human antibody molecule of claim 1, wherein the antibody comprises a human IgG4 heavy chain constant region.

13. The human antibody molecule of claim 1, wherein the antibody molecule is an Fab fragment, an F(ab)2 fragment, or a single chain antibody.

14. A pharmaceutical composition comprising the human antibody molecule of claim 1.

15. A nucleic acid molecule encoding the human antibody molecule of claim 1.

16. A vector comprising the nucleic acid molecule of claim 15.

17. A cell transformed to express the human antibody molecule of claim 1.

18. A method of treating or preventing airway neutrophilia or acute lung inflammation in a subject, the method comprising:
administering to the subject a therapeutically effective amount of the human antibody molecule of claim 1 or the pharmaceutical composition of claim 14 to prevent or treat the airway neutrophilia or acute lung inflammation.

19. The method of claim 18, wherein the airway neutrophilia or acute lung inflammation or both are chronic obstructive pulmonary disease, severe neutrophilic asthma, or both.

20. A method of blocking neutrophil chemotaxis comprising exposing neutrophils to the human antibody of claim 1.

21. The method of claim 20, wherein the chemotaxis is migration of neutrophils into the lung.

22. A method of blocking CXCR2 signaling in response to CXCR1 and/or CXCR5, in a cell expressing CXCR2 signaling, comprising exposing cells to the human antibody of claim 1.
